(19) United States
(12) Patent Application Publication
(10) Pub. No.: US 2011/0111014 A1
(43) Pub. Date: May 12, 2011

(54) METHODS AND COMPOSITIONS FOR TREATMENT OF NEUROLOGICAL DISORDERS

(75) Inventor: William J. Langston, Los Altos Hills, CA (US)

(73) Assignee: Parkinson's Institute, Sunnyvale, CA (US)

(21) Appl. No.: 12/666,626

(22) PCT Filed: Jun. 26, 2008
(86) PCT No.: PCT/US08/68442

§ 371 Date: Jan. 14, 2011

Related U.S. Application Data
(60) Provisional application No. 60/946,374, filed on Jun. 26, 2007.
(60) Provisional application No. 60/951,096, filed on Jul. 20, 2007.
(60) Provisional application No. 61/032,334, filed on Feb. 28, 2008.

(51) Int. Cl. A61K 9/127; A61K 31/485; A61K 31/352; A61K 31/195; A61K 31/12; A61P 25/00; A61P 25/28; A61P 25/16

(52) U.S. Cl. 424/450; 514/284; 514/456; 514/567; 514/685

(57) ABSTRACT

Methods and compositions are provided for preventing and treating neurological disorders, in particular Parkinson's disease.

### CROSS-REFERENCE

This application claims the benefit of U.S. Provisional Application No. 60/946,374, filed Jun. 26, 2007; U.S. Provisional Application No. 60/951,096, filed Jul. 20, 2007, and U.S. Provisional Application No. 61/032,334, filed Feb. 28, 2008, which applications are incorporated herein by reference in their entirety.

### GOVERNMENT INTERESTS

Certain embodiments of the present invention were made by the National Institutes of Health (NIH) under research grant number R01 NS048584, who may have certain rights thereto.

### BACKGROUND OF THE INVENTION

Neurological and neurodegenerative diseases are progressive degenerative disorders of the central nervous system characterized by a loss of neurons in particular regions of the brain. Parkinson's disease and Alzheimer's disease are two examples of such diseases where there is a loss of neuronal function to the dompaminergic neurons of the substantia nigra and the cholinergic neurons of the basal forebrain, respectively. In Parkinson's disease dopaminergic neurons, when functional, synthesize and release dopamine, a neurotransmitter used in chemical communication with other cells. Symptoms of Parkinson's disease, including rigidity, resting tremor (shaking), poverty of movement (akinesia), slowness of movement (bradykinesia), and changes in gait and posture, can be severely debilitating, causing a profound change in the quality of life for the spouse or caregiver as well as the patient. There is a need for therapies to inhibit, reverse, or prevent manifestation of nuerological disorders such as Parkinson's disease.

### SUMMARY OF THE INVENTION

In one embodiment a prophylactic method for a neurological disorder is provided, and said method comprises administering a compound selected from apomorphine, pyrogallol, 1,4-naphthoquinone, cisplatin, isoproterenol, pyrogallin, cianidanol, sulfasalazine, quinalizarin, benserazide, hexachlorophene, pyrvinium pamoate, dobutamine, methyl-dopa, curcumin, berberine chloride, daidzein, merbromin, norepinephrine, dopamine hydrochloride, carbidopa, ethylnorepinephrine hydrochloride, tannic acid, elaidyphosphocholine, hydroquinone, chlorophyllide Cu complex Na salt, methyldopa, isoproterenol hydrochloride, benserazide hydrochloride, dopamine, dobutamine hydrochloride, thyroid hormone, purpurin, sodium beta-nicotinamide adenine dinucleotide phosphate, lansoprazole, dyclonine hydrochloride, pramoxine hydrochloride, azobenzene, cefamandole sodium, cephaloridine, myricetin, 6,2′,3′-trihydroxyflavone, 5,7,3′,4′,5′-pentahydroxyflavone, 7,3′,4′,5′-tetrahydroxyflavone, (5,6,7,4′-tetrahydroxyflavone), baicalein, eriodictyol, 7,3′,4′-trihydroxyisoflavone, epigallocatechin gallate, quercetin, gossypetin (3,5,7,8,3′,4′-hexahydroxyflavone), 2′,3′-dihydroxyflavone, 3′,4′-dihydroxyflavone, 5,6-dihydroxy-7-methoxyflavone, baicalein-7-methyl ether, 1-dopa, DOPAC, homogentisic acid, 6-hydroxydopamine, epinephrine, 3,4-dihydroxycinnamic acid, 2,3-dihydroxynaphthalene, 3,4-dihydroxybenzoic acid, 3,4,5-trihydroxybenzoic acid, 1,2,3-trihydroxybenzoic acid, gallate (gallic acid), benzoquinone, catechol, rifampicin, rosmarinic acid, baicalin, tanshinones I and II, emodin, procyanidin B4, resveratrol, rutin, fisetin, luteolin, fustin, epicatechin gallate, catechin, alizarin, tannic acid, eriodyctol, carboplatin, purpurogallin-4-carboxylic acid, koparin, 2,3,4-trihydroxy-4′-ethexybenzophenone, baeomycesic acid, hamtoxylin, iriginol hexaaceatate, 4-acetoxyphenol, theaflavin monogallate, theaflavin digallate, stictic acid, purpurogallin, 2,5-dihydroxy-3,4-dimethoxy-4′-ethoxybenzophenone, promethazine hydrochloride, oxidopamine hydrochloride, pyrantel pamoate, elaidylphosphocholine, amphotericin B, gallic acid, fumarprotocetraric acid, theaflavin, haematoxylin pentaacetate, 4-methoxydalbergione, epigallocatechin-3-monogallate, rolitetracycline, 7,3′-dimethoxyflavone, liquiritigenin dimethyl ether, catechin pentaacetate, apigenin, 3,4-dedesmethyl-5-deshydroxy-3′-ethoxyscleroin or derivatives thereof to a subject at risk of developing the disorder. In one aspect the disorder is selected from a group consisting of Parkinson's disease, Alzheimer's disease, Huntington's disease, multiple system atrophy or Lewy body disease. In another aspect the disorder is Parkinson's disease.

In another embodiment, the method of treating a subject at risk of developing a neurological disorder is provided and said method comprises administering one or more compounds selected from apomorphine, pyrogallol, 1,4-naphthoquinone, cisplatin, isoproterenol, pyrogallin, cianidanol, sulfasalazine, quinalizarin, benserazide, hexachlorophene, pyrvinium pamoate, dobutamine, methyl-dopa, curcumin, berberine chloride, daidzein, merbromin, norepinephrine, dopamine hydrochloride, carbidopa, ethylnorepinephrine hydrochloride, tannic acid, elaidyphosphocholine, hydroquinone, chlorophyllide Cu complex Na salt, methyldopa, isoproterenol hydrochloride, benserazide hydrochloride, dopamine, dobutamine hydrochloride, thyroid hormone, purpurin, sodium beta-nicotinamide adenine dinucleotide phosphate, lansoprazole, dyclonine hydrochloride, pramoxine hydrochloride, azobenzene, cefamandole sodium, cephaloridine, myricetin, 6,2′,3′-trihydroxyflavone, 5,7,3′,4′,5′-pentahydroxyflavone, 7,3′,4′,5′-tetrahydroxyflavone, (5,6,7,4′-tetrahydroxyflavone), baicalein, eriodictyol, 7,3′,4′-trihydroxyisoflavone, epigallocatechin gallate, quercetin, gossypetin (3,5,7,8,3′,4′-hexahydroxyflavone), 2′,3′-dihydroxyflavone, 3′, 4′-dihydroxyflavone, 5,6-dihydroxy-7-methoxyflavone, baicalein-7-methyl ether, 1-dopa, DOPAC, homogentisic acid, 6-hydroxydopamine, epinephrine, 3,4-dihydroxycinnamic acid, 2,3-dihydroxynaphthalene, 3,4-dihydroxybenzoic acid, 3,4,5-trihydroxybenzoic acid, 1,2,3-trihydroxybenzoic acid, gallate (gallic acid), benzoquinone, catechol, rifampicin, rosmarinic acid, baicalin, tanshinones I and II, emodin, procyanidin B4, resveratrol, rutin, fisetin, luteolin, fustin, epicatechin gallate, catechin, alizarin, tannic acid, eriodyctol, carboplatin, purpurogallin-4-carboxylic acid, koparin, 2,3,4-trihydroxy-4′-ethexybenzophenone, baeomycesic acid, hamtoxylin, iriginol hexaaceatate, 4-acetoxyphenol, theaflavin monogallate, theaflavin digallate, stictic acid, purpurogallin, 2,5-dihydroxy-3,4-dimethoxy-4′-ethoxybenzophenone, promethazine hydrochloride, oxidopamine hydrochloride, pyrantel pamoate, elaidylphosphocholine, amphotericin B, gallic acid, fumarprotocetraric acid, theaflavin, haematoxylin pentaacetate, 4-methoxydalbergione, epigallocatechin-3-monogallate, rolitetracycline, 7,3′-dimethoxyflavone, liquiritigenin dimethyl ether, catechin pentaacetate, apigenin, 3,4-dedesmethyl-5-deshydroxy-3′-ethoxyscleroin or derivatives thereof to a subject, wherein the subject does not show any primary symptoms associated with said disorder and wherein the administering delays or prevents onset of primary symptoms of the disorder. In one aspect, the disorder is selected from a group consisting of Parkinson's disease, Alzheimer's disease, Huntington's disease, multiple system atrophy or Lewy body disease. In another aspect, the disorder is Parkinson's disease. In some aspects the subject displays at least two secondary symptoms associated with said disorder, or at least one secondary symptom and carries at least one genetic marker associated with said disorder, or displays at least one secondary symptom and has at least one biomarker associated with said disorder, or displays no secondary symptoms prior to administering.

In another embodiment a method of preventing or reversing α-synuclein fibrillation in a subject with or at risk of developing a neurological disorder is provided and the method comprises administering an effective amount of a compound that prevents or reverses α-synuclein fibrillation, wherein the compound is selected from apomorphine, pyrogallol, 1,4-naphthoquinone, cisplatin, isoproterenol, pyrogallin, cianidanol, sulfasalazine, quinalizarin, benserazide, hexachlorophene, pyrvinium pamoate, dobutamine, methyl-dopa, curcumin, berberine chloride, daidzein, merbromin, norepinephrine, dopamine hydrochloride, carbidopa, ethylnorepinephrine hydrochloride, tannic acid, elaidyphosphocholine, hydroquinone, chlorophyllide Cu complex Na salt, methyldopa, isoproterenol hydrochloride, benserazide hydrochloride, dopamine, dobutamine hydrochloride, thyroid hormone, purpurin, sodium beta-nicotinamide adenine dinucleotide phosphate, lansoprazole, dyclonine hydrochloride, pramoxine hydrochloride, azobenzene, cefamandole sodium, cephaloridine, myricetin, 6,2′,3′-trihydroxyflavone, 5,7,3′4′,5′-pentahydroxyflavone, 7,3′,4′,5′-tetrahydroxyflavone, (5,6,7,4′-tetrahydroxyflavone), baicalein, eriodictyol, 7,3′,4′-trihydroxyisoflavone, epigallocatechin gallate, quercetin, gossypetin (3,5,7,8,3′,4′-hexahydroxyflavone), 2′,3′-dihydroxyflavone, 3′,4′-dihydroxyflavone, 5,6-dihydroxy-7-methoxyflavone, baicalein-7-methyl ether, 1-dopa, DOPAC, homogentisic acid, 6-hydroxydopamine, epinephrine, 3,4-dihydroxycinnamic acid, 2,3-dihydroxynaphthalene, 3,4-dihydroxybenzoic acid, 3,4,5-trihydroxybenzoic acid, 1,2,3-trihydroxybenzoic acid, gallate (gallic acid), benzoquinone, catechol, rifampicin, rosmarinic acid, baicalin, tanshinones I and II, emodin, procyanidin B4, resveratrol, rutin, fisetin, luteolin, fustin, epicatechin gallate, catechin, alizarin, tannic acid, eriodyctol, carboplatin, purpurogallin-4-carboxylic acid, koparin, 2,3,4-trihydroxy-4′-ethexybenzophenone, baeomycesic acid, hamtoxylin, iriginol hexaaceatate, 4-acetoxyphenol, theaflavin monogallate, theaflavin digallate, stictic acid, purpurogallin, 2,5-dihydroxy-3,4-dimethoxy-4′-ethoxybenzophenone, promethazine hydrochloride, oxidopamine hydrochloride, pyrantel pamoate, elaidylphosphocholine, amphotericin B, gallic acid, fumarprotocetraric acid, theaflavin, haematoxylin pentaacetate, 4-methoxydalbergione, epigallocatechin-3-monogallate, rolitetracycline, 7,3′-dimethoxyflavone, liquiritigenin dimethyl ether, catechin pentaacetate, apigenin, 3,4-dedesmethyl-5-deshydroxy-3′-ethoxyscleroin or derivatives thereof. In one aspect the disorder is selected from a group consisting of Parkinson's disease, Alzheimer's disease, Huntington's disease, multiple system atrophy or Lewy body disease. In another aspect, the disorder is Parkinson's disease.

In some embodiments of the invention, the subject is at risk of developing the neurological disorder and risk is determined by genetic testing of a sample from the subject, or by testing for biomarkers from a sample from the subject, or by screening for secondary symptoms. In one aspect the neurological disease is Parkinson's disease and the secondary symptom is selected from the group consisting of rapid eye movement sleep behavioral disorder, olfactory dysfunction, cardiac sympathetic denervation, constipation, depression, anxiety and dementia.

In other embodiments of the invention, a subject is treated with apomorphine, epigallocatechin gallate, baicalein, quercetin, or curcumin. In certain aspects the compound is administered via an oral route, continuous duodenal infusion, rectal, intranasal, sublingual and subcutaneous, skin patches, prodrug-dispensing liposomes, pulmonary delivery or a combination. In other aspects the subject is administered an additional therapeutic agent to achieve a therapeutic effect in combination with the compound. In one aspect the additional therapeutic agent is selected from a group consisting of an anti-emetic, 1-dihydroxyphenylalanine, aromatic acid decarboxylase inhibitor, catechol-O-methyltransferase inhibitor, monoamine oxidase inhibitor, tocopherol, a different dopamine agonist, otigotine, lisuride, nicotinic receptor agonist, amantadine, carbidopa, entacapone, levodopa, bromocriptine, pergolide, pramixpexole, cabergoline, ropinorole, or a combination of two or more thereof. In certain aspects the therapeutic effect comprises reducing said risk of developing said disorder. In some aspects the delivering is before, concurrent or after administering of compound. In one aspect the additional agent is levodopa, a nicotine receptor modulator, or a monoamine oxidase inhibor.

In another embodiment a method of treating Parkinson's disease in a subject is provided and comprises administering to a subject an effective amount of any compound of pyrogallol, 1,4-naphthoquinone, cisplatin, isoproterenol, pyrogallin, cianidanol, sulfasalazine, quinalizarin, benserazide, hexachlorophene, pyrvinium pamoate, dobutamine, methyl-dopa, curcumin, berberine chloride, daidzein, merbromin, dopamine hydrochloride, carbidopa, ethylnorepinephrine hydrochloride, tannic acid, elaidyphosphocholine, hydroquinone, chlorophyllide Cu complex Na salt, methyldopa, isoproterenol hydrochloride, hydrochloride, dopamine, dobutamine hydrochloride, thyroid hormone, purpurin, sodium beta-nicotinamide adenine dinucleotide phosphate, lansoprazole, dyclonine hydrochloride, pramoxine hydrochloride, azobenzene, cefamandole sodium, cephaloridine, myricetin, 6,2′,3′-trihydroxyflavone, 5,7,3′,4′,5′-pentahydroxyflavone, 7,3′,4′,5′-tetrahydroxyflavone, (5,6,7,4′-tetrahydroxyflavone), baicalein, eriodictyol, 7,3′,4′-trihydroxyisoflavone, epigallocatechin gallate, quercetin, gossypetin (3,5,7,8,3′,4′-hexahydroxyflavone), 2′,3′-dihydroxyflavone, 3′,4′-dihydroxyflavone, 5,6-dihydroxy-7-methoxyflavone, baicalein-7-methyl ether, DOPAC, homogentisic acid, 6-hydroxydopamine, epinephrine, 3,4-dihydroxycinnamic acid, 2,3-dihydroxynaphthalene, 3,4-dihydroxybenzoic acid, 3,4,5-trihydroxybenzoic acid, 1,2,3-trihydroxybenzoic acid, gallate (gallic acid), benzoquinone, catechol, rifampicin, rosmarinic acid, baicalin, tanshinones I and II, emodin, procyanidin B4, resveratrol, rutin, fisetin, luteolin, fustin, epicatechin gallate, catechin, alizarin, tannic acid, eriodyctol, carboplatin, purpurogallin-4-carboxylic acid, koparin, 2,3,4-trihydroxy-4′-ethexybenzophenone, baeomycesic acid, hamtoxylin, iriginol hexaaceatate, 4-acetoxyphenol, theaflavin monogallate, theaflavin digallate, stictic acid, purpurogallin, 2,5-dihydroxy-3,4-dimethoxy-4′-ethoxybenzophenone, promethazine hydrochloride, oxidopamine hydrochloride, pyrantel pamoate, elaidylphosphocholine, amphotericin B, gallic acid, fumarprotocetraric acid, theaflavin, haematoxylin pentaacetate, 4-methoxydalbergione, epigallocatechin-3-monogallate, rolitetracycline, 7,3′-dimethoxyflavone, liquiritigenin dimethyl ether, catechin pentaacetate, apigenin, 3,4-dedesmethyl-5-deshydroxy-3′-ethoxyscleroin or derivatives thereof. In one aspect the compound is epigallocatechin gallate, baicalein, quercetin, curcumin, or derivatives thereof. In certain aspects administering is via an oral route, continuous duodenal infusion, rectal, intranasal, sublingual and subcutaneous, skin patches, prodrug-dispensing liposomes, pulmonary delivery or a combination thereof. In another aspect an additional therapeutic agent is delivered to achieve a therapeutic effect in combination with the compound. In some aspects, the additional therapeutic agent is selected from a group consisting of an anti-emetic, 1-dihydroxyphenylalanine, aromatic acid decarboxylase inhibitor, catechol-O-methyltransferase inhibitor, monoamine oxidase inhibitor, tocopherol, a different dopamine agonist, otigotine, lisuride, nicotinic receptor agonist, amantadine, carbidopa, entacapone, levodopa, bromocriptine, pergolide, pramixpexole, cabergoline, ropinorole, or a combination of two or more thereof.

In another embodiment a method of treating a subject at risk of developing a neurological disorder is provided and the method comprises administering either a dopamine agonist, a monoamine oxidase inhibitor, a nutraceutical, or an inhibitor of α-synuclein fibrillation to a subject who does not show any primary symptoms associated with the disorder; and wherein said administering delays or prevents onset of primary symptoms of the disorder. In one aspect the neurological disorder is Parkinson's disease. In certain aspects, the risk of developing Parkinson's disease is determined by one or more of the following methods: screening for a genetic trait, screening for secondary symptoms, or screening for biomarkers. In certain aspects the genetic trait can be a mutation and the mutation can be a α-synuclein substitution, deletion, insertion, triplication and duplication. In another aspect the genetic trait is a polymorphism. In certain other aspects the polymorphism is selected from the group consisting of SNP, STR and VNTR. In one aspect the administering of the compound is via an oral route, continuous duodenal infusion, rectal, intranasal, sublingual and subcutaneous, skin patches, prodrug-dispensing liposomes, pulmonary delivery or a combination thereof. In certain aspects an additional therapeutic agent to achieve a therapeutic effect in combination with said compound can be delivered. In some aspects the additional therapeutic agent is selected from a group consisting of an anti-emetic, 1-dihydroxyphenylalanine, aromatic acid decarboxylase inhibitor, catechol-O-methyltransferase inhibitor, monoamine oxidase-B inhibitor, a different dopamine agonist, otigotine, lisuride, nicotinic receptor agonist, amantadine, carbidopa, entacapone, levodopa, a tocopherol, bromocriptine, pergolide, pramixpexole, cabergoline, ropinorole, or a combination of two or more thereof. In one aspect the therapeutic effect comprises enhancing said delayed progression and/or decreased side effects associated with said administering of compound. In certain aspects the additional therapeutic agent is delivered before, concurrent or after said administering of compound. In one aspect the additional therapeutic agent is levodopa, a nicotinic receptor modulator, or a monoamine oxidase inhibitor.

In another embodiment of the invention a method of treating a subject at risk of or suffering from a neurological disorder is provided and comprises administering xxx wherein said administering decreases, delays, prevents, or reverses primary symptoms of the disorder. In one aspect the compound is epigallocatechin gallate, baicalein, quercetin, or curcumin, a metabolite, analog or derivative thereof. In another aspect administering is via an oral route, continuous duodenal infusion, rectal, intranasal, sublingual and subcutaneous, skin patches, prodrug-dispensing liposomes, pulmonary delivery or a combination thereof. In another aspect, an additional therapeutic agent is delivered to achieve a therapeutic effect in combination with the compound. In certain aspects the additional therapeutic agent is selected from a group consisting of an anti-emetic, 1-dihydroxyphenylalanine, aromatic acid decarboxylase inhibitor, catechol-O-methyltransferase inhibitor, monoamine oxidase inhibitor, tocopherol, a different dopamine agonist, otigotine, lisuride, nicotinic receptor agonist, amantadine, carbidopa, entacapone, levodopa, bromocriptine, pergolide, pramixpexole, cabergoline, ropinorole, or a combination of two or more thereof.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows results from experiments with isoproterenol.

FIG. 2 shows results from experiments with epicatechin.

FIG. 3 shows results from experiments with pyrogallin.

FIG. 4 shows results from experiments with cisplatin.

FIG. 5 shows results from experiments with quinalizarin.

FIG. 6 shows results from experiments with methyldopa.

FIG. 7 shows results from experiments with carbidopa.

FIG. 8 shows results from experiments with dobutamine.

FIG. 9 shows results from experiments with pyrvinium pamoate.

FIG. 10 shows results from experiments with chlorophyllide Cu complex Na salt.

FIG. 11 shows results from experiments with elaidylphosphocholine.

FIG. 12 shows results from experiments with apomorphine.

FIG. 13 shows results from experiments with baicalein.

FIG. 14 shows results from experiments with oxidopamine hydrochloride.

FIG. 15 shows results from experiments with benserazide hydrochloride.

FIG. 16 shows results from experiments with promethazine hydrochloride

FIG. 17 shows results from experiments with pyrantel pamoate.

FIG. 18 shows results from experiments with tannic acid.

FIG. 19 shows results from experiments with elaidylphosphocholine.

FIG. 20 shows results from experiments with amphotericin B.

FIG. 21 shows results from experiments with eriodyctol.

FIG. 22 shows results from experiments with gallic acid.

FIG. 23 shows results from experiments with purpurogallin-4-carboxylic Acid.

FIG. 24 shows results from experiments with koparin.

FIG. 25 shows results from experiments with 2,3,4-trihydroxy-4′-ethexybenzophenone.

FIG. 26 shows results from experiments with baeomycesic Acid.

FIG. 27 shows results from experiments with iriginol hexaaceatate.

FIG. 28 shows results from experiments with hamtoxylin.

FIG. 29 shows results from experiments with 4-acetoxyphenol.

FIG. 30 shows results from experiments with theaflavin.

FIG. 31 shows results from experiments with haematoxylin pentaacetate.

FIG. 32 shows results from experiments with 4-methoxydalbergione.

FIG. 33 shows results from experiments with theaflavin monogallate.

FIG. 34 shows results from experiments with theaflavin digallate.

FIG. 35 shows results from experiments with epigallocatechin-3-monogallate.

FIG. 36 shows results from experiments with stictic acid

FIG. 37 shows results from experiments with isoproterenol hydrochloride

FIG. 38 shows results from experiments with rolitetracycline.

FIG. 39 shows results from experiments with 7,3′-dimethoxyflavone.

FIG. 40 shows results from experiments with liquiritigenin dimethyl ether.

FIG. 41 shows results from experiments with catechin pentaacetate

FIG. 42 shows results from experiments with apigenin.

FIG. 43 shows results from experiments with purpurogallin.

FIG. 44 shows results from experiments with 2,5-dihydroxy-3,4-dimethoxy-4′-ethoxybenzophenone.

FIG. 45 shows results from experiments with 3,4-dedesmethyl-5-deshydroxy-3′-ethoxyscleroin.

FIG. 46 shows results from experiments with atomic force microscopy for detection of fibrillation.

### DETAILED DESCRIPTION OF THE INVENTION

Reference will now be made in detail to several embodiments of the invention. Examples of the certain embodiments are illustrated in the following Examples section.

Unless defined otherwise, all technical and scientific terms used herein have the same meaning as is commonly understood by one of skill in the art to which this invention belongs. All patents and publications referred to herein are incorporated by reference.

The invention provides compositions and methods utilizing such compositions. In some embodiments the invention provides for compositions and methods for utilizing compounds to treat subject at risk of a neurological disorder or reverse the progression of a subject suffering from a neurological disorder. In some embodiments, the invention provides for compositions and methods of utilizing compounds that inhibit, decrease, reverse, or prevent α-synuclein fibrillation and/or aggregation. In some embodiments the compound is selected from apomorphine, apomorphine derivatives, pyrogallol, 1,4-naphthoquinone, cisplatin, isoproterenol, pyrogallin, cianidanol, sulfasalazine, quinalizarin, benserazide, hexachlorophene, pyrvinium pamoate, dobutamine, methyl-dopa, curcumin, berberine chloride, daidzein, merbromin, norepinephrine, dopamine hydrochloride, carbidopa, ethylnorepinephrine hydrochloride, tannic acid, elaidyphosphocholine, hydroquinone, chlorophyllide Cu complex Na salt, methyldopa, isoproterenol hydrochloride, benserazide hydrochloride, dopamine, dobutamine hydrochloride, thyroid hormone, purpurin, sodium beta-nicotinamide adenine dinucleotide phosphate, lansoprazole, dyclonine hydrochloride, pramoxine hydrochloride, azobenzene, cefamandole sodium, cephaloridine, myricetin, 6,2′,3′-trihydroxyflavone, 5,7,3′,4′,5′-pentahydroxyflavone, 7,3′,4′,5′-tetrahydroxyflavone, (5,6,7,4′-tetrahydroxyflavone), baicalein, eriodictyol, 7,3′,4′-trihydroxyisoflavone, epigallocatechin gallate, quercetin, gossypetin (3,5,7,8,3′,4′-hexahydroxyflavone), 2′,3′-dihydroxyflavone, 3′,4′-dihydroxyflavone, 5,6-dihydroxy-7-methoxyflavone, baicalein-7-methyl ether, 1-dopa, DOPAC, homogentisic acid, 6-hydroxydopamine, epinephrine, 3,4-dihydroxycinnamic acid, 2,3-dihydroxynaphthalene, 3,4-dihydroxybenzoic acid, 3,4,5-trihydroxybenzoic acid, 1,2,3-trihydroxybenzoic acid, gallate (gallic acid), benzoquinone, catechol, rifampicin, rosmarinic acid, baicalin, tanshinones I and II, emodin, procyanidin B4, resveratrol, rutin, fisetin, luteolin, fustin, epicatechin gallate, catechin, alizarin, tannic acid, eriodyctol, carboplatin, purpurogallin-4-carboxylic acid, koparin, 2,3,4-trihydroxy-4′-ethexybenzophenone, baeomycesic acid, hamtoxylin, iriginol hexaaceatate, 4-acetoxyphenol, theaflavin monogallate, theaflavin digallate, stictic acid, purpurogallin, 2,5-dihydroxy-3,4-dimethoxy-4′-ethoxybenzophenone, promethazine hydrochloride, oxidopamine hydrochloride, pyrantel pamoate, elaidylphosphocholine, amphotericin B, gallic acid, fumarprotocetraric acid, theaflavin, haematoxylin pentaacetate, 4-methoxydalbergione, epigallocatechin-3-monogallate, rolitetracycline, 7,3′-dimethoxyflavone, liquiritigenin dimethyl ether, catechin pentaacetate, apigenin, 3,4-dedesmethyl-5-deshydroxy-3′-ethoxyscleroin, derivatives thereof, or a combination thereof. In some embodiments, the compounds and compositions described herein are utilized to prevent or slow down the progression of Parkinson's disease.

### Dopamine Agonists and Apomorphine

A dopamine agonist is a compound that activates dopamine receptors, mimicking the effect of the neurotransmitter dopamine. Examples of dopamine agonists include but are not limited to: bromocriptine, pergolide, pramipexole, ropinirole , cabergoline, apomorphine, and lisuride are moderately effective. Apomorphine has recently been employed in conjunction with other medications in the treatment of Parkinson's disease patients who have become resistant to chronic levodopa therapy or who have developed abnormal movements as a consequence of chronic levodopa therapy. Apomorphine hydrochloride is chemically designated as 6αβ-Aporphine-10,11-diol hydrochloride hemihydrate with a molecular formula of C₁₇H₁₇NO₂HCl ½H₂O. Its structural formula is:

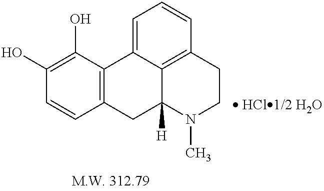

When a subject is administered apomorphine, metabolites are generated in vivo such as esters, ethers, amides, mixed anhydrides, hemiacetals, glucuronates, sulfates and phosphonates. Some specific examples include morapomorphine, which is N-demethylated apomorphine or O- or N-substituted compounds such as sulfate or glucuronide derivatives of apomorphine and norapomorphine, among others. Such derivatives are also available synthetically. The invention provides compositions and method utilizing such compositions. In some embodiments, the invention provides for compositions and methods of utilizing apomorphine or apomorphine derivatives. Examples of apomorphine derivatives include, but are not limited to, apomorphine N-glucuronide, apomorphine O-glucuronide, apomorphine O-sulfate, apomorphine N-sulfate, norapomorphine, norapomorphine O-glucuronide, norapomorphine N-glucuronide, norapomorphine O-sulfate, norapomorphine N-sulfate and combinations thereof. If produced in vivo as a result of treatment with apomorphine, these derivatives may be secreted in bile and may be hydrolyzed in the intestinal lumen, and thereafter circulate enterohepatically. In some embodiments, the subject is treated with the derivatives directly.

Monoamine Oxidase and Monoamine Oxidase Inhibitors

There are two isoforms of monoamine oxidase (MAO), MAO-A and MAO-B. MAO-A preferentially deaminates serotonin, melatonin, epinephrine and norepinephrine. MAO-B preferentially deaminates phenylethylamine and trace amines. Dopamine is equally deaminated by both types.

Monoamine oxidase inhibitors (MAOIs) act by inhibiting the activity of monoamine oxidase preventing the breakdown of monoamine neurotransmitters, which increases their availability. MAOIs can irreversibly or transiently inhibit MAO. In addition to reversibility, MAOIs differ by their selectivity of the MAO receptor. Some MAOIs inhibit both MAO-A and MAO-B equally, other MAOIs have been developed that target one over the other. Examples of MAOIs include but are not limited to: 2-(3,4-dimethoxyphenyl)-3-fluoroallylamine, 7,8-dichloro-1,2,3,4-tetrahydroisoquinoline, amezinium, amiflamine, brofaromine, chlorphenamidine, cimoxatone, clorgyline, cuprizone, desmethylselegiline, daprenyl, furazolidone, harmaline, harmine, iproniazid, isocarboxazid, lazabemide, milacemide, moclobemide, mofegiline, monocrotophos, nialamide, pargyline, phenelzine, pirlindole, rasagiline, selegiline, toloxatone, tranylcypromine, or tribulin.

MAOIs prolong the effects of dopamine in the brain by preventing its breakdown. They also can prevent the removal of dopamine between nerve endings and enhance release of dopamine from nerve cells. In certain embodiments inhibition of MAO can be effective in slowing the progression to Parkinson's disease by preventing the onset of motor symptoms of Parkinson's disase, and/or preventing death of dopaminergic neurons when administered early enough. In certain embodiments a MAOI can be administered in combination with another compound to further prevent the onset of motor symptoms associated with Parkinson's disease. In one aspect prophylactic treatment for a neurological disease comprises administering one or more MAOIs prior to progression of motor symptoms by the subject.

α-Synuclein

α-synuclein is one of hundreds of proteins in mammalian cells, with expression in the brain and throughout the body. α-synuclein when misfolded creates fibrils. Fibrils can aggregate and are either too dense or too large to be flushed from the cell by lysosomes and/or proteasomes. As the α-synuclein protein aggregates, it can collect additional proteins and eventually turn into a “Lewy body” or a “Lewy neurite.” α-synuclein-related pathology is involved in the etiology of a variety of neurological disorders, including Parkinson's Disease, Parkinson's Disease with accompanying dementia, Lewy body dementia, Lewy body variant of Alzheimer's disease, Huntington's disease, Alzheimer's disease with Parkinsonism, and multiple system atrophy. Abnormal protein aggregates are a common pathological feature of many neurodegenerative diseases, as represented in Table 1.

TABLE 1

| | | Protein | Locus of |
|---|---|---|---|
| | Pathological | Component | mutation in |
| Disease | Feature | of Inclusion | familial case |
| Parkinson's | Lewy Body | α-synuclein | α-synuclein |
| Disease | Lewy Neurite | | |
| Alzheimer's | Senile Plaque | amyloid | amyloid |
| Disease | Neurofibrillary | β-peptide | precursor |
| | Tangle | Tau | protein |
| | | | Presenilin 1, 2 |
| Frontotemporal | Neurofibrillary | Tau | Tau |
| Dementia with | Tangle | | |
| Parkinsonism | | | |
| Chromosome-17 | | | |
| Huntington's | Nuclear and | Huntingtin | Huntingtin |
| Disease | Cytoplasmic | | |
| | Inclusion | | |
| Spinocerebellar | Nuclear and | Ataxins | Ataxins |
| Ataxias | Cytoplasmic | | |
| | Inclusion | | |
| Amyotrophic Lateral | Cytoplasmic | SOD1 | SOD1 |
| Sclerosis | and Axonal | Neurofilaments | NF-H |
| | Inclusion | | |
| Creutzfeldt-Jakob | Amyloid | Prion | Prion |
| Disease | Deposit | | |

Without being limited to any theory, it is thought that, when α-synuclein is for example overexpressed, temporally aberrantly expressed, spatially aberrantly expressed, mistargeted, genetically mutated, environmentally mutated, damaged by system failure such as in old age or injury, the protein takes on an aberrant shape or conformation that can be impressed upon other synuclein molecules. These molecules then bind to one another and the resulting protein aggregates accumulate and deposit inside the neuron, where they exert oxidative and other types of damage as they increase in size. This process prevents the neuron from performing its necessary role in brain function and can eventually result in neuronal cell death.

Furthermore, the α-synuclein fragment is a constituent of Alzheimer's disease amyloid plaques, hence the alternative name of non-amyloid component (NAC). An additional pathophysiological involvement of α-synuclein relates to the previously unrecognized high incidence of Lewy body dementia. In the absence of neuropathological evidence, Lewy body dementia is frequently misdiagnosed as Alzheimer's disease.

Sporadic and familial Parkinson's disease exhibit similar clinical manifestations and neuropathological profiles (e.g., Lewy bodies and Lewy neurites in the substantia nigra and other brain regions). Aggregated α-synuclein is the principal component of Lewy bodies and Lewy neurites (Spillantini et al., 1998). In fact, Lewy bodies and Lewy neurites are requisite for the diagnosis of Parkinson's disease and Lewy body dementia. Therefore, α-synuclein fibrillation and/or aggregation is a target for treating associated neurological disorders.

In some aspects of the invention compositions and methods are provided for utilizing one or more compounds that are α-synuclein modulators which inhibit, decrease, reverse, or prevent α-synuclein fibrillation and/or aggregation. In other aspects of the invention compositions and methods are provided for utilizing one or more compounds which prevent the onset of the symptoms associated with a neurological disorder, inhibit the progression of a neurological disorder, or ameliorate symptoms of a neurological disorder. In various embodiments, such a compound or combination of compounds include but is not limited to apomorphine, apomorphine derivatives, pyrogallol, 1,4-naphthoquinone, cisplatin, isoproterenol, pyrogallin, cianidanol, sulfasalazine, quinalizarin, benserazide, hexachlorophene, pyrvinium pamoate, dobutamine, methyl-dopa, curcumin, berberine chloride, daidzein, merbromin, norepinephrine, dopamine hydrochloride, carbidopa, ethylnorepinephrine hydrochloride, tannic acid, elaidyphosphocholine, hydroquinone, chlorophyllide Cu complex Na salt, methyldopa, isoproterenol hydrochloride, benserazide hydrochloride, dopamine, dobutamine hydrochloride, thyroid hormone, purpurin, sodium beta-nicotinamide adenine dinucleotide phosphate, lansoprazole, dyclonine hydrochloride, pramoxine hydrochloride, azobenzene, cefamandole sodium, cephaloridine, myricetin, 6,2′,3′-trihydroxyflavone, 5,7,3′,4′,5′-pentahydroxyflavone, 7,3′,4′,5′-tetrahydroxyflavone, (5,6,7,4′-tetrahydroxyflavone), baicalein, eriodictyol, 7,3′,4′-trihydroxyisoflavone, epigallocatechin gallate, quercetin, gossypetin (3,5,7,8,3′,4′-hexahydroxyflavone), 2′,3-dihydroxyflavone, 3′,4′-dihydroxyflavone, 5,6-dihydroxy-7-methoxyflavone, baicalein-7-methyl ether, 1-dopa, DOPAC, homogentisic acid, 6-hydroxydopamine, epinephrine, 3,4-dihydroxycinnamic acid, 2,3-dihydroxynaphthalene, 3,4-dihydroxybenzoic acid, 3,4,5-trihydroxybenzoic acid, 1,2,3-trihydroxybenzoic acid, gallate (gallic acid), benzoquinone, catechol, rifampicin, rosmarinic acid, baicalin, tanshinones I and II, emodin, procyanidin B4, resveratrol, rutin, fisetin, luteolin, fustin, epicatechin gallate, catechin, alizarin, tannic acid, eriodyctol, carboplatin, purpurogallin-4-carboxylic acid, koparin, 2,3,4-trihydroxy-4′-ethexybenzophenone, baeomycesic acid, hamtoxylin, iriginol hexaaceatate, 4-acetoxyphenol, theaflavin monogallate, theaflavin digallate, stictic acid, purpurogallin, 2,5-dihydroxy-3,4-dimethoxy-4′-ethoxybenzophenone, promethazine hydrochloride, oxidopamine hydrochloride, pyrantel pamoate, elaidylphosphocholine, amphotericin B, gallic acid, fumarprotocetraric acid, theaflavin, haematoxylin pentaacetate, 4-methoxydalbergione, epigallocatechin-3-monogallate, rolitetracycline, 7,3′-dimethoxyflavone, liquiritigenin dimethyl ether, catechin pentaacetate, apigenin, 3,4-dedesmethyl-5-deshydroxy-3′-ethoxyscleroin, derivatives thereof, or a combination thereof. In some embodiments, the compositions of the invention are utilized to prevent or slow the onset or progression of Parkinson's disease.

### Nutraceuticals

In various embodiments, a compound that prevents the onset of the symptoms associated with a neurological disorder or inhibits and reverses the progression of a neurological disorders is provided. In some embodiments compounds are nutraceuticals. A nutraceutical refers to an extract of a food with potential medicinal effects, physiological benefits or can provide protection against a chronic disease. Nutraceuticals are consumed as part of a usual diet but are demonstrated to have physiological benefits and/or reduce the risk of chronic disease beyond basic nutritional functions. Examples of nutraceuticals are resveratrol from red grape products as an antioxidant, soluble dietary fiber products, such as psyllium seed husk for reducing hypercholesterolemia, broccoli (sulforaphane) as a cancer preventative, curcumin (turmeric) with a variety of functions in the brain and in cancerous cells and soy or clover (isoflavonoids) to improve arterial health. Other nutraceutical examples are flavonoids antioxidants, alpha-linolenic acid from flax seeds, beta-carotene from marigold petals, anthocyanins from berries, etc. Many botanical and herbal extracts are classified as nutraceuticals. Nutraceuticals can exhibit dopamine agonism, α-synuclein modulation, or MAO inhibition in addition to several other physiological functions and mechanisms. In some embodiments nutraceuticals can be administered to a subject at risk for developing a neurological disease or suffering from a neurological disease, alone or in combination with another drug, including but not limited to apomorphine and its derivatives, dopamine agonists, MAOIs, α-synuclein modulators or other nutraceuticals.

### Neurological Disorders

In various embodiments, a compound that prevents the onset of the symptoms associated with a neurological disorder, inhibits the progression of a neurological disorder, ameliorates symptoms of a neurological disorder, or inhibits, decreases, reverses, or prevents α-synuclein fibrillation and/or aggregation is administered to a subject at risk of developing or suffering from a neurological disorder alone or in combination with one, two or more active agents. It will be evident, that various risk factors known for a particular disorder can be utilized to screen a subject, in order to determine whether the subject is at risk of developing the particular disorder. Therefore, in various embodiments, such neurological disorders include, but are not limited to, ADHD, Alzheimer's disease (AD), amyotrophic lateral sclerosis (ALS; Lou Gehrig's disease), Bell's Palsy, Cerebral Palsy, chemotherapy-induced neuropathies (e.g., from vincristine, paclitaxel, bortezomib), chorea-acanthocytosis, Creutzfeldt-Jakob Disease (CJD), Dementia, diabetes-induced neuropathies, diffuse Lewy body disease, Epilepsy, Essential Tremor, Friedreich's ataxia, Guillain-Barre Syndrome, Hemifacial Spasm, Huntington's disease (HD), Movement Disorders, Multiple Sclerosis, Multisystem Atrophy (MSA), Nervous System Tumors, Neurofibromatosis, Neuropathy, ocular diseases (ocular neuritis), Parkinson's disease (PD), Periodic Limb Movement Disorder, primary lateral sclerosis, Seizure Disorders, Tourette's Syndrome or Traumatic Brain Injury. In some embodiments, these diseases involve α-synuclein and a compound that inhibits, decreases, reverses, or prevents α-synuclein fibrillation and/or aggregation alone or in combination with one, two or more active agents is administered to a subject who is at risk of developing or is suffering from Parkinson's Disease, Alzheimer's Disease or a Neuropathy. In other embodiments these diseases involve MAO and a MAOI alone or in combination with one, two or more active agents is administered to a subject who is at risk of developing or is suffering from Parkinson's Disease Furthermore, in some embodiments, such administration is to a subject that is suffering from the early stages of Parkinson's Disease, Alzheimer's Disease or a Neuropathy. In addition, where two or more active agents are administered, in various embodiments, administration is through the same or different routes of administration. Furthermore, in some embodiments, administration of two or more compounds occurs at temporally distinct timeframes and dosages. For example, a first compound may be administered before, during or after administration of a second compound.

### Parkinson's Disease Therapies

Parkinson's disease is a chronic disorder for which no cure is known and existing medications and therapies provide transient relief from the primary motor-related symptoms and are not disease modifying.

Levodopa (L-dopa) is used as a form of treatment. L-dopa is transformed into dopamine in the dopaminergic neurons by L-aromatic amino acid decarboxylase. However, only 1-5% of L-dopa enters the dopaminergic neurons. The remaining L-dopa is often metabolized to dopamine elsewhere, causing a wide variety of side effects. Due to feedback inhibition, L-dopa results in a reduction in the endogenous formation of L-dopa, and so eventually becomes counterproductive. Carbidopa and benserazide are dopa decarboxylase inhibitors. They help to prevent the metabolism of L-dopa before it reaches the dopaminergic neurons and are generally given as combination preparations of carbidopa/levodopa (co-careldopa) and benserazide/levodopa (co-beneldopa). Duodopa is a combination of levodopa and carbidopa.

The dopamine agonists bromocriptine, pergolide, pramipexole, ropinirole, cabergoline, apomorphine, and lisuride are moderately but only transiently effective when utilized for symptomatic treatment. Dopamine agonists can be useful for patients experiencing on-off fluctuations and dyskinesias as a result of high doses of L-dopa.

MAO-B inhibitors reduce the symptoms associated with Parkinson's disease by inhibiting the breakdown of dopamine secreted by the dopaminergic neurons.

Noradrenergic drugs such as norepinephrine may also be useful in treating Parkinson's disease.

### Diagnosis or Risk of Neurological Disease

A subject is an animal, including but not limited cows, horses, sheep, cats, dogs, pigs, horses, mice, rats, rabbits, squirrels, non-human primates, or humans. In various aspects, a subject is screened to determine if the subject is a candidate for treatment with a compound or composition described herein that can prevent or reverse a neurological disorder or inhibits, decreases, reverses, or prevents α-synuclein aggregation and fibrillation and/or aggregation. The screening methods comprise behavioral, biophysical, biochemical, and imaging assays and observations as well as questionnaires to determine if the subject is at risk for or is suffering from the early stages of a neurological disorder (e.g., Parkinson's Disease). Biophysical and behavioral observations, such as physical examination of a subject for outward symptoms of disease can be evaluated independently, or combined with questionnaires and biochemical/imaging assays. Each individual assay can also be utilized independently or combined with biophysical evaluations or other tests that are known in the art and associated with a particular neurological disorder/disease. Examples of biochemical assays include genetic screens for mutations and/or polymorphisms (e.g., SNPs analysis, short tandem repeat analysis), biomarker-based assays, protein expression assays, immunohistochemistry assays or any combinations thereof. Material for biochemical assays can be sampled from all bodily fluids and tissues. Commonly employed bodily fluids include but are not limited to blood, serum, plasma, saliva, urine, gastric and digestive fluid, tears, stool, semen, vaginal fluid, interstitial fluids derived from tumorous tissue, and cerebrospinal fluid. Methods of obtaining samples of bodily tissue and fluids include but are not limited to biopsy, cheek swabbing, nose swabbing, rectal swabbing, skin fat extraction or other collection strategies for obtaining a biological or chemical substance.

In some embodiments, screening a subject will include imaging and scanning with the use of, but not limited to PET scans, MRI scans, and STECT scans. Cardiovascular abnormalities related to Parkinson's disease in a subset of patients can be identified by heart rate spectral analysis.

In certain embodiments, a subject may be screened for early stage, development, or late-stage Parkinson's disease by screening for primary and secondary symptoms, as described herein immediately following. In other embodiments the subject may be screened for biochemical indications of disease e.g., genetic mutations and/or abnormal protein expression levels of genes and proteins, respectively, associated with a disorder, in some cases prior to any onset of symptoms such as changes in motor behavior.

Primary symptoms: There are various factors known in the art which are used to screen and diagnose a subject for various neurological disorders. For example, in one embodiment, a subject is examined to determine if the subject is suffering from Parkinson's disease by assessing presence of primary symptoms which include but are not limited to: bradykinesia, tremors, rigidity, impaired balance, or a change in gait.

Bradykinesia is slowness in voluntary movement. It produces difficulty initiating movement as well as difficulty completing movement once it is in progress. The delayed transmission of signals from the brain to the skeletal muscles, due to diminished dopamine, produces bradykinesia.

Tremors in the hands, fingers, forearm, or foot tend to occur when the limb is at rest but not when performing tasks. Tremor may occur in the mouth and chin as well.

Rigidity, or stiff muscles, may produce muscle pain and an expressionless, mask-like face. Rigidity tends to increase during movement.

Poor and impaired balance is due to the impairment or loss of the reflexes that adjust posture in order to maintain balance. Falls are common in people with Parkinson's.

Parkinsonian gait is the distinctive unsteady walk associated with Parkinson's disease. There is a tendency to lean unnaturally backward or forward, and to develop a stooped, head-down, shoulders-drooped stance. Arm swing is diminished or absent and people with Parkinson's tend to take small shuffling steps (called festination). Someone with Parkinson's may have trouble starting to walk, appear to be falling forward as they walk, freeze in mid-stride, and have difficulty making a turn.

In some embodiments, the Hoehn and Yahr staging will be used in diagnosis of primary symptoms in a subject. The Hoehn and Yahr scale is a commonly used system for describing how the symptoms of Parkinson's disease progress (Hoehn M, Yahr M (1967). “Parkinsonism: onset, progression and mortality” Neurology 17 (5): 427-42). The scale allocates stages from 0 to 5 to indicate the relative level of disability.
Stage 1: Symptoms on one side of the body only.
Stage 2: Symptoms on both sides of the body. No impairment of balance.
Stage 3: Balance impairment. Mild to moderate disease. Physically independent.
Stage 4: Severe disability, but still able to walk or stand unassisted.
Stage 5: Wheelchair-bound or bedridden unless assisted.

In other embodiments the Unified Parkinson's Disease Rating Scale (UPDRS) will be used in the diagnosis of a subject. The UPDRS is a rating tool to follow the course of Parkinson's Disease. It is made up of the 1) Mentation, Behavior, and Mood, 2) Activities of Daily Living and 3) Motor sections. These are evaluated by interview. Some sections require multiple grades assigned to each extremity. A total of 199 points are possible. 199 represents the worst (total) disability), 0—no disability.

In other embodiments the Schwab and England Activities of Daily Living assessment can be used in the diagnosis of a subject. The subject is assigned a rating from 0% to 100%. Rating can be assigned by rater or by subject.

Secondary symptoms: In some embodiments, progressive loss of voluntary and involuntary muscle control produces a number of secondary symptoms associated with Parkinson's disease (Annals of Neurology, vol. 59, No. 4, 2006). In some embodiments these symptoms are indicative of onset of primary symptoms. In other embodiments secondary symptoms can be in the absence of primary symptoms, or present with primary symptoms. These symptoms can develop well before, shortly before, during, or after the onset and development of primary symptoms. In some cases, a subject can experience and display these symptoms about 50, 40, 30, 20, 15, 10, 5, 2 years, 1 year or 6 months before or 6 months, 1, 2, 5, 10, 15, 20, 30, 40, or more years after onset and display of primary symptoms. Some patients develop these secondary symptoms well before, years before the patients develop primary symptoms characteristic with a disorder. Some secondary symptoms of Parkinson's disease include but are not limited to the following: Constipation occurring in a subject's 20's, 30's 40's or 50's; difficulty swallowing (dysphagia) saliva and food that collects in the mouth or back of the throat may cause choking, coughing, or drooling; excessive salivation (hypersalivation), excessive sweating (hyperhidrosis), loss of bladder and/or bowel control (incontinence); loss of sense of smell, olfactory dysfunction (anosmia); rapid eye movement (REM) sleep behavior disorder and other sleep disorders; changes in the cardiac sympathetic denervation, changes in the sympathetic innervation of the heart; loss of intellectual capacity (dementia), psychosocial: anxiety, depression, isolation; scaling, dry skin on the face and scalp (seborrhea); slow response to questions (bradyphrenia); small, cramped handwriting (micrographia); soft, whispery voice (hypophonia).

Therefore, in certain embodiments, diagnosis is based on symptoms and ruling out other disorders that produce similar symptoms. For example, a subject must have two or more of the primary symptoms, one of which is a resting tremor or bradykinesia. In many cases, this diagnosis is made after observing that symptoms have developed and become established over a period of time. Such diagnostic techniques described above are known in the art.

After a thorough neurological exam and medical history, the neurologist may order computerized tomography (CT scan) or magnetic resonance imaging (MRI scan) to meet the other criterion for a diagnosis of Parkinson's disease: ruling out disorders (e.g., brain tumor, stroke) that produce Parkinson's-like symptoms. Some examples follow: medications—antipsychotics (e.g., Haldol) and anti-emetics (e.g., Compazine); multiple strokes; hydrocephalus; progressive supranuclear palsy—degeneration of midbrain structures; Shy-Drager syndrome—atrophy of central and sympathetic nervous systems; Wilson's disease—copper excretion causes degeneration of the liver and basal ganglia; Blood and/or cerebrospinal fluid (CSF) analysis may be ordered to look for specific abnormalities associated with other disorders.

In some embodiments, diagnosis is based on secondary symptoms even when the subject show no or very few of the primary symptoms associated with the neurological disease.

In some embodiments, a subject that shows secondary symptoms but shows no or very few of the primary symptoms associated with the neurological disease is treated with a compound that inhibits, decreases, reverses, or prevents α-synuclein aggregation and fibrillation and/or aggregation as described. In other embodiments the subject is treated with a MAOI or a nutraceutical.

In some embodiments, the secondary symptom is selected from the group consisting of rapid eye movement sleep behavioral disorder, olfactory dysfunction, cardiac sympathetic denervation, constipation, depression, anxiety and dementia. In some embodiments, the secondary symptom is a sleep disorder.

Therefore, a compound or composition that inhibits, decreases, reverses, or prevents secondary symptoms can provide a first-line treatment for the early stages of Parkinson's disease to effectuate delayed or reduced disease progression at the first sign of the disease. Furthermore, where primary and secondary symptoms are insufficient to indicate disease onset, a genetic/biochemical or other type of screen can be conducted to determine if the subject is at risk for developing a neurological disorder (e.g., Parkinson's disease or Alzheimer's disease).

### Genetic Screening

As indicated herein above, screening a subject to determine if the subject is at-risk for developing a neurological disorder can be based on imaging, behavior, biophysical and/or biochemical screening for traits/factors associated with a neurological disorder/disease. In one such aspect of the invention, a biochemical screening involves genetic testing. Therefore, in various embodiments of the invention, a method of treating a subject at risk of a neurological disorder comprises screening genetic material from the subject to determine if markers are present (e.g., mutations of genes, expression levels of proteins) associated with the neurological disorder, thereby determining risk, and administering a compound (or a combination of two or more compounds) that inhibits, decreases, reverses, or prevents α-synuclein fibrillation and/or aggregation to the subject to delay or reduce progression of a neurological disorder. The disorder may be at the early onset stage or the subject may be entirely asymptomatic. For example, to determine if a subject is at risk for Parkinson's disease, the subject can be screened for mutations of one or more LRRK2, α-synuclein, parkin gene or a combination of two or more markers thereof. Furthermore, the subject can be screened for elevated expression levels of a protein indicative of disease onset or risk for disease. Methods of performing such genetic/biochemical screens are known in the art.

In some embodiments, the subject is screened for a mutation in a gene selected from the group consisting of leucine-rich repeat kinase 2 (LRRK2), α-synuclein (SNCA), parkin (PRKN), ubiquitin C-terminal hydrolase L1 (UCH-L1), oncogene DJ-1 gene, PTEN-induced protein kinase 1 (PINK1), and microtubule-associated protein tau (MAPT). Such mutations include but are not limited to substitution, deletion, insertion, duplication, triplication or a combination thereof.

LRRK2. In one embodiment, the subject is pre-symptomatic of primary symptoms for Parkinson's Disease, but genetic screening yields information on the presence mutations and/or polymorphisms of one or more genes associated with Parkinson's Diseases. For example, a subject is screened for the prevalence of two common leucine-rich repeat kinase 2 (LRRK2) gene mutations. In one study by Huang et al. of 830 affected patients, eight were heterozygous for the G2019S mutation, and two were heterozygous for the R1441H (4,322 G>A) mutation. Clinically, patients with LRRK2 mutations had typical levodopa responsive Parkinsonism with tremor being the most common presenting feature. Patients with the G2019S mutation had a similar age of onset of symptoms when compared with patients with other LRRK2 mutations or sporadic Parkinson's disease, although they were more likely to have a family history of Parkinson's Disease. In addition, one familial patient had a novel A1442P (4,324 G>C) mutation. Huang et al. Mov Disord. 2007 May 15: 22(7):982-9. Therefore, in one embodiment, a subject is tested to determine the presence of LRRK2 mutations and if positive for such mutations, the subject is administered one or more compounds that inhibit, decrease, reverse, or prevents α-synuclein fibrillation and/or aggregation or inhibit MAO as a prophylactic to delay, reduce or eliminate Parkinson's Disease onset or progression.

α-synuclein. In other embodiments, genetic screens detect the presence of α-synuclein gene mutations and/or polymorphisms which are major underlying genetic defects known in familial juvenile onset Parkinson's disease, and α-synuclein is a major constituent of Lewy Bodies, the pathological hallmark of Parkinson's Disease.

Evidence in the art indicates that mutant or simply over-expressed α-synuclein could cause damage by interfering with particular steps of neuronal membrane traffic. Chua and Tang J Cell Mol Med. 2006 October-December; 10(4):837-46. α-synuclein selectively blocks endoplamic reticulum (ER)-to-Golgi transport, thus causing ER stress. For example, α-synuclein toxicity could be suppressed by over-expression of the small GTPase Ypt1/Rab1, and that over-expression of the latter rescues neuron loss in invertebrate and mammalian models of α-synuclein-induced neurodegeneration (Chua and Tang, J Cell Mol Med. 2006 October-December; 10(4):837-46). α-synuclein may also serve a chaperone function for the proper folding of soluble NSF attachment receptor (SNAREs) that are important for neurotransmitter release.

Therefore in some embodiments, a subject is diagnosed or pronounced to be ‘at-risk’ after a genetic screen to determine the presence of α-synuclein mutations and/or polymorphisms and/or detection of elevated expression levels of α-synuclein, wherein mutations and/or polymorphisms and/or elevated expression levels are indicative of risk of Parkinson's Disease. Further, the subject may be optionally examined for display of one or more secondary symptoms. Thus, in one such embodiment, the subject is administered one or more compounds of the invention that inhibit, decrease, reverse, or prevent α-synuclein aggregation and fibrillation and/or aggregation or inhibit MAO as a prophylactic to delay, reduce or eliminate Parkinson's disease onset or progression.

In another embodiment, a subject is screened for LRRK2 mutations described above and α-synuclein mutations and/or polymorphisms and/or overexpression, where positive results (e.g., mutations, overexpression) are indicative of risk of developing Parkinson's Disease, and the subject is treated with one or more compounds that inhibit, decrease, reverse, or prevents α-synuclein fibrillation and/or aggregation or inhibit MAO to delay, reduce or eliminate Parkinson's disease onset or progression.

Parkin gene. In another embodiment, a subject is genetically screened to determine if one or more parkin gene mutation and/or polymorphism is present to determine risk for Parkinson's Disease. If one or more parkin gene(s) are mutated or have a polymorphism associated with a neurological disease then the subject can be treated with a compound named herein. For example, a study of 183 families affected by Parkinson's found a genetic mutation is linked to the age it affects someone. People with one mutation develop the disease 12 years earlier than average, results in Archives of Neurology show. Two mutated genes are linked with disease which starts 13 years earlier, they found. The prevalence of Parkinson's increases with age - appearing in 1% of people over 60 and 4-5% of those over 85—but it can develop in much younger patients. Research has already shown that inheriting two abnormal copies of the parkin gene is associated with the development of early-onset Parkinson's—which refers to disease which appears before the age of 50. But a team led by Massachusetts General Hospital researchers has now found that even a single mutated copy of parkin reduces the age of onset of the disease. The study was done as part of the international GenePD project, which is looking at siblings with Parkinson's disease. Families were selected for the study if affected siblings had inherited identical versions of the chromosome 6 region, which contains the parkin gene, or at least one affected member developed symptoms before the age of 54.

Among families with parkin mutations, 13 had at least one member with mutations on both copies of the gene, while in 10 families affected members had a single mutated copy of parkin. In total, 18 different mutations were found, four of which had not previously been reported. In people with parkin mutations, the average age when the disease became apparent was about 43. In a subgroup of families who had identical chromosome markers, those with no mutations in the gene developed symptoms at an average age of about 61, those with a single abnormal copy of parkin at around age 50, and those who inherited two mutated copies had onset at around age 36.

Therefore, in prophylactic treatment methods of the invention, a subject undergoes genetic screen to determine risk for Parkinson's disease (e.g., presence of one or two PRKN mutations) and if found to be at-risk, is administered one or more compound that inhibits, decreases, reverses, or prevents α-synuclein fibrillation and/or aggregation. In some further embodiments, a subject may be screened for PRKN and LRRK2 mutations and/or polymorphisms to determine if a prophylactic administration of one or more compounds described herein that inhibits, decreases, reverses, or prevents α-synuclein fibrillation and/or aggregation is desirable. In any of the genetic screens described herein, the presence of mutations and/or polymorphisms in one familial gene should not serve as exclusion criteria in a screen for further genetic variation.

In certain embodiments, a subject may be routinely screened for mutations and/or polymorphisms, to determine if at risk and determine if a prophylactic administration of one or more compounds described herein that inhibits, decreases, reverses, or prevents α-synuclein fibrillation and/or aggregation is desirable. In other embodiments, a subject may be first screened and secondary symptoms identified, determined to be at risk, and further screened for mutations and/or polymorphisms to determine if a prophylactic administration of one or more compounds described herein that inhibits, decreases, reverses, or prevents α-synuclein fibrillation and/or aggregation is desirable.

### Subjects Determined to be at Risk

Subjects asymptomatic for primary symptoms, can be selected by age, by display of one or more secondary symptoms, by genetic screening for mutations and/or polymorphisms associated with a disorder or a combination thereof, or based on a family history of the occurrence of disease, or other environmental exposures associated with the disease. For example a subject is determined to be at risk if said subject displays at least two secondary symptoms in the absence of any primary symptoms of Parkinson's disease. For example, SNPs analysis can be conducted on genetic material obtained from a subject to determine if markers associated with a particular neurological disorder are present. Such markers are known in the art. Therefore, in one example, a subject is identified as at-risk for developing a neurological disorder based on the genetic screening, and such at risk subjects can be administered a compound that inhibits, decreases, reverses, or prevents α-synuclein fibrillation and/or aggregation as a prophylactic. As discussed herein such a compound or combination of compounds include dopamine agonists, MAO inhibitors, nutraceuticals, or α-synuclein modulators such as apomorphine, apomorphine derivatives, pyrogallol, 1,4-naphthoquinone, cisplatin, isoproterenol, pyrogallin, cianidanol, sulfasalazine, quinalizarin, benserazide, hexachlorophene, pyrvinium pamoate, dobutamine, methyl-dopa, curcumin, berberine chloride, daidzein, merbromin, norepinephrine, dopamine hydrochloride, carbidopa, ethylnorepinephrine hydrochloride, tannic acid, elaidyphosphocholine, hydroquinone, chlorophyllide Cu complex Na salt, methyldopa, isoproterenol hydrochloride, benserazide hydrochloride, dopamine, dobutamine hydrochloride, thyroid hormone, purpurin, sodium beta-nicotinamide adenine dinucleotide phosphate, lansoprazole, dyclonine hydrochloride, pramoxine hydrochloride, azobenzene, cefamandole sodium, cephaloridine, myricetin, 6,2′,3′-trihydroxyflavone, 5,7,3′,4′,5′-pentahydroxyflavone, 7,3′,4′,5′-tetrahydroxyflavone, (5,6,7,4′-tetrahydroxyflavone), baicalein, eriodictyol, 7,3′,4′-trihydroxyisoflavone, epigallocatechin gallate, quercetin, gossypetin (3,5,7,8,3′,4′-hexahydroxyflavone), 2′,3′-dihydroxyflavone, 3′,4′-dihydroxyflavone, 5,6-dihydroxy-7-methoxyflavone, baicalein-7-methyl ether, 1-dopa, DOPAC, homogentisic acid, 6-hydroxydopamine, epinephrine, 3,4-dihydroxycinnamic acid, 2,3-dihydroxynaphthalene, 3,4-dihydroxybenzoic acid, 3,4,5-trihydroxybenzoic acid, 1,2,3-trihydroxybenzoic acid, gallate (gallic acid), benzoquinone, catechol, rifampicin, rosmarinic acid, baicalin, tanshinones I and II, emodin, procyanidin B4, resveratrol, rutin, fisetin, luteolin, fustin, epicatechin gallate, catechin, alizarin, tannic acid, eriodyctol, carboplatin, purpurogallin-4-carboxylic acid, koparin, 2,3,4-trihydroxy-4′-ethexybenzophenone, baeomycesic acid, hamtoxylin, iriginol hexaaceatate, 4-acetoxyphenol, theaflavin monogallate, theaflavin digallate, stictic acid, purpurogallin, 2,5-dihydroxy-3,4-dimethoxy-4′-ethoxybenzophenone, promethazine hydrochloride, oxidopamine hydrochloride, pyrantel pamoate, elaidylphosphocholine, amphotericin B, gallic acid, fumarprotocetraric acid, theaflavin, haematoxylin pentaacetate, 4-methoxydalbergione, epigallocatechin-3-monogallate, rolitetracycline, 7,3′-dimethoxyflavone, liquiritigenin dimethyl ether, catechin pentaacetate, apigenin, 3,4-dedesmethyl-5-deshydroxy-3′-ethoxyscleroin, derivatives thereof, or a combination thereof.

In some embodiments, a subject is at risk if the subject displays at least 2, at least 3, at least 4, at least 5, or more of the secondary symptoms. The subject can display the at least 2,3, 4, 5, or more symptoms concurrently, simultaneously, consecutively, in a non-overlapping time frame, separated by hours, separated by days, separated by weeks, separated by months, or separated by years. In some embodiments, a subject is at risk if a subject displays at least 1, at least 2, at least 3, or more secondary symptoms in combination with expression of a known genetic marker or protein biomarker of disease. In some embodiments the genetic screening is prior to the onset of secondary symptoms. In other embodiments the genetic screening is following the display of at least one secondary symptom. In other embodiments, a subject is at risk if there is a family history of disease, such as a mother, father, brother, sister, son, daughter, aunt, uncle, or grandparent, with or without an accompanying genetic screening. In some embodiments a subject is at risk of disease if the subject is exposed to a known environmental trigger or toxin that is know to cause, or effect classic disease or disease-like symptoms.

### Subjects, Compounds, and Compositions

In various embodiments of the invention, dosage forms of a compound that prevents the onset of a neurological disease, or a compound that inhibits, decreases, reverses, or prevents α-synuclein fibrillation and/or aggregation (alone or in combination with other agents) are administered to a subject. A subject is an animal, including but not limited cows, horses, sheep, cats, dogs, pigs, horses, mice, rats, rabbits, squirrels, non-human primates, and humans. In certain embodiments, the subject is human. Furthermore, in any of such subjects, the disorder may be at the early stage or the subject may be entirely asymptomatic.

Subjects: In various embodiments, a subject is of a particular age, at which time dosage forms of one or more compounds described herein are administered which inhibit, decrease, reverse, or prevents α-synuclein fibrillation and/or aggregation. For example, a human subject to whom a compound that inhibits, decreases, reverses, or prevents α-synuclein fibrillation and/or aggregation (as described herein) is administered can be from 5 to 15, 10 to 25, 20 to 35, 25 to 45, 30 to 50, 40 to 60, 50 to 75, 60 to 90 years old or more. In some embodiments, a subject treated with a compound that inhibits, decreases, reverses, or prevents α-synuclein fibrillation and/or aggregation is of a pre-selected age, but does not exhibit primary symptoms associated with a neurological disorder. Therefore, a pre-symptomatic (or α-symptomatic) subject can be administered one or more compounds which inhibit, decrease, reverse, or prevents α-synuclein fibrillation and/or aggregation as a prophylactic method to reduce or eliminate symptoms associated with disease, and/or delay or reduce disease(e.g., Parkinson's disease) progression. In other embodiments a subject is determined to be ‘at-risk’ and the subject can be administered one or more compounds which inhibit, decrease, reverse, or prevents α-synuclein fibrillation and/or aggregation as a prophylactic method to reduce or eliminate symptoms associated with disease, and/or delay or reduce disease(e.g., Parkinson's Disease) progression.

Compounds: In one embodiment, the invention provides compositions comprising compounds named herein or their derivatives that prevent or delay the progression of a neurological disorder. In some embodiments the invention provides compositions comprising compounds named herein or their derivatives that inhibit, reverse, or prevent α-synuclein mediated aggregation or fibrillation and/or aggregation. In some embodiments the compound is selected from the group consisting of apomorphine, apomorphine derivatives, pyrogallol, 1,4-naphthoquinone, cisplatin, isoproterenol, pyrogallin, cianidanol, sulfasalazine, quinalizarin, benserazide, hexachlorophene, pyrvinium pamoate, dobutamine, methyl-dopa, curcumin, berberine chloride, daidzein, merbromin, norepinephrine, dopamine hydrochloride, carbidopa, ethylnorepinephrine hydrochloride, tannic acid, elaidyphosphocholine, hydroquinone, chlorophyllide Cu complex Na salt, methyldopa, isoproterenol hydrochloride, benserazide hydrochloride, dopamine, dobutamine hydrochloride, thyroid hormone, purpurin, sodium beta-nicotinamide adenine dinucleotide phosphate, lansoprazole, dyclonine hydrochloride, pramoxine hydrochloride, azobenzene, cefamandole sodium, cephaloridine, myricetin, 6,2′,3′-trihydroxyflavone, 5,7,3′,4′,5′-pentahydroxyflavone, 7,3′,4′,5′-tetrahydroxyflavone, (5,6,7,4′-tetrahydroxyflavone), baicalein, eriodictyol, 7,3′,4′-trihydroxyisoflavone, epigallocatechin gallate, quercetin, gossypetin (3,5,7,8,3′,4′-hexahydroxyflavone), 2′,3′-dihydroxyflavone, 3′,4′-dihydroxyflavone, 5,6-dihydroxy-7-methoxyflavone, baicalein-7-methyl ether, 1-dopa, DOPAC, homogentisic acid, 6-hydroxydopamine, epinephrine, 3,4-dihydroxycinnamic acid, 2,3-dihydroxynaphthalene, 3,4-dihydroxybenzoic acid, 3,4,5-trihydroxybenzoic acid, 1,2,3-trihydroxybenzoic acid, gallate (gallic acid), benzoquinone, catechol, rifampicin, rosmarinic acid, baicalin, tanshinones I and II, emodin, procyanidin B4, resveratrol, rutin, fisetin, luteolin, fustin, epicatechin gallate, catechin, alizarin, tannic acid, eriodyctol, carboplatin, purpurogallin-4-carboxylic acid, koparin, 2,3,4-trihydroxy-4′-ethexybenzophenone, baeomycesic acid, hamtoxylin, iriginol hexaaceatate, 4-acetoxyphenol, theaflavin monogallate, theaflavin digallate, stictic acid, purpurogallin, 2,5-dihydroxy-3,4-dimethoxy-4′-ethoxybenzophenone, promethazine hydrochloride, oxidopamine hydrochloride, pyrantel pamoate, elaidylphosphocholine, amphotericin B, gallic acid, fumarprotocetraric acid, theaflavin, haematoxylin pentaacetate, 4-methoxydalbergione, epigallocatechin-3-monogallate, rolitetracycline, 7,3′-dimethoxyflavone, liquiritigenin dimethyl ether, catechin pentaacetate, apigenin, 3,4-dedesmethyl-5-deshydroxy-3′-ethoxyscleroin, derivatives thereof, or a combination thereof. In some embodiments the invention provides compounds or compositions having therapeutic utility for a known disease, in some cases Parkinson's disease, but have not been utilized or considered for α-synuclein for the inhibition, reversal or prevention of α-synuclein aggregation and fibrillation and/or aggregation. In other embodiments the invention provides for compounds or compositions have not been considered for prophylactic or disease-inhibiting, disease-reversing, disease-modifying, or disease-prevention uses.

In some embodiments, a compound that inhibits, decreases, reverses, or prevents α-synuclein fibrillation and/or aggregation is co-administered with another therapeutic agent. In other embodiments a compound such as a MAOI is administered with an α-synuclein-modulating compound. In other embodiments a MAOI is administered prior to the onset of any primary symptoms. “Co-administration,” “administered in combination with,” and their grammatical equivalents, as used herein, encompasses administration of two or more agents to a subject so that both agents and/or their metabolites are present in the subject at the same time. Co-administration includes simultaneous administration in separate compositions, administration at different times in separate compositions, or administration in a composition in which both agents are present. In some embodiments, the compositions of the invention include an agent such as levodopa. In some embodiments the compositions of the invention include an agent such carbidopa, which blocks the conversion of levodopa to dopamine in the blood. In some embodiments, the compositions of the invention include a COMT inhibitor such as entacapone to inhibit the decrease of levodopa absorption and efficacy or to inhibit the metabolism of levodopa to 3-O-methyldopa (3-OMD). In some embodiments, the compositions of the invention include a monoamine oxidase type B (MAO-B) inhibitor such as selegiline. In some embodiments, the compositions of the invention include amantadine.

In some embodiments, the compositions of the invention are utilized to inhibit, reverse, prevent or decrease the progression of a neurological disorder. In some embodiments, the progression of a neurological disorder is delayed by an average of at least about 5, 10, 15, 20, 25, 30, 35, 40, 45, 50, 55, 60, 65, 70, 75, 80, 85, 90, 95, or more than 95%, compared to the progression without the compositions described herein. In some embodiments, the progression of a neurological disorder is delayed by an average of at least about 5%, compared to the progression without the compositions described herein. In some embodiments, the progression of a neurological disorder is delayed by an average of at least about 10%, compared to the progression without the compositions described herein. In some embodiments, the progression of a neurological disorder is delayed by an average of at least about 15%, compared to the progression without compositions described herein. In some embodiments, the progression of a neurological disorder is delayed by an average of at least about 20%, compared to the progression without the compositions described herein. In some embodiments, the progression of a neurological disorder is substantially stopped. “Substantially stopped” as used herein encompasses no measurable or no statistically significant progression of the neurological disorder, when the compositions of the invention are administered. In some embodiments the neurological disorder is Parkinson's disease.

In some embodiments, the invention provides a composition that contains a compound that inhibits, decreases, reverses, or prevents α-synuclein fibrillation and/or aggregation. In other embodiments the compound can inhibit MAO. In some embodiments, the concentration of the is less than 100%, 90%, 80%, 70%, 60%, 50%, 40%, 30%, 20%, 19%, 18%, 17%, 16%, 15%,14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v.

In some embodiments, the concentration of the compound that inhibits, decreases, reverses, or prevents α-synuclein fibrillation and/or aggregation or inhibits MAO is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 1.25%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of the compound that inhibits, decreases, reverses, or prevents α-synuclein fibrillation and/or aggregation or inhibits MAO is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v. v/v.

In some embodiments, the concentration of the compound that inhibits, decreases, reverses, or prevents α-synuclein fibrillation and/or aggregation or inhibits MAO is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, a concentration of the compound that inhibits, decreases, reverses, or prevents α-synuclein fibrillation and/or aggregation or inhibits MAO is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the concentration of the compound that inhibits, decreases, reverses, or prevents α-synuclein fibrillation and/or aggregation or inhibits MAO is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.65 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

In some embodiments, a concentration of the compound that inhibits, decreases, reverses, or prevents α-synuclein fibrillation and/or aggregation or inhibits MAO is in the range of 0.0001-10 g, 0.0005-9 g, 0.001-8 g, 0.005-7 g, 0.01-6 g, 0.05-5 g, 0.1-4 g, 0.5-4 g, or 1-3 g.

In exemplary embodiments, compositions of the invention include a compound that inhibits, decreases, reverses, or prevents α-synuclein fibrillation and/or aggregation or inhibits MAO, where the compound is present in an amount from about 0.001-1000 mg, or about 0.010-1000 mg, or about 0.050-1000 mg, or about 0.100-1000 mg, or about 1-500 mg, or about 1-100 mg, or about 1-50 mg, or about 0.100-500 mg, or about 0.100-100 mg, or about 0.200-100 mg, or about 0.200-50 mg, or about 0.1 mg, or about 1 mg, or about 5 mg, or about 10 mg, or about 15 mg, or about 20 mg, or about 50 mg, or about 100 mg, or about 200 mg, or about 500 mg, or about 600 mg, or about 700 mg, or about 800 mg, or about 900 mg, or about 1000 mg.

In liquid preparations, the compound that inhibits, decreases, reverses, or prevents α-synuclein fibrillation and/or aggregation or inhibits MAO can be present at about 0.001-1000 mg/ml, or 0.001-500 mg/ml, or 0.001-200 mg/ml, or about 0.001, 0.01, 0.1, 1, 5, 10, 20, 50, 50, 100, 500 or 1000 mg/ml. At higher levels of the compound, solubility can be enhanced by adjusting the type of diluent.

In some embodiments, the compound that inhibits, decreases, reverses, or prevents α-synuclein fibrillation and/or aggregation or inhibits MAO is co-administered in combination with another agent. In some embodiments, a molar ratio of one or more of the compound that inhibits, decreases, reverses, or prevents α-synuclein fibrillation and/or aggregation or inhibits MAO to the other agent, e.g. levodopa can be 0.0001:1 to 1:1. Without limiting the scope of the invention, the molar ratio of the compound that inhibits, decreases, reverses, or prevents α-synuclein fibrillation and/or aggregation or inhibits MAO to the other agent can be about 0.0001:1 to about 10:1, or about 0.001:1 to about 5:1, or about 0.01:1 to about 5: 1, or about 0.1:1 to about 2:1, or about 0.2:1 to about 2:1, or about 0.5:1 to about 2:1, or about 0.1:1 to about 1:1.

Without limiting the scope of the present invention, the molar ratio the compound that inhibits, decreases, reverses, or prevents α-synuclein fibrillation and/or aggregation or inhibits MAO to the other agent can be about 0.03×10-5:1, 0.1×10-5:1, 0.04×10-3:1, 0.03×10-5:1, 0.02×10-5:1, 0.01×10-3:1, 0.1×10-3:1, 0.15×10-3:1, 0.2×10-3:1, 0.3×10-3:1, 0.4×10-3:1, 0.5×10-3:1, 0.15×10-2:1, 0.1×10-2:1, 0.2×10-2:1, 0.3×10-2:1, 0.4×10-2:1, 0.5×10-2:1, 0.6×10-2:1, 0.8×10-2:1, 0.01:1, 0.1:1; or 0.2:1 per dose.

Without limiting the scope of the present invention, the molar ratio of the compound that inhibits, decreases, reverses, or prevents α-synuclein fibrillation and/or aggregation to the other agent can be about 0.001:1, 0.002:1, 0.003:1, 0.004:1, 0.005:1, 0.006:1, 0.007:1, 0.008:1, 0.009:1, 0.01:1, 0.02:1, 0.03:1, 0.04:1, 0.05:1, 0.06:1, 0.07:1, 0.08:1, 0.09:1, 0.1:1, 0.2:1, 0.3:1, 0.4:1, 0.5:1, 0.6:1, 0.7:1, 0.8:1, 0.9:1, 1:1, 2:1, 3:1, 4:1, or 5:1 per dose.

Pharmaceutical Compositions: In some embodiments the invention provides pharmaceutical compositions that further include a pharmaceutically acceptable excipient. In some embodiments, the pharmaceutical compositions are suitable for oral administration. In some embodiments, the pharmaceutical compositions are suitable for transdermal administration. In some embodiments, the pharmaceutical compositions are suitable for injection. Other forms of administration are also compatible with embodiments of the pharmaceutical compositions of the invention, as described herein.

The pharmaceutical compositions of the invention may be administered in dosages as described herein (see, e.g., Compositions). When the compound that inhibits, decreases, reverses, or prevents α-synuclein fibrillation and/or aggregation or inhibits MAO and the agents that are used in combination, both components may be mixed into a preparation or both components may be formulated into separate preparations to use them in combination separately or at the same time.

This invention therefore provides pharmaceutical compositions that contain, as the active ingredient, a compound that inhibits, decreases, reverses, or prevents α-synuclein fibrillation and/or aggregation or inhibits MAO, or a pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

This invention further provides pharmaceutical compositions that contain, as the active ingredient, a compound that inhibits, decreases, reverses, or prevents α-synuclein fibrillation and/or aggregation or inhibits MAO, or a pharmaceutically acceptable salt and/or coordination complex thereof, another therapeutic agent or a pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

The agent and/or compound that inhibits, decreases, reverses, or prevents α-synuclein fibrillation and/or aggregation or inhibits MAO may be prepared into pharmaceutical compositions in dosages as described herein (see, e.g., Compositions). Such compositions are prepared in a manner well known in the pharmaceutical art.

Pharmaceutical compositions for oral administration. In some embodiments, the invention provides a pharmaceutical composition for oral administration containing a compound that inhibits, decreases, reverses, or prevents α-synuclein fibrillation and/or aggregation or inhibits MAO, and a pharmaceutical excipient suitable for oral administration. In some embodiments, the invention provides a pharmaceutical composition for oral administration containing a combination of a compound that inhibits, decreases, reverses, or prevents α-synuclein fibrillation and/or aggregation or inhibits MAO, another agent, and a pharmaceutical excipient suitable for oral administration.

In some embodiments, the invention provides a solid pharmaceutical composition for oral administration containing: (i) an effective amount of an agent; (ii) an effective amount of a compound that inhibits, decreases, reverses, or prevents α-synuclein fibrillation and/or aggregation or inhibits MAO capable of delaying or stopping the progression of a neurological disorder; and (iii) a pharmaceutical excipient suitable for oral administration.

In some embodiments, the composition further contains: (iv) an effective amount of a second agent. In some embodiments, the composition further contains: (iv) an effective amount of an agent such as levodopa. In some embodiments, the composition further contains: (iv) an effective amount of an agent such carbidopa, which blocks the conversion of levodopa to dopamine in the blood. In some embodiments, the composition further contains: (iv) an effective amount of a COMT Inhibitor, such as entacapone. In some embodiments, the composition further contains: (iv) an effective amount of a monoamine oxidase type B (MAO-B) inhibitor such as selegiline. In some embodiments, the composition further contains: (iv) an effective amount of amantadine. In some embodiments, the composition further contains: (iv) an effective amount of nutraceutical.

In some embodiments, the pharmaceutical composition may be a liquid pharmaceutical composition suitable for oral consumption.

In some embodiments, the invention provides a solid pharmaceutical composition for oral administration containing a compound named herein at about 0.10-1000 mg, and a pharmaceutically acceptable excipient. In some embodiments, the invention provides a liquid pharmaceutical composition for oral administration containing a compound named herein at about 0.001-1000 mg/ml, and a pharmaceutically acceptable excipient.

Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

This invention further encompasses anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the essential active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10.

However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, preferred ionic surfactants include, by way of example: lecithins, lysolecithin, phospholipids, lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of a compound named herein and/or another therapeutic agent and to minimize precipitation of the compound and/or another therapeutic agent. This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG ; amides and other nitrogen-containing compounds such as 2-pyrrolidone, 2-piperidone, .epsilon.-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, disopropylethylamine, ethanolamine, ethylenediamine, triethanolamine, triethylamine, trisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Examples may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

Pharmaceutical compositions for injection In some embodiments, the invention provides a pharmaceutical composition for injection containing a compound that inhibits, decreases, reverses, or prevents α-synuclein fibrillation and/or aggregation, inhibits MAO and a pharmaceutical excipient suitable for injection. Components and amounts of agents in the compositions are as described herein.

In some embodiments the invention provides a pharmaceutical composition for injection containing a combination of a compound named herein and an agent, and a pharmaceutical excipient suitable for injection. Components and amounts of agents in the compositions are as described herein.

The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating a compound that inhibits, decreases, reverses, or prevents α-synuclein fibrillation and/or aggregation, inhibits MAO and/or another compound in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Pharmaceutical compositions for topical (e.g., transdermal) delivery In some embodiments, the invention provides a pharmaceutical composition for transdermal delivery containing a compound that inhibits, decreases, reverses, or prevents α-synuclein fibrillation and/or aggregation or inhibits MAO and a pharmaceutical excipient suitable for transdermal delivery. In some embodiments, the invention provides a pharmaceutical composition for transdermal delivery containing a combination of a compound that inhibits, decreases, reverses, or prevents α-synuclein fibrillation and/or aggregation or inhibits MAO and another agent, and a pharmaceutical excipient suitable for transdermal delivery. Components and amounts of the compound and other agents in the compositions are as described herein.

Compositions of the present invention can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another formulation for use in the methods of the present invention employs transdermal delivery devices (e.g. patches or minipumps). Such transdermal devices may be used to provide continuous or discontinuous infusion of the compounds named herein in controlled amounts, either with or without another agent. Thus, in some embodiments the invention provides a transdermal device incorporating a compound named herein. In some embodiments the invention provides a transdermal device incorporating a compound in combination with another agent, e.g. levodopa.

The construction and use of transdermal devices for the delivery of pharmaceutical agents is well known in the art. See, e.g., U.S. Pat. Nos. 5,023,252, 4,992,445 and 5,001,139. Such devices may be constructed for continuous, pulsatile, or on demand delivery of pharmaceutical agents.

Pharmaceutical compositions for inhalation. Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

Other pharmaceutical compositions: Pharmaceutical compositions may also be prepared from compositions described herein and one or more pharmaceutically acceptable excipients suitable for nasal (nasal puff), intracranial (slow release with the aid of an implanted device), sublingual, buccal, rectal, intraosseous, intraocular, intranasal, epidural, or intraspinal administration. Preparations for such pharmaceutical compositions are well-known in the art. See, e.g., See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

Kits: The invention also provides kits. The kits include a compound that inhibits, decreases, reverses, or prevents α-synuclein fibrillation and/or aggregation or a MAO inhibitor, in suitable packaging, and written material that can include instructions for use, discussion of clinical studies, listing of side effects, and the like. The kit may further contain another agent, e.g. levodopa or nicotine. In some embodiments, the compound and the other agent are provided as separate compositions in separate containers within the kit. In some embodiments, the compound and the other agent are provided as a single composition within a container in the kit. Suitable packaging and additional articles for use (e.g., measuring cup for liquid preparations, foil wrapping to minimize exposure to air, and the like) are known in the art and may be included in the kit. The kit may further include materials to test for the predisposition of a neurological disorder, e.g. Parkinson's disease.

### Administration

The methods involve the administration of a compound that inhibits, decreases, reverses, or prevents α-synuclein fibrillation and/or aggregation or inhibits MAO, e.g., to delay or stop the progression of a neurological disorder. In some embodiments, another agent that produces a side effect is administered in combination with the compound. When two or more agents are co-administered, they may be co-administered in any suitable manner, e.g., as separate compositions, in the same composition, by the same or by different routes of administration.

In some embodiments, a compound named herein is administered in a single dose. Typically, such administration will be by injection, e.g., intravenous or subcutaneous injection. However, other routes may be used as appropriate. A single dose of a compound may also be used when it is administered with other agent (e.g., a dopaminergic agent such as levodopa) for treatment of an acute condition. Alternatively chronic dosing of a compound named herein may be administered with an agent used for treatment of an acute condition.

In some embodiments, a compound that inhibits, decreases, reverses, or prevents α-synuclein fibrillation and/or aggregation or inhibits MAO is administered in multiple doses. Dosing may be about once, twice, three times, four times, five times, six times, or more than six times per day. Dosing may be about once a month, once every two weeks, once a week, or once every other day. In another embodiment the administration of the compound continues for less than about 7 days. In yet another embodiment the administration continues for more than about 6, 10, 14, 28 days, two months, six months, or one year. In some cases, continuous dosing is achieved and maintained as long as necessary, including the rest of a subject life. In some embodiments, the compound that inhibits, decreases, reverses, or prevents α-synuclein fibrillation and/or aggregation is administered continually, e.g. with a minipump, patch or stent.

A compound that inhibits, decreases, reverses, or prevents α-synuclein fibrillation and/or aggregation can be administered via subcutaneous injection using a small pump which is carried by the patient. A low dose is automatically administered throughout the day, reducing the fluctuations of motor symptoms by providing a steady dose of dopaminergic stimulation. The injection site can be changed daily and rotated around the body to avoid the formation of nodules. Compounds that inhibit, decrease, reverse, or prevent α-synuclein fibrillation and/or aggregation can be administered in a more acute dose as an autoinjector pen for emergency doses such as after a fall or first thing in the morning.

In some embodiments, a compound that inhibits, decreases, reverses, or prevents α-synuclein fibrillation and/or aggregation or inhibits MAO is administered in combination with another agent. In another embodiment the compound and the other agent are administered together about once per day to about 6 times per day. In another embodiment the administration of the compound that inhibits, decreases, reverses, or prevents α-synuclein fibrillation and/or aggregation or inhibits MAO and the other agent continues for less than about 7 days. In yet another embodiment the administration continues for more than about 6, 10, 14, 28 days, two months, six months, or one year. In some cases, continuous dosing is achieved and maintained as long as necessary, including the rest of a subject life. In some embodiments, administration is continuous, e.g. with a minipump, patch or stent.

Administration of a compound that inhibits, decreases, reverses, or prevents α-synuclein fibrillation and/or aggregation or a MAO inhibitor may continue as long as necessary. In some embodiments, the compound is administered for more than 1, 2, 3, 4, 5, 6, 7, 14, 28 days or 1 year. In some embodiments, the compound is administered for less than 28, 14, 7, 6, 5, 4, 3, 2, or 1 day. In some embodiments, the compound is administered chronically on an ongoing basis, e.g., for the treatment of chronic effects.

An effective amount of a compound that inhibits, decreases, reverses, or prevents α-synuclein fibrillation and/or aggregation or inhibits MAO and/or an effective amount of another agent may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent.

Compounds that inhibit, decrease, reverse, or prevent α-synuclein fibrillation and/or aggregation or inhibit MAO and/or the other agent may be administered in dosages as described herein (see, e.g., Compositions). Dosing ranges for the agents as described herein are known in the art. It is also known in the art that due to intersubject variability, pharmacokinetics, individualization of dosing regimen is necessary for optimal therapy. Dosing for the compound that may be found by routine experimentation. Daily dose range may depend on the form of the compound that and/or factors with which the compound is administered, as described herein.

When a compound that inhibits, decreases, reverses, or prevents α-synuclein fibrillation and/or aggregation or inhibits MAO is administered in a composition that comprises one or more and the agent has a shorter half-life than the compound, unit dose forms of the agent and the compound that may be adjusted accordingly.

### Combination Treatment

In various aspects of the invention, a subject at risk or suffering from a neurological disorder is administered a compound that inhibits, decreases, reverses, or prevents α-synuclein fibrillation and/or aggregation or inhibits MAO as described herein in combination with one, two, three, four or five additional active agents (e.g., drugs). Such additional agents include but are not limited to anti-emetic, 1-dihydroxyphenylalanine, aromatic acid decarboxylase inhibitor, catechol-O-methyltransferase inhibitor, MAOB inhibitor, nutraceuticals, a different dopamine agonist, a vitamin, tocopherol, otigotine, lisuride, nicotinic receptor agonist, amantadine, carbidopa, entacapone, levodopa, bromocriptine, pergolide, pramixpexole, cabergoline or ropinorole.

In various embodiments, a compound that inhibits, decreases, reverses, or prevents α-synuclein fibrillation and/or aggregation or inhibits mao is administered in combination with one or more dopamine agonists, which include but are not limited to bromocriptine, pergolide, pramipexole, ropinirole , cabergoline, apomorphine, and lisuride. Dopamine agonists initially act by stimulating some of the dopamine receptors. Dopamine agonists can be useful for patients experiencing on-off fluctuations and dyskinesias as a result of high doses of Levodopa.

In one aspect, the invention provides compositions and methods to reduce or eliminate the effects of a dopaminergic agent. In some embodiments, the compositions and methods retain or enhance a desired effect of the dopaminergic agent, e.g., anti-parkinsonian effect. The methods and compositions of the invention apply to any dopaminergic agent for which it is desired to reduce one or more side effects. In some embodiments, the compositions and methods of the invention utilize a dopamine precursor. In some embodiments, the compositions and methods of the invention utilize a dopamine agonist. In some embodiments, the dopaminergic agent is levodopa, bromocriptine, pergolide, pramipexole, cabergoline, ropinorole, or a combination thereof. In some embodiments, the dopaminergic agent is levodopa. In some embodiments, the compositions and methods of the invention utilize one or more agents used in the art in combination with a dopamine agent treatment to achieve a therapeutic effect. For instance, in one exemplary embodiment the compositions and methods of the invention utilize levodopa in combination with an agent such as carbidopa, which blocks the conversion of levodopa to dopamine in the blood. In another exemplary embodiment, the compositions and methods of the invention utilize levodopa in combination with a COMT Inhibitor, such as entacapone. In another exemplary embodiment, the compositions and methods of the invention utilize levodopa in combination with a monoamine oxidase type B (MAO-B) inhibitor such as selegiline. In yet another exemplary embodiment, the compositions and methods of the invention utilize levodopa in combination with amantadine.

In some embodiments, the invention provides compositions and methods utilizing a combination of a dopaminergic agent and a nicotinic receptor modulator that reduces or eliminates a side effect associated with dopaminergic agent treatment. Typically, the nicotinic receptor modulator is an agonist. In some embodiments, the nicotinic receptor agonist modulates a nicotinic receptor comprising at least one cc subunit or a nicotinic receptor containing at least one α subunit and at least one β subunit. In some embodiments, the α subunit is selected from the group consisting of α2, α3, α4, α5, α6, α7, α8, α9, and α10 and the β subunit of β2, β3 and β4. In some embodiments, the nicotinic receptor agonist modulates a nicotinic receptor composed of subunits selected from the group consisting of α4β2, α6β2, α4α6β2, α4α5β2, α4α6β2β3, α6β2β3 and α4α2β2.

In some embodiments, the dopaminergic agents include a dopamine precursor or a dopamine receptor agonist Examples of dopaminergic agents include, but are not limited to, levodopa, bromocriptine, pergolide, pramipexole, cabergoline, ropinorole, apomorphine or a combination thereof.

The nicotinic receptor modulator causing a decrease in the side effects of the dopaminergic agent may be an agonist or an antagonist of a protein. The modulatory effect may be dose-dependent, e.g., some modulators act as agonists in one dosage range and antagonists in another. In some embodiments, a modulator of a nicotinic receptor is used in a dosage wherein it acts primarily as an agonist.

Typically, the use of the nicotinic receptor modulator, e.g., agonist, results in a decrease in one or more side effects of the dopaminergic agent. The therapeutic effect(s) of the dopaminergic agent may be decreased, remain the same, or increase; however, in preferred embodiments, if the therapeutic effect is decreased, it is not decreased to the same degree as the side effects. It will be appreciated that a given dopaminergic agent may have more than one therapeutic effects and or one or more side effects, and it is possible that the therapeutic ratio (in this case, the ratio of change in desired effect to change in undesired effect) may vary depending on which effect is measured. However, at least one therapeutic effect of the dopaminergic agent is decreased to a lesser degree than at least one side effect of the dopaminergic agent.

In addition, in some embodiments, one or more therapeutic effects of the dopaminergic agent are enhanced by use in combination with a nicotinic receptor modulator, while one or more side effects of the dopaminergic agent is reduced or substantially eliminated. For example, in some embodiments, the anti-parkinsonian effect of the dopaminergic agent is enhanced while one or more side effects of the dopaminergic agent is reduced or substantially eliminated.

Hence, in some embodiments the invention provides compositions that include a dopaminergic agent and a nicotinic receptor modulator, where the dopaminergic agent is present in an amount sufficient to exert a therapeutic effect and the nicotinic receptor modulator is present in an amount sufficient to decrease side effect of the dopaminergic agent when compared to the side effect without the nicotinic receptor modulator, when the composition is administered to an animal.

In some embodiments, compositions of the invention include one or more dopaminergic agent with one or more nicotinic receptor modulators. One or more of the dopaminergic agent may have one or more side effects which are desired to be decreased. In some embodiments, compositions of the invention include one or more agents, one or more dopaminergic agent with one or more nicotinic receptor modulators. The one or more agents are agents used in the art in combination with a dopamine agent treatment to achieve a therapeutic effect and/or reduce a side effect. In some embodiments, the compositions of the invention include an agent such as carbidopa, which blocks the conversion of levodopa to dopamine in the blood. In some embodiments, the compositions of the invention include a COMT Inhibitor, such as entacapone. In some embodiments, the compositions of the invention include a monoamine oxidase type B (MAO-B) inhibitor such as selegiline. In some embodiments, the compositions of the invention include amantadine.

Levodopa. an aromatic amino acid, is a white, crystalline compound, slightly soluble in water, with a molecular weight of 197.2. It is designated chemically as (−)-L-a-amino-b-(3,4-dihydroxybenzene)propanoic acid. Its empirical formula is C₉H₁₁NO₄, and its structural formula is

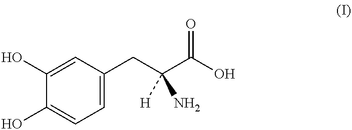

Levodopa is used for the treatment of Parkinson's disease. Parkinson's disease is a progressive, neurodegenerative disorder of the extrapyramidal nervous system affecting the mobility and control of the skeletal muscular system. Its characteristic features include resting tremor, rigidity, and bradykinetic movements

Current evidence indicates that symptoms of Parkinson's disease are related to depletion of dopamine in the corpus striatum. Administration of dopamine is ineffective in the treatment of Parkinson's disease apparently because it does not cross the blood-brain barrier. However, levodopa, the metabolic precursor of dopamine, does cross the blood-brain barrier, and presumably is converted to dopamine in the brain. This is thought to be the mechanism whereby levodopa relieves symptoms of Parkinson's disease.

In one embodiment, a compound that inhibits, decreases, reverses, or prevents α-synuclein fibrillation and/or aggregation or inhibits MAO is administered with levodopa and an antiemetic to reduce side effects associated with levodopa/the compound. For various embodiments, administration of combination drugs can be in a single multi-drug dosage form, or can comprise administration of multiple dosage forms for each drug. In such multiple dosage forms, a first drug, second drug or third drug can be administered concurrently or at different time points. For example, in one embodiment of a multiple dosage form administration, an antiemetic is administered first to prime the subject's body, followed by administration of the compound and levodopa. In this way, the subject's body has absorbed the antiemetic and is less likely to show adverse side effects associated with anti-parkinsonian drugs.

In one embodiment, a subject is administered a compound that inhibits, decreases, reverses, or prevents α-synuclein fibrillation and/or aggregation or inhbits MAO and at least one other agent that is anti-emetic, 1-dihydroxyphenylalanine, aromatic acid decarboxylase inhibitor, catechol-O-methyltransferase inhibitor, monoamine oxidase-B inhibitor, a different dopamine agonist, otigotine, lisuride, nicotinic receptor agonist, amantadine, carbidopa, entacapone, levodopa, bromocriptine, pergolide, pramixpexole, cabergoline or ropinorole.

In various embodiments described herein above, administration of a compound that inhibits, decreases, reverses, or prevents α-synuclein fibrillation and/or aggregation alone or in combination of other agents results in reduced side effects that would otherwise be present if the compound was absent. Therefore, in one aspect of the invention, administration of the compound results in reduced side effects.

For example, the principal adverse reactions of dopaminergic agent include headache, diarrhea, hypertension, nausea, vomiting, involuntary movements (e.g. dyskinesias), mental disturbances, depression, syncope, hallucinations, and abnormal renal function.

The invention provides compositions and methods utilizing a nicotinic receptor modulator that reduces or eliminates a side effect associated with dopaminergic agent treatment. In some embodiments, the invention provides compositions and methods utilizing a nicotinic receptor modulator that reduces or eliminates dyskinesias associated with dopaminergic agent treatment. Without being limited to any theory, one possibility is that nicotinic receptor modulator exerts its effect by acting at nicotinic acetylcholine receptors (nAChR), which are expressed in the striatum. There is a dense cholinergic innervation in striatum that closely coincides with dopaminergic neurons. Under physiological conditions, these cholinergic interneurons tonically release acetylcholine, which stimulates nicotinic receptors on dopaminergic nerve terminals to release dopamine. Similarly, exogenously applied agents such as nicotine result in a release of dopamine from striatal terminals.

### EXAMPLES

### Example 1

### Cloning and Expression of α-Synuclein

Recombinant protein expression and a purification system can be developed using standard techniques well known in the art. (See for example, Maniatis, T., Fritsch, E. F., Sambrook, J. Molecular Cloning: A Laboratory Manual, Second Edition (Cold Spring Harbor Laboratory, Cold Spring Harbor, N.Y., 1989).

Sample Preparation. In some embodiments, human wild type α-synuclein was expressed in the E. coli BL21(DE3) cell line transformed with pRK172/α-synuclein WT plasmid (kind gift of M. Goedert, MRC Cambridge) and was purified. 2 liters of cells were induced with 0.5 mM isopropyl β-D-thiogalactopyranoside and the resulting pellet was lysed by sonication at 0° C. in 50 mM NaCl, 20 mM Tris-HCl, 0.10% (v/v) Triton-X100, 0.20 mM phenylmethylsulfonyl fluoride at pH 8.0. The lysis suspension was brought to 30% saturation with ammonium sulfate at 0° C. (pellet discarded) followed by 50% saturation with ammonium sulfate. The resultant pellet was dialyzed against 50 mM NaCl, 20 mM Tris-HCl, pH 7.5, loaded onto a 25×130 mm DEAE Sepharose Fast Flow column (Amersham Pharmacia Biotech) equilibrated in the same buffer and eluted with a 50-450 mM NaCl gradient. Fractions containing α-synuclein were dialyzed exhaustively against water, clarified by centrifugation, and lyophilized for storage at −20 ° C. The resultant α-synuclein protein was judged to be >95% pure following SDS-polyacrylamide electrophoresis, gel-filtration and MS analysis. A stock solution of purified α-synuclein was briefly treated with 1 mM NaOH, neutralized and centrifuged for 30 min at 120,000 g before use to remove any aggregated material. Under appropriate conditions, recombinant α-synuclein formed the fibrils and aggregates as measured by primary and secondary assays, as described herein. In other embodiments mutant α-synuclein, or non-human α-synuclein is expressed and purified using similar methods.

In other embodiments, α-synuclein is amplified from a human brain cDNA library (Clontech) and upon confirmation of the sequence, polymerase-chain-reaction (PCR) is carried out. Primers 5′-CTCTCGGAGTGGCCATTCGA-′3 and 5′-GGCACATTGGAACTGAGCAC-3′ can amplify a fragment of the human α-synuclein cDNA. Amplification is performed by using AmpliTaq DNA Polymerase according to the manufacturer's protocol in a final volume of 100 ul; this is subjected to 35 cycles of PCR. The PCR product is purified by the Wizard PCR Preps DNA Purification System (Promega, Madison, Wis.) and is ligated into Srf I site of pCR-Script SK (+) vector according to the manufacturer's protocol (pCR-Script Amp SK (+) Cloning Kit, Stratagene Cloning Systems, La Jolla, Calif.). Following transformation of E. coli Epicurian Coli XL1-Blue MRF′ Kan supercompetent cells (Stratagene), separate colonies are selected, grown overnight, and plasmid DNA is purified by the Wizard Plus Minipreps DNA Purification System (Promega). The identity of the insert is confirmed by sequencing the plasmid DNA from individual colonies. The α-synuclein cDNA can be cloned into a pTYB 1 bacterial expression vector. Following transformation of ER2566 E. coli competent cells (New England BioLabs) and 6 h induction at 37° C. recombinant protein is isolated under denaturing conditions using Ni-NTA Resin. Under appropriate conditions, recombinant α-synuclein will form the predicted aggregates as measured by primary and secondary assays described herein.

### Example 2

### Primary Assays Utilized for Identifying Compounds that Inhibit, Reverse, Decrease, or Prevent α-Synuclein Fibrillation and/or Aggregation

### Primary Assay

Thioflavin Assay: The primary assay to detect α-synuclein fibrillation and/or aggregation utilized the fluorescent dye thioflavin T (referred to as ThT or TFT) which binds relatively specifically to fibrils and leads to enhanced fluorescence emission. Thioflavin T absorbs at 450 nm and emits at 485 nm; fluorescence increases 40-fold in the presence of beta-sheet conformation (LeVine and Scholten J. D. Screening for pharmacologic inhibitors of amyloid fibril formation. Meth Enzymol 1999, 309 (Ch. 29): 467-76.), Thioflavin T is obtained from Fluka Chemika or other vendors. Fibril formation was monitored by Thioflavin T fluorescence using a Fluoroskan Ascent CF plate reader (Labsystems, Inc.).

Aggregation Inhibition Thioflavin T Assay: In some embodiments an aggregation inhibition assay was conducted. Protein solutions of 150 μl containing 35 μM α-synuclein, 100 μM test compound and 10 μM Thioflavin T were incubated in 20 mM phosphate buffer, 100 mM NaCl, pH 7.0, in a 96-well plate, each well containing a 3 mm diameter Teflon spherical bead. The plate was incubated at 37° C. with shaking at 600 rpm, 2 mm diameter. The fluorescence was measured at 30 min intervals with excitation at 450 nm and emission at 485 nm. Multiple replicates were run.

In other embodiments, prior to assay, concentrated α-synuclein peptide 61-90 is diluted into assay buffer containing 20 uM Thioflavin T such that a signal to noise of approximately 5 to 1 is achieved. Test compounds are maintained in 8% DMSO at a concentration of 2mM. To break up super aggregates, the solution is sonicated using a Heat System sonicator with a microprobe. The samples are sonicated in 30 ml volume in a Coming 50 ml centrifuge tube for 25.3 second bursts. The assay is run in LJL HE (LJL Biosystems) plates as follows: 18 ul of alpha synuclein/thioflavin T mixture is added to the well followed by 2 ul of 30% DMSO or by the test compound diluted in 30% DMSO. As a control, buffer with Thioflavin T is added to separate wells in the absence of α-synuclein. The samples are incubated at room temperature at different time points and are then read on the LJL reader using wavelengths of 440 nm and 485 nm.

Disaggregation Thioflavin T Assay: In other embodiments, the α-synuclein is allowed to fully aggregate prior to adding the test compound and conducting a dissaggregation (or reversal of aggregation) assay. Degree of aggregation is measured by increase in Thioflavin T fluorescence. α-synuclein aggregation is conducted with or without seeding (using a peptide to enhance the rate of aggregation). The following peptides have been found to speed up synuclein aggregation: syn 61-90 (EQVTNVGGAVVTGVTAVAQKTVEGAGSIAA) and syn 61-75 (EQVTNVGGAVVTGVT). Twenty four micrograms (24 ug) of α-synuclein protein in assay buffer (0.2 M potassium chloride pH=6; 0.075% sodium azide) is added to microvolume multiwell plate holding 10 pL. Then, Thioflavin T is added to each well to a final concentration of 20 uM and contents are thoroughly mixed. α-synuclein is allowed to fully aggregate. The aggregated α-synuclein is assayed to screen compounds for antiaggregation properties. Each candidate compound is diluted in 30% DMSO (final concentration of 40 uM compound) and added to the well. The mixture containing the aggregated α-synuclein/Thioflavin T complex and the compound is incubated for 4 hours at room temperature. Compounds that promoted disaggregation of the complex are observed by a decrease of fluorescence compared to wells containing compounds not promoting disaggregation.

Because the test compounds could compete for the Thioflavin T site, quench the fluorescence of Thioflavin T, or other non-specific effects secondary assays using transmission electron microscopy and measurement of the soluble and insoluble protein were also used.

### Example 3

### Secondary Assays Utilized for Identifying Compounds that Inhibit, Reverse, Decrease, or Prevent α-Synuclein Fibrillation and/or Aggregation

Secondary Assays

Electron Microscopy: Those samples from Example 2 that showed inhibition were then analyzed by electron microscopy and assayed for soluble protein to confirm inhibition of fibrillation. Transmission electron micrographs were collected using a JEOL JEM-100B microscope, operating with an accelerating voltage of 80 kV. Samples were deposited on Formvar-coated 300 mesh copper grids and negatively stained with 1% aqueous uranyl acetate.

Centrifugation to separate soluble and insoluble protein: Samples after 72 hr incubation were centrifuged in an Eppendorf microfuge at 14,000 rpm for 30 min to separate soluble from insoluble material. The protein concentration in the uncentrifuged sample, and in the supernatant was analyzed for total protein concentration, usually using sodium dodecyl sulfate polyacrylamide gel electrophoresis (SDS-PAGE).

SDS-PAGE gel electrophoresis measurements. Samples for sodium dodecyl sulfate polyacrylamide gel electrophoresis (SDS-PAGE) were boiled in a water bath for 5 to 10 min in 4% SDS prior to electrophoretic separation. A PhastSystem with 8-25% gradient gels (Amersham Biosciences) was used and experiments were run with SDS buffer strips containing 0.20 M Tricine, 0.20 M Tris and 0.5% SDS, pH 7.5 for 30 min at 250 V. Gels were stained with Coomassie blue and quantified.

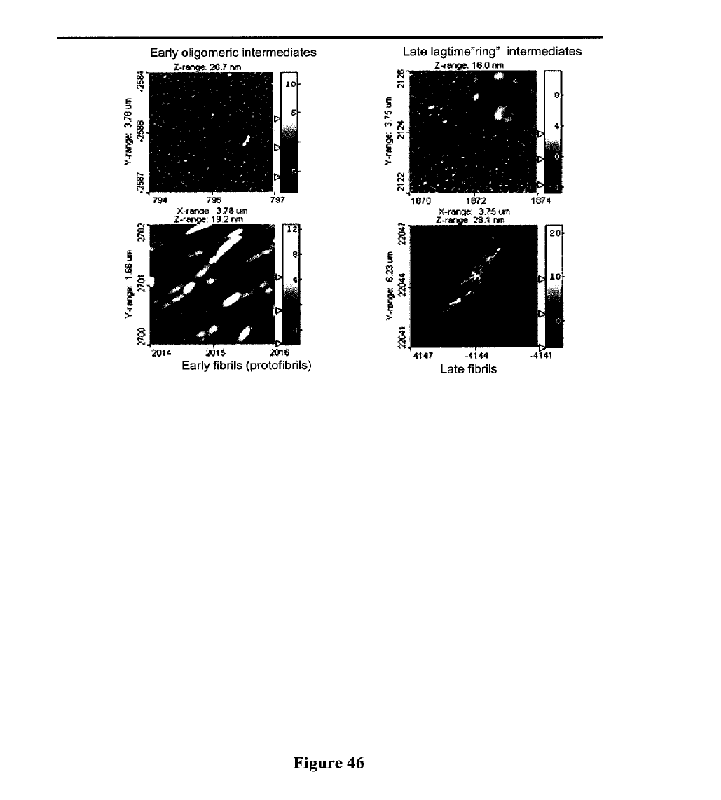

Atomic force microscopy (AFM): AFM images were collected with a PicoScan LE system (Molecular Imaging, Phoenix, AZ) equipped with Acoustic AC mode (Tapping mode) for ex-situ experiment. Triangular cantilevers with 160 kHz resonance frequency and 2 N/m spring constant, a V-shaped cantilever NSC16/AIBS (MikroMasch), were used in tapping mode imaging. The imaging was carried out at a scan rate of 1 line/s with 512 data points per line, at a drive current of 10±4 Å. Aliquots of 10 ul of sample were placed on a freshly cleaved mica substrate. After incubation for at least 60 min, the substrate was rinsed with water several times to remove salt and loosely bound protein and dried with high-purity nitrogen. Kinetics of α-synuclein fibrillation can be visualized with the use of this technique, as exemplified in FIG. 46.

### Example 4

### Compounds that Inhibit, Decrease, Reverse, or Prevent α-Synuclein Fibrillation and/or Aggregation

Compounds identified utilizing the methods described herein in Examples 1, 2 and 3 are described. Identified compounds either completely/ partially/significantly inhibit α-synuclein fibrillation, completely/partially/significantly reverse aggregation, display some/little/no Thioflavin T signal when tested in the Thioflavin T primary assay or completely/ partially/significantly display no fibrils when tested in the electron microscopy or atomic force microscopy secondary assays.

The NINDS Collection 2 Library was screened using the primary and secondary assay methods described herein. 5 compounds were complete inhibitors of fibrillation, 20 compounds were significant inhibitors of fibrillation and 9 compounds showed significant increases in the lagtime for fibrillation. Table 2 shows the results.

TABLE 2

| Compounds | Inhibition of α-synuclein fibrillation |
|---|---|
| pyrogallin | Complete inhibition of fibrillation: |
| cisplatin | No fibrils on EM |
| 1,4-naphthoquinone | |
| methyldopa | |
| gallate | |
| Cianidanol | Significant inhibition of fibrillation: |
| curcumin | Very low ThT signal |
| berberine chloride | Few fibrils on EM |
| daidzein | |
| isoproterenol hydrochloride | |
| quinalizarin | |
| merbromin | |
| norepinephrine | |
| benserazide hydrochloride | |
| hexachlorophene | |
| dopamine | |
| carbidopa | |
| dobutamine hydrochloride | |
| ethylnorepinephrine hydrochloride | |
| hydroquinone | |
| pyrvinium pamoate | |
| chlorophyllide Cu complex Na salt | |
| elaidyphosphocholine | |
| (antineoplastic) | |
| sulfasalazine | |
| tannic acid | |
| thyroid hormone | Significant increases in |
| purpurin | the lagtime for fibrillation |
| sodium beta-nicotinamide adenine | |
| dinucleotide phosphate | |
| lansoprazole | |
| dyclonine hydrochloride | |
| pramoxine hydrochloride | |
| azobenzene | |
| cefamandole sodium | |
| cephaloridine | |

The CHEMDIV Library was screened using the primary assay described above. The following compounds of formula II to VIII showed significant inhibition in the primary (Thioflavin) assay.

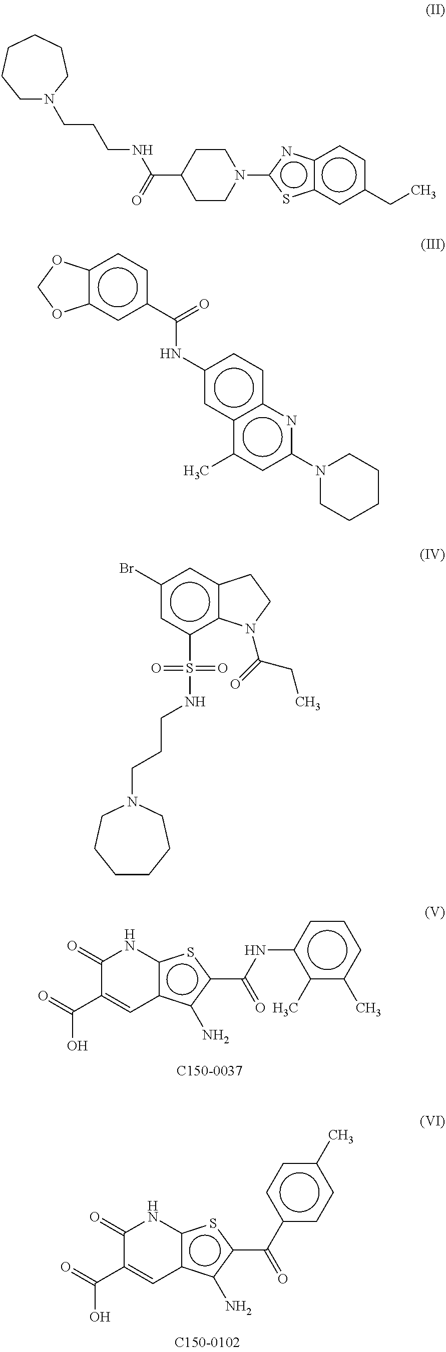

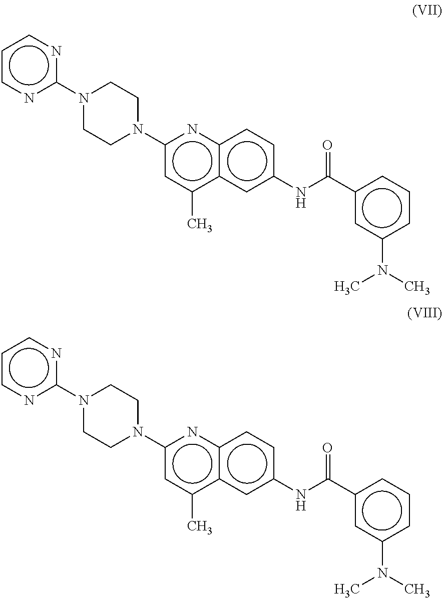

Flavonoids and related catechols were also screened. 40 of the compounds screened showed significant inhibition of fibrillation. Table 3 shows the results.

TABLE 3

| Compounds | Inhibition of α-synuclein fibrillation |
|---|---|
| myricetin | Significant inhibition: |
| 6,2′,3′-trihydroxyflavone | Low ThT signal |
| 5,7,3′,4′,5′-pentahydroxyflavone | Few fibrils on EM |
| 7,3′,4′,5′-tetrahydroxyflavone | |
| 6h (5,6,7,4′-tetrahydroxyflavone) | |
| baicalein | |
| eriodictyol | |
| 7,3′,4′-trihydroxyisoflavone | |
| epigallocatechin gallate | |
| quercetin | |
| gossypetin (3,5,7,8,3′,4′- | |
| hexahydroxyflavone) | |
| 2′,3′-dihydroxyflavone | |
| 3′,4′-dihydroxyflavone | |
| 5,6-dihydroxy-7-methoxyflavone | |
| baicalein-7-methyl ether | |
| l-dopa | |
| DOPAC | |
| homogentisic acid | |
| 6-hydroxydopamine | |
| epinephrine | |
| 3,4-dihydroxycinnamic acid | |
| 2,3-dihydroxynaphthalene | |
| 6-hydroxydopamine | |
| 3,4-dihydroxybenzoic acid | |
| 3,4,5-trihydroxybenzoic acid | |
| 1,2,3-trihydroxybenzoic acid | |
| 3,4-dihydroxycinnamic acid, | |
| gallate (gallic acid) | |
| benzoquinone | |
| hydroquinone | |
| catechol | |
| rifampicin | |
| rosmarinic acid | |
| baicalin | |
| tanshinones I and II | |
| emodin | |
| procyanidin b4 | |
| resveratrol | |
| rutin | |
| fisetin | |
| luteolin | |
| fustin | |
| epicatechin gallate (ecgc) | |
| catechin | |
| alizarin | |

Most significant polyphenol inhibitors were also found to be disaggregators. Among the best disaggregators were the following: baicalein; hydroxyapigenim; myricetin; 5,7,3′,4′,5′-Pentahydroxyflavone; 7,3′,4′,5′-tetrahydroxyflavone; 6,2′,3′-trihydroxyflavone; 7,3′,4′-trihydroxyisoflavone; epigallocatechin gallate; gossypetin (3,5,7,8,3′,4′-Hexahydroxyflavone); 3′,4′-dihydroxyflavone; fisetin; luteolin; pyrogallol; 2′,3′-dihydroxyflavone; and 3′,4′-dihydroxyflavonol.

The Spectrum Collection from MicroSource Discovery was screened using the methods described herein. The results are in Table 4.

TABLE 4

| Compounds | Inhibition of α-synuclein fibrillation |
|---|---|
| baicalein | Complete Inhibitors: |
| tannic acid | No change in ThT fluorescence |
| eriodyctol | at 72 hours |
| rosmarinic acid | |
| carboplatin | |
| cisplatin | |
| purpurogallin-4-carboxylic acid | |
| koparin | |
| 2,3,4-trihydroxy-4′- | |
| ethexybenzophenone | |
| baeomycesic acid | |
| hamtoxylin | |
| iriginol hexaaceatate | |
| 4-acetoxyphenol | |
| theaflavin monogallate | |
| theaflavin digallate | |
| stictic acid | |
| purpurogallin | |
| pyrogallin | |
| 2,5-dihydroxy-3,4-dimethoxy-4′- | |
| ethoxybenzophenone | |
| benserazide hydrochloride | Partial Inhibitors: |
| promethazine hydrochloride | Longer lag time for fibrillation |
| oxidopamine hydrochloride | Lower ThT fluorescence |
| pyrantel pamoate | |
| elaidylphosphocholine | |
| amphotericin B | |
| gallic acid | |
| fumarprotocetraric acid | |
| theaflavin | |
| haematoxylin pentaacetate | |
| 4-methoxydalbergione | |
| epigallocatechin-3-monogallate | |
| isoproterenol hydrochloride | |
| rolitetracycline | |
| 7,3′-dimethoxyflavone | |
| liquiritigenin dimethyl ether | |
| catechin pentaacetate | |
| apigenin | |
| 3,4-dedesmethyl-5-deshydroxy-3′- | |
| ethoxyscleroin | |

Table 5 displays compounds collectively identified from using the methods described herein. These compounds modulate, inhibit, reverse, decrease or prevent α-synuclein fibrillation and/or aggregation as identified by the primary and secondary assay described herein. The group of compounds presented in Table 5 is provided for the ease of viewing. Each compound named is an individual compound. In certain embodiments compounds can include derivatives, analogs, or metabolites thereof.

TABLE 5
Compounds that modulate, inhibit, reverse, decrease or prevent α-synuclein fibrillation/and or
aggregation

| | | |
|---|---|---|
| apomorphine | cefamandole sodium | fisetin |
| pyrogallol | cephaloridine | luteolin |
| 1,4-naphthoquinone | myricetin | fustin |
| cisplatin | 6,2′,3′-trihydroxyflavone | epicatechin gallate |
| isoproterenol | 5,7,3′,4′,5′-pentahydroxyflavone | catechin |
| pyrogallin | 7,3′,4′,5′-tetrahydroxyflavone | alizarin |
| cianidanol | (5,6,7,4′-tetrahydroxyflavone) | tannic acid |
| sulfasalazine | baicalein | eriodyctol |
| quinalizarin | eriodictyol | carboplatin |
| benserazide | 7,3′,4′-trihydroxyisoflavone | purpurogallin-4-carboxylic acid |
| hexachlorophene | epigallocatechin gallate | koparin |
| pyrvinium pamoate | quercetin | 2,3,4-trihydroxy-4′-ethexybenzophenone |
| dobutamine | gossypetin (3,5,7,8,3′,4′- | baeomycesic acid |
| | hexahydroxyflavone) | |
| methyl-dopa | 2′,3′-dihydroxyflavone | hamtoxylin |
| curcumin | 3′,4′-dihydroxyflavone | iriginol hexaaceatate |
| berberine chloride | 5,6-dihydroxy-7- | 4-acetoxyphenol |
| | methoxyflavone | |
| daidzein | baicalein-7-methyl ether | theaflavin monogallate |
| merbromin | Levodopa (L-Dopa) | theaflavin digallate |
| norepinephrine | DOPAC | stictic acid |
| dopamine hydrochloride | homogentisic acid | purpurogallin |
| carbidopa | 6-hydroxydopamine | 2,5-dihydroxy-3,4-dimethoxy-4′- |
| | | ethoxybenzophenone |
| ethylnorepinephrine hydrochloride | epinephrine | promethazine hydrochloride |
| tannic acid | 3,4-dihydroxycinnamic acid | oxidopamine hydrochloride |
| elaidyphosphocholine | 2,3-dihydroxynaphthalene | pyrantel pamoate |
| hydroquinone | 3,4-dihydroxybenzoic acid | elaidylphosphocholine |
| chlorophyllide Cu complex Na salt | 3,4,5-trihydroxybenzoic acid | amphotericin B |
| methyldopa | 1,2,3-trihydroxybenzoic acid | gallic acid |
| isoproterenol hydrochloride | gallate (gallic acid) | fumarprotocetraric acid |
| benserazide hydrochloride | benzoquinone | theaflavin |
| dopamine | catechol | haematoxylin pentaacetate |
| dobutamine hydrochloride | rifampicin | 4-methoxydalbergione |
| thyroid hormone | rosmarinic acid | epigallocatechin-3-monogallate |
| purpurin | baicalin | rolitetracycline |
| sodium beta-nicotinamide adenine | tanshinones I and II | 7,3′-dimethoxyflavone |
| dinucleotide phosphate | | |
| lansoprazole | emodin | liquiritigenin dimethyl ether |
| dyclonine hydrochloride | procyanidin B4 | catechin pentaacetate |
| pramoxine hydrochloride | resveratrol | apigenin |
| azobenzene | rutin | 3,4-dedesmethyl-5-deshydroxy-3′- |
| | | ethoxyscleroin |

Of the compounds identified in Table 5, apomorphine, L-Dopa, norepinephrine, and benserazide are in clinical use or consideration for use for the treatment of the primary symptoms associated with Parkinson's disease.

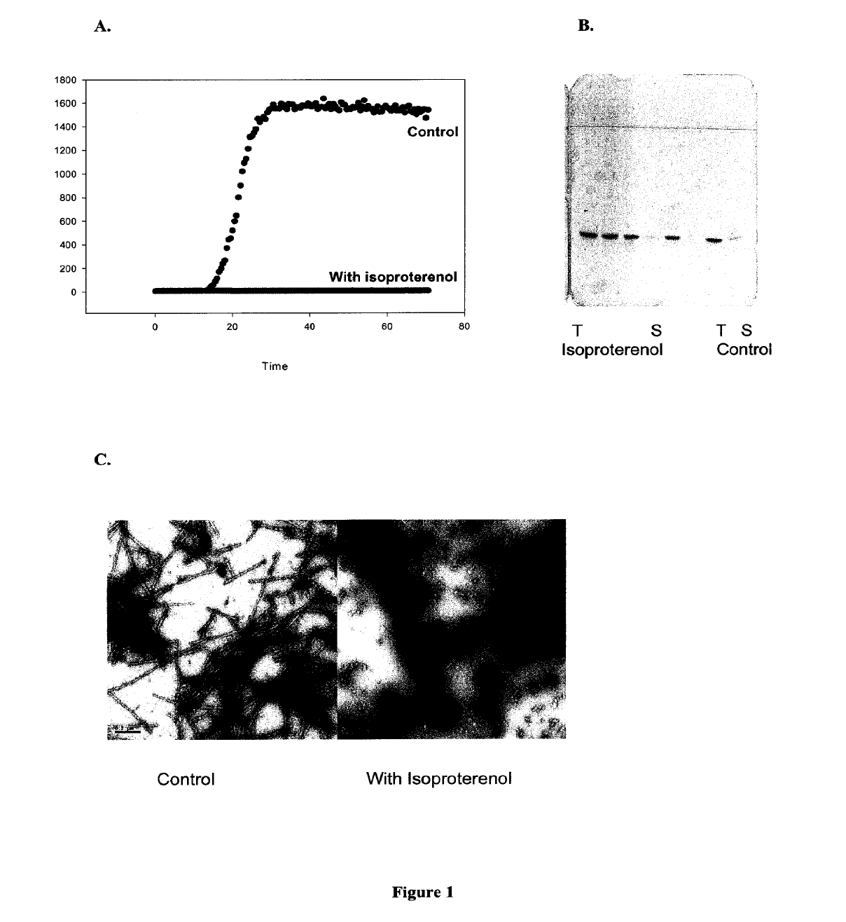

FIGS. 1-46 show representative data for inhibitors of α-synuclein fibrillation and/or aggregation.

FIG. 1A shows inhibition of α-synuclein fibril formation by isoproterenol monitored by the increase in Thioflavin T fluorescence on binding to fibrils. Isoproterenol is a bronchodilator currently used to prevent and treat wheezing, shortness of breath, and troubled breathing caused by asthma, chronic bronchitis, emphysema, and other lung diseases. It relaxes and opens air passages in the lungs, making it easier to breathe. FIG. 1 shows a very low Thioflavin T signal in samples treated with isoproterenol when compared with control samples. FIG. 1B shows the concentration of protein in the supernatant of isoproterenol and control samples. After centrifugation, >90% of the protein is in the supernatant of samples treated with the isoproterenol whereas <15% of the protein is in the supernatant of control samples. FIG. 1C depicts an EM picture of fibril formation in isoproterenol and control samples. Few fibrils are seen in the sample treated with isoproterenol.

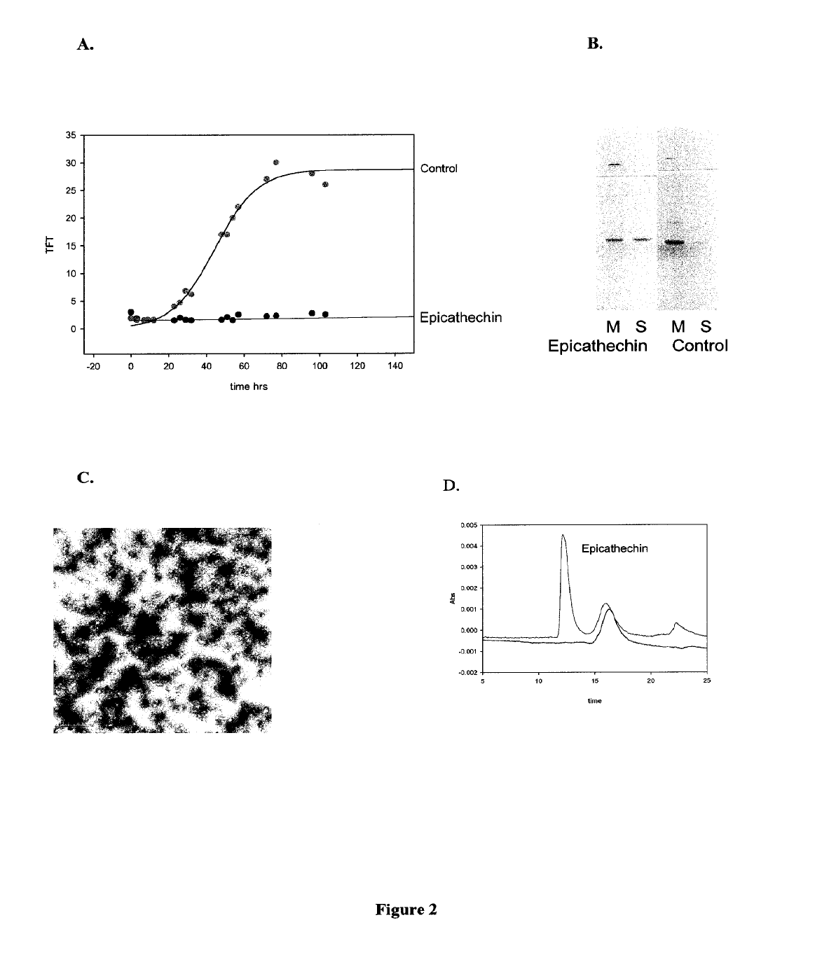

FIG. 2 shows results from experiments with epicatechin. FIG. 2A shows a very low Thioflavin T signal in samples treated with epicatechin when compared with control samples. FIG. 2B shows SDS PAGE of supernatant (S) vs. total mixture (M). This shows that most protein still soluble after incubation with Epicatechin. FIG. 2C confirms by EM negligible fibril formation when epicatechin is used. SEC HPLC shows oligomer formation with Epicatechin (red), none with control (black) (FIG. 2D).

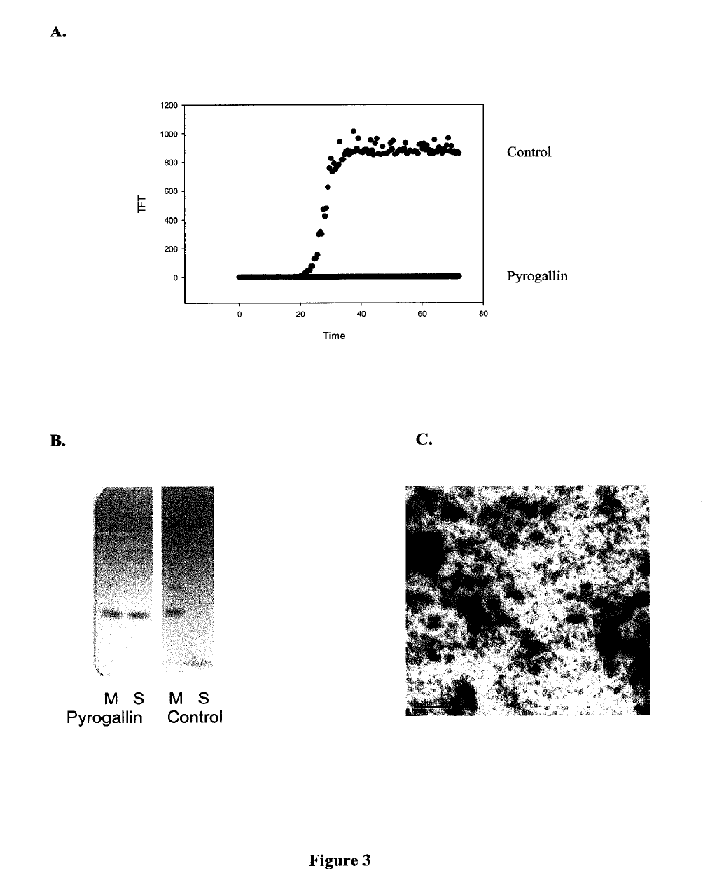

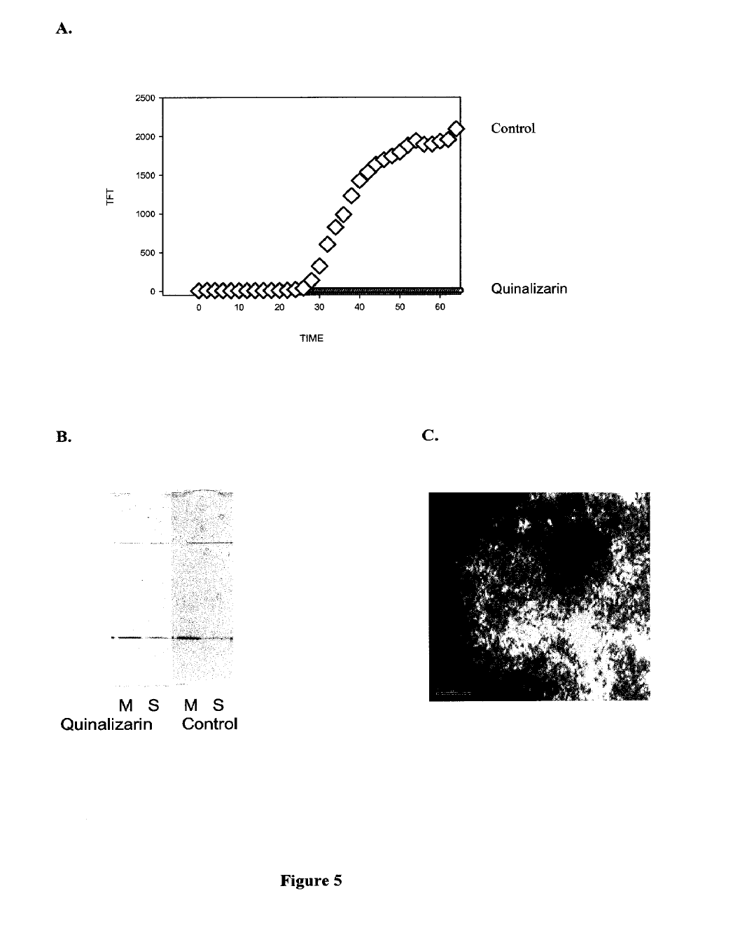

FIG. 3 shows results from experiments with pyrogallin. FIG. 3A shows negligible fibril formation by Thioflavin T fluorescence in samples treated with pyrogallin when compared with control samples. FIG. 5B shows SDS PAGE of supernatant (S) vs. total mixture (M). This shows that most protein still soluble after incubation with pyrogallin. FIG. 3C confirms by EM negligible fibril formation when pyrogallin is used.

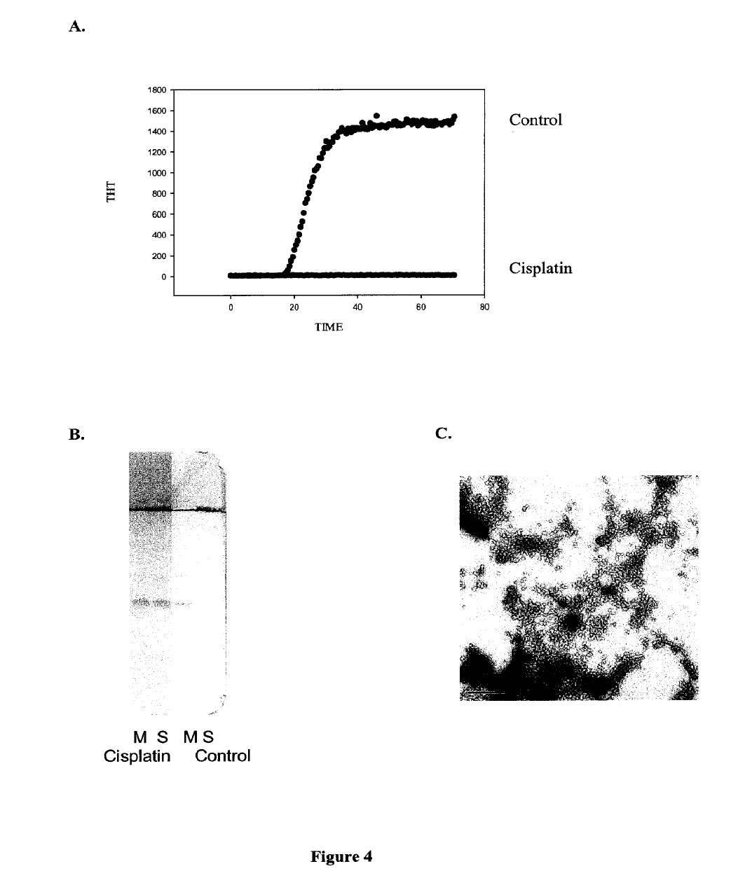

FIG. 4 shows results from experiments with cisplatin. FIG. 4A shows negligible fibril formation by Thioflavin T fluorescence in samples treated with cisplatin when compared with control samples . FIG. 4B shows SDS PAGE of supernatant (S) vs. total mixture (M). This shows that most protein still soluble after incubation with cisplatin. FIG. 4C confirms by EM negligible fibril formation when cisplatin is used.

FIG. 5 shows results from experiments with quinalizarin. FIG. 5A shows negligible fibril formation by Thioflavin T fluorescence in samples treated with quinalizarin when compared with control samples (white squares). FIG. 5B shows SDS PAGE of supernatant (S) vs. total mixture (M). This shows that most protein still soluble after incubation with quinalizarin. FIG. 5C confirms by EM negligible fibril formation when quinalizarin is used.

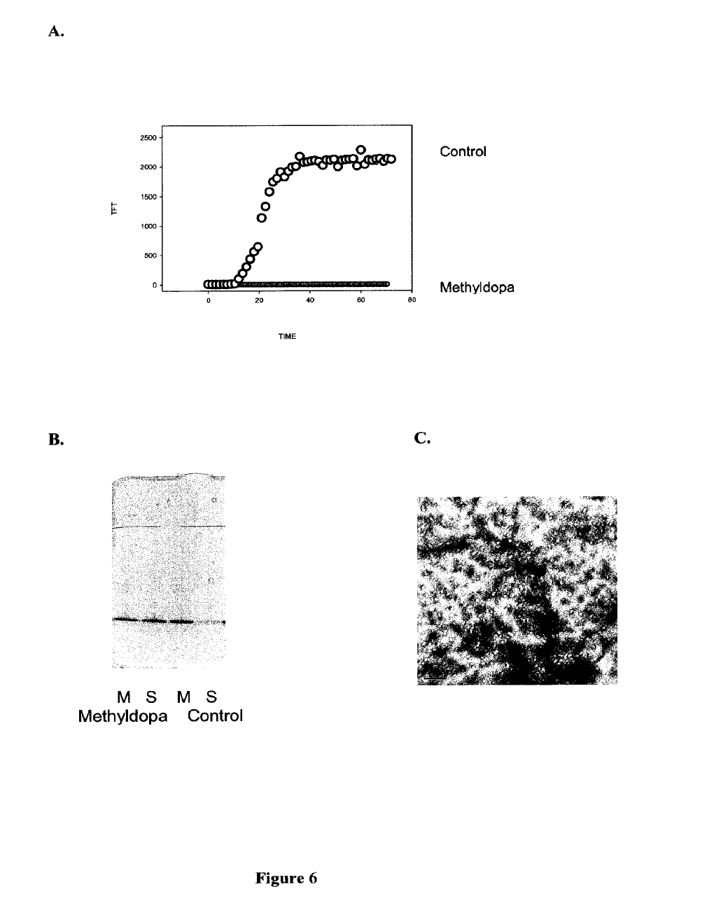

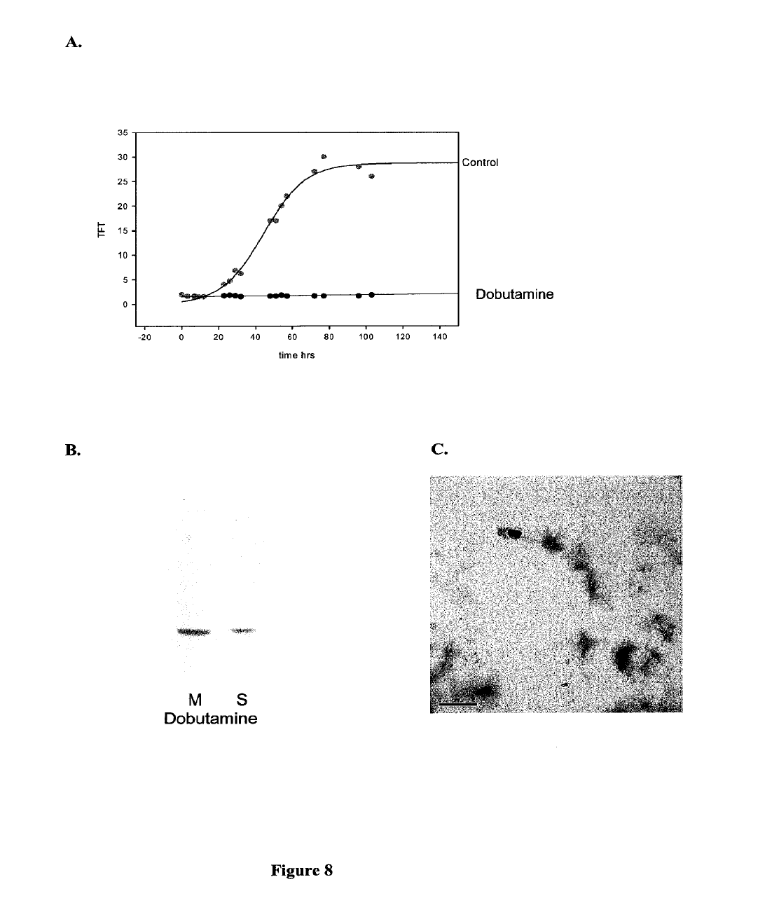

FIG. 6 shows results from experiments with Methyldopa. FIG. 6A shows negligible fibril formation by Thioflavin T fluorescence in samples treated with Methyldopa when compared with control samples. FIG. 8B shows SDS PAGE of supernatant (S) vs. total mixture (M). This shows that most protein still soluble after incubation with Methyldopa. FIG. 6C confirms by EM negligible fibril formation when Methyldopa is used.

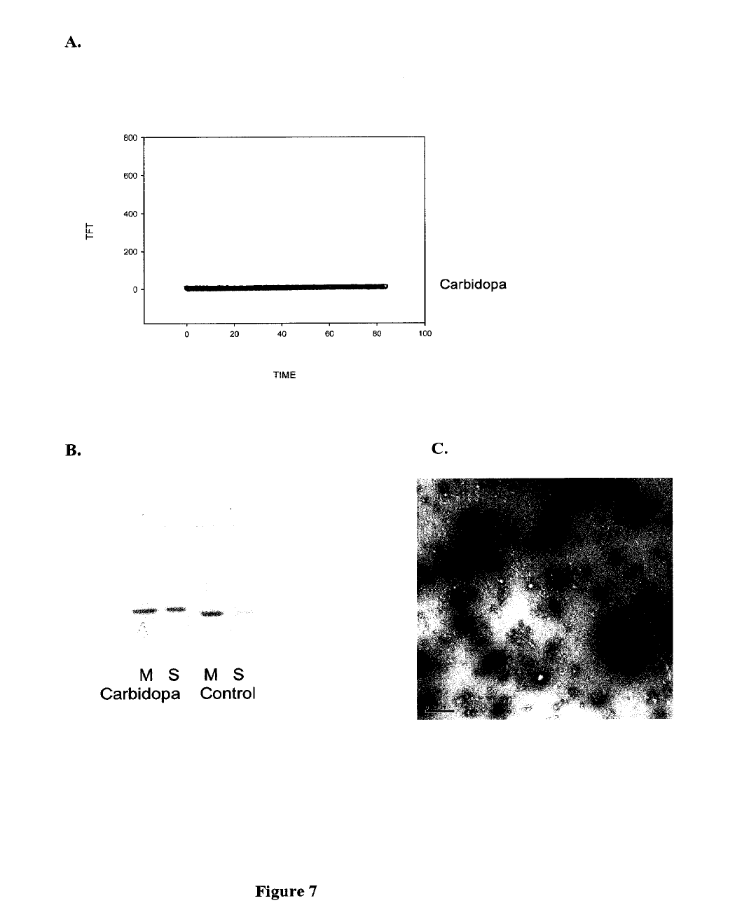

FIG. 7 shows results from experiments with carbidopa. FIG. 7A shows negligible fibril formation by Thioflavin T fluorescence in samples treated with carbidopa. FIG. 7B shows SDS PAGE of supernatant (S) vs. total mixture (M). This shows that most protein still soluble after incubation with carbidopa. FIG. 7C confirms by EM negligible fibril formation when carbidopa is used.

FIG. 8 shows results from experiment with dobutamine. FIG. 8A shows negligible fibril formation by Thioflavin T fluorescence in samples treated with dobutamine when compared with control samples. FIG. 8B shows SDS PAGE of supernatant (S) vs. total mixture (M). This shows that most protein still soluble after incubation with dobutamine. FIG. 8C confirms by EM negligible fibril formation when dobutamine is used.

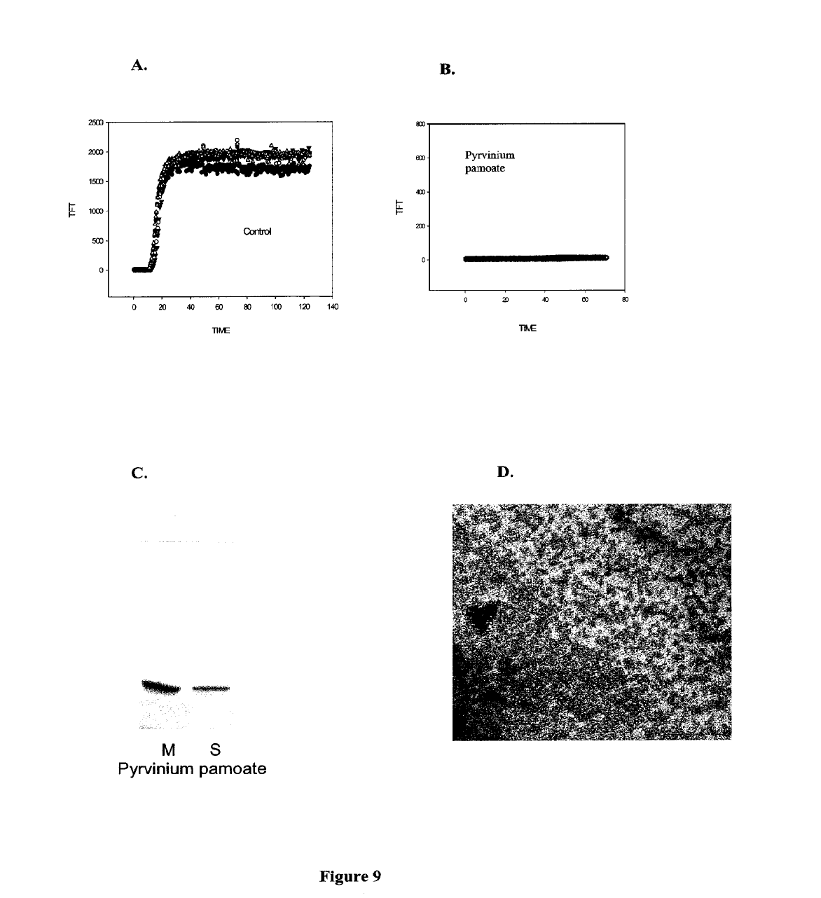

FIG. 9 shows results from experiments with pyrvinium pamoate. FIGS. 9A and B show negligible fibril formation by Thioflavin T fluorescence in samples treated with pyrvinium pamoate (B) when compared with control samples (A). FIG. 9C shows SDS PAGE of supernatant vs. total mixture. This shows that most protein still soluble after incubation with pyrvinium pamoate. FIG. 9D confirms by EM negligible fibril formation when pyrvinium pamoate is used.

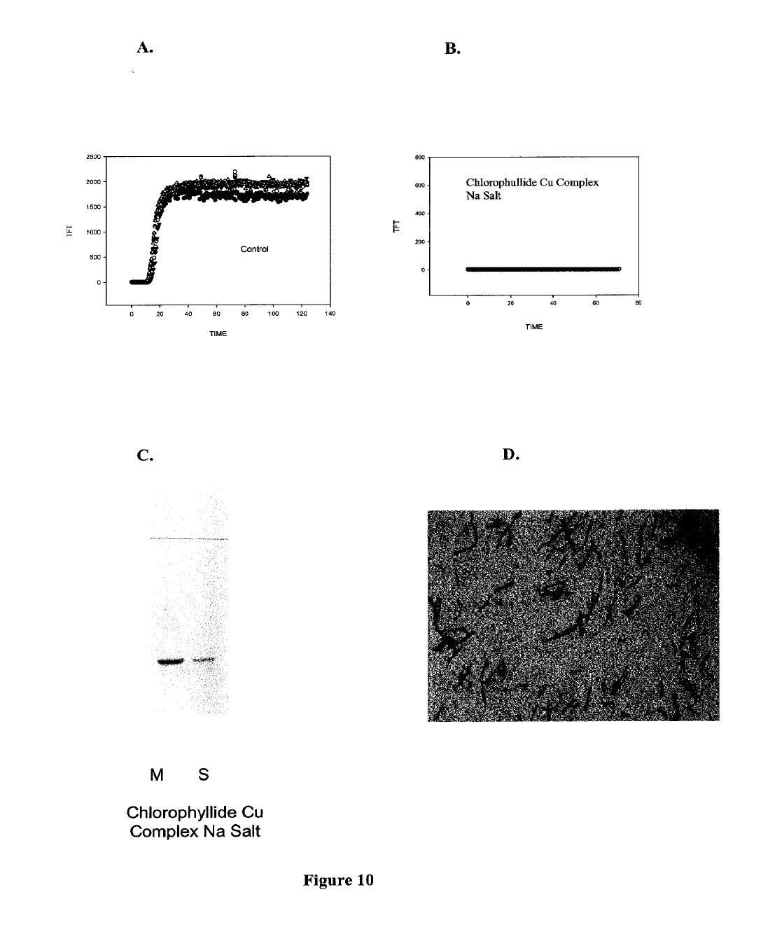

FIG. 10 shows results from experiments with chlorophyllide Cu complex Na salt. FIGS. 10A and B show negligible fibril formation by Thioflavin T fluorescence in samples treated with chlorophyllide Cu complex Na salt (B) when compared with control samples (A). FIG. 10C shows SDS PAGE of supernatant (S) vs. total mixture (M). This shows that most protein still soluble after incubation. FIG. 10D confirms by EM negligible fibril formation when chlorophyllide Cu complex Na salt is used.

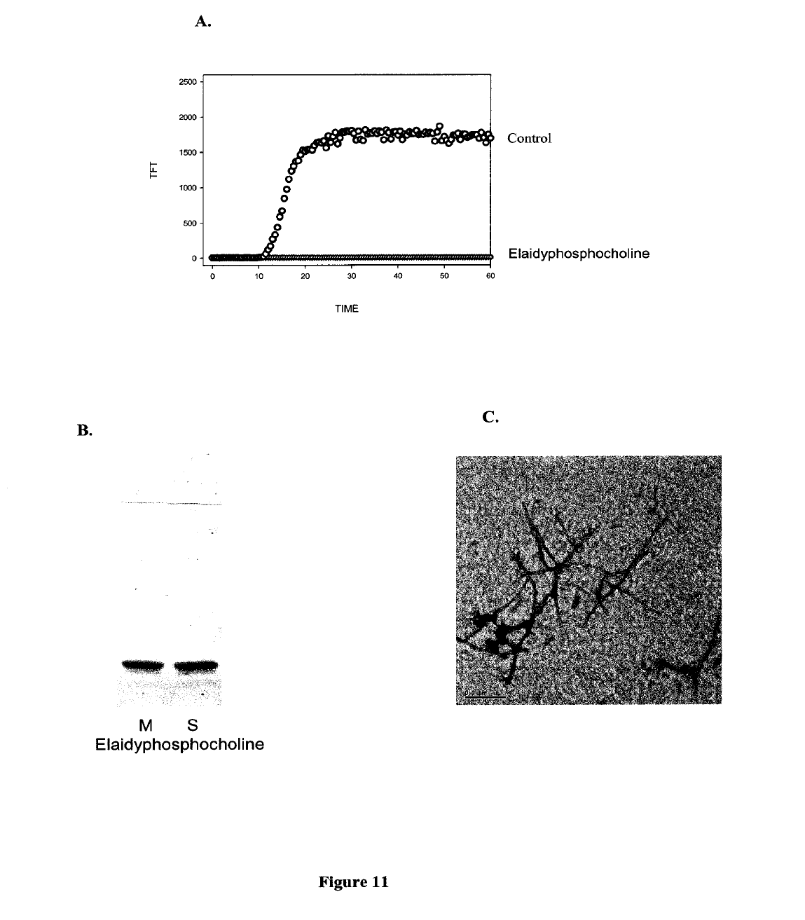

FIG. 11 shows results from experiments with elaidylphosphocholine. FIG. 11A shows negligible fibril formation by Thioflavin T fluorescence is samples treated with elaidylphosphocholine when compared with control samples (white circles). FIG. 11B shows SDS PAGE of supernatant (S) vs. total mixture (M). This shows that most protein still soluble after incubation with elaidylphosphocholine. FIG. 11C confirms by EM negligible fibril formation when elaidylphosphocholine is used.

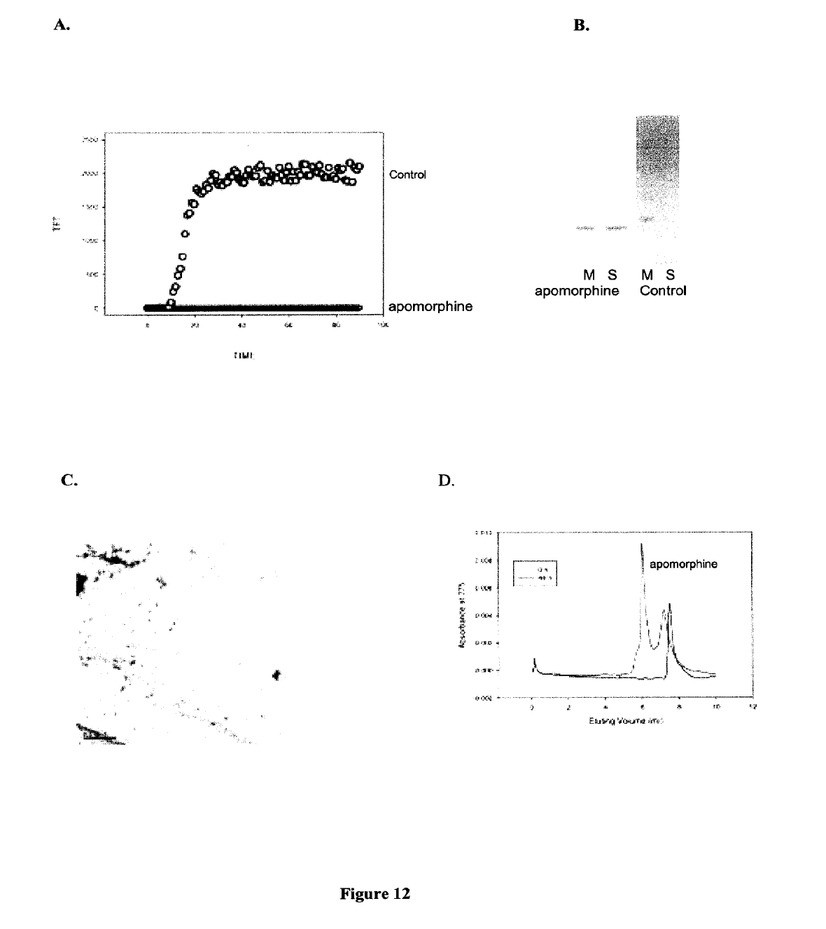

FIG. 12 shows results from experiments with apomorphine. FIG. 12A shows a very low Thioflavin T signal in samples treated with apomorphine when compared with control samples. FIG. 12B shows SDS PAGE of supernatant (S) vs. total mixture (M). This shows that most protein still soluble after incubation with apomorphine. FIG. 12C confirms by EM negligible fibril formation when apomorphine is used. SEC HPLC shows oligomer formation with apomorphine and none with control (FIG. 12D).

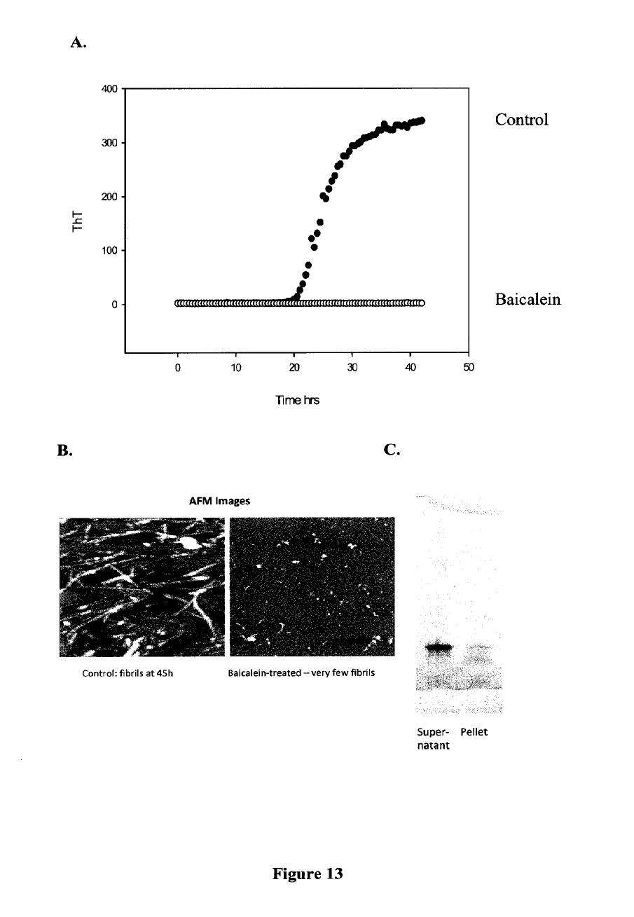

FIG. 13 shows results from experiments with baicalein. FIG. 13A shows a very low Thioflavin T signal in samples treated with baicalein when compared with control samples. FIG. 13B shows very few fibrils with atomic force microscopy at 45 hours post baicalein treatment, compared to control. FIG. 13C shows SDS PAGE of supernatant versus pellet. This shows that most protein still soluble after incubation with baicalein.

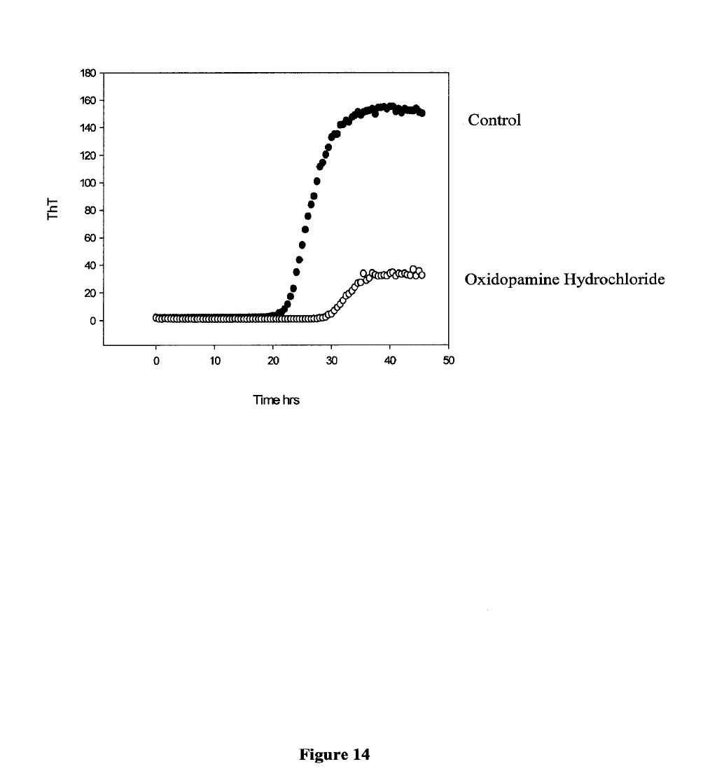

FIG. 14 shows Thioflavin T assay results from experiments with oxidopamine hydrochloride.

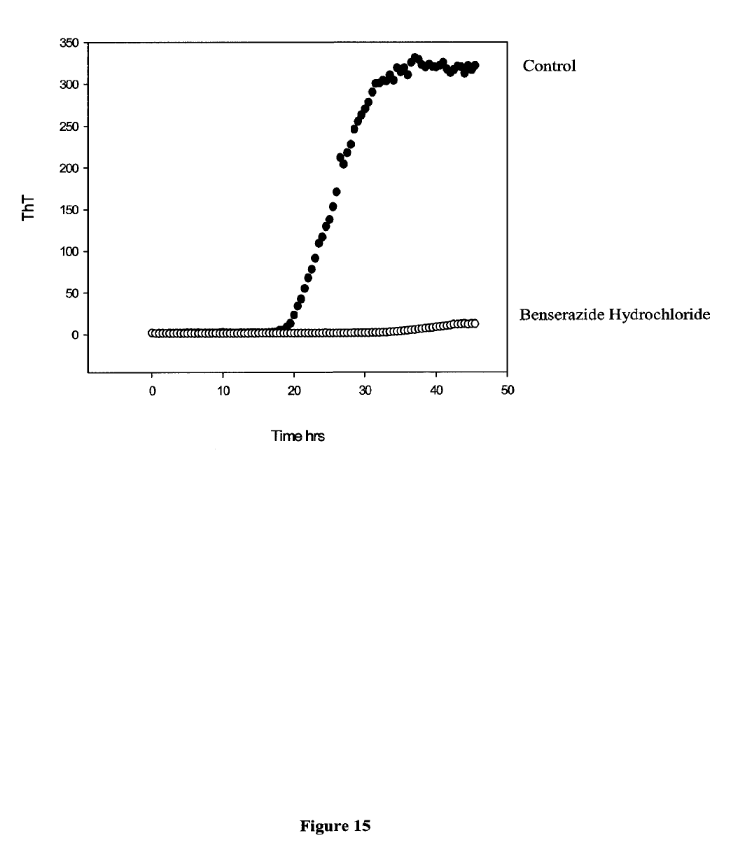

FIG. 15 shows Thioflavin T assay results from experiments with benserazide hydrochloride.

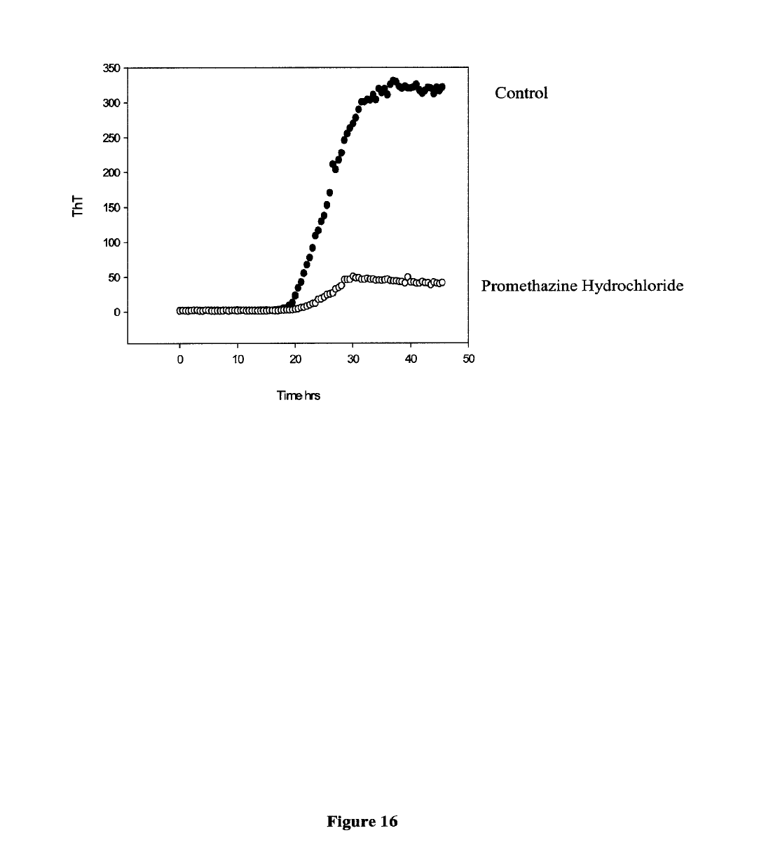

FIG. 16 shows Thioflavin T assay results from experiments with promethazine hydrochloride

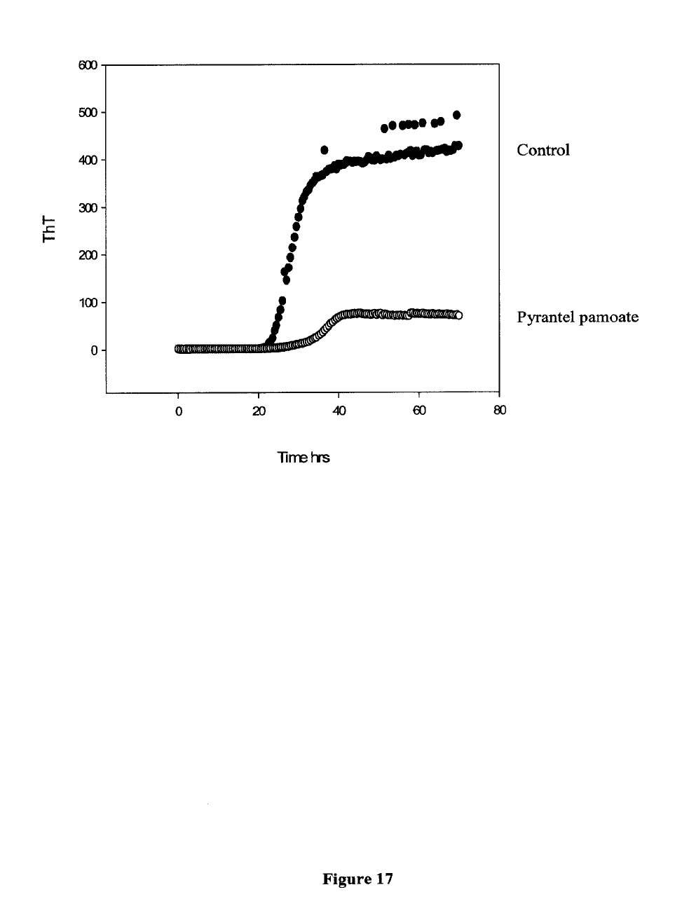

FIG. 17 shows Thioflavin T assay results from experiments with pyrantel pamoate.

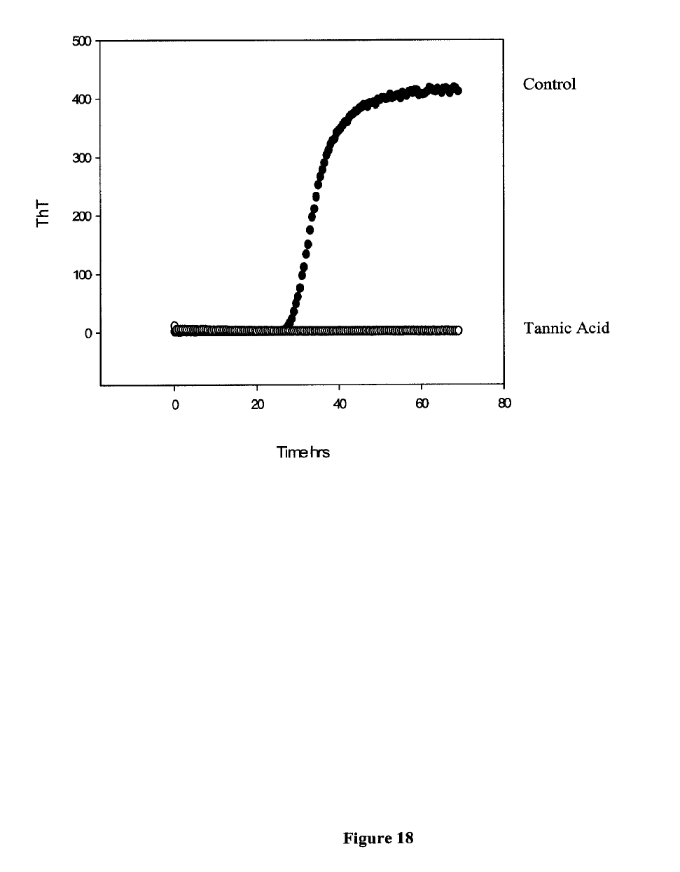

FIG. 18 shows Thioflavin T assay results from experiments with tannic acid.

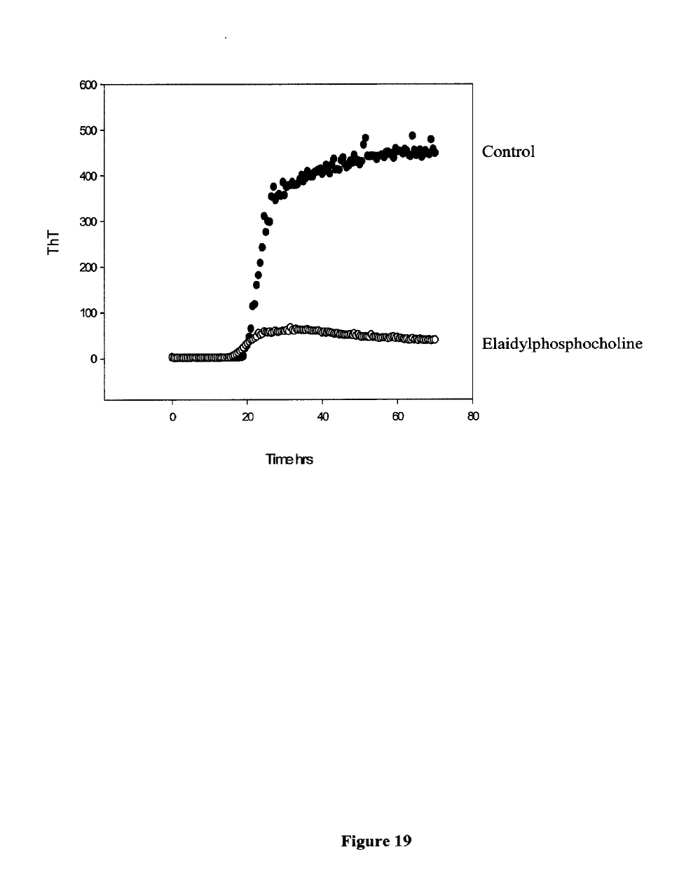

FIG. 19 shows Thioflavin T assay results from experiments with elaidylphosphocholine.

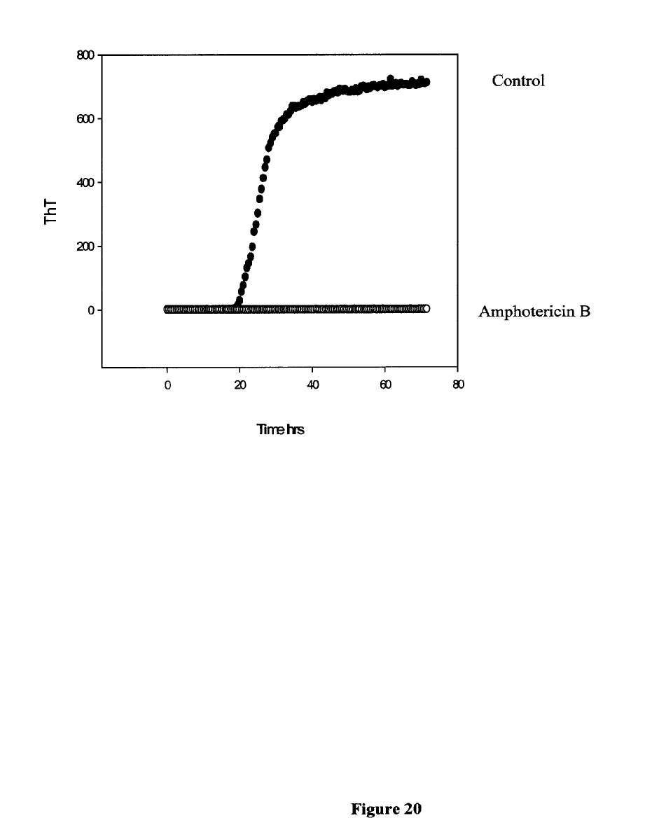

FIG. 20 shows Thioflavin T assay results from experiments with amphotericin B.

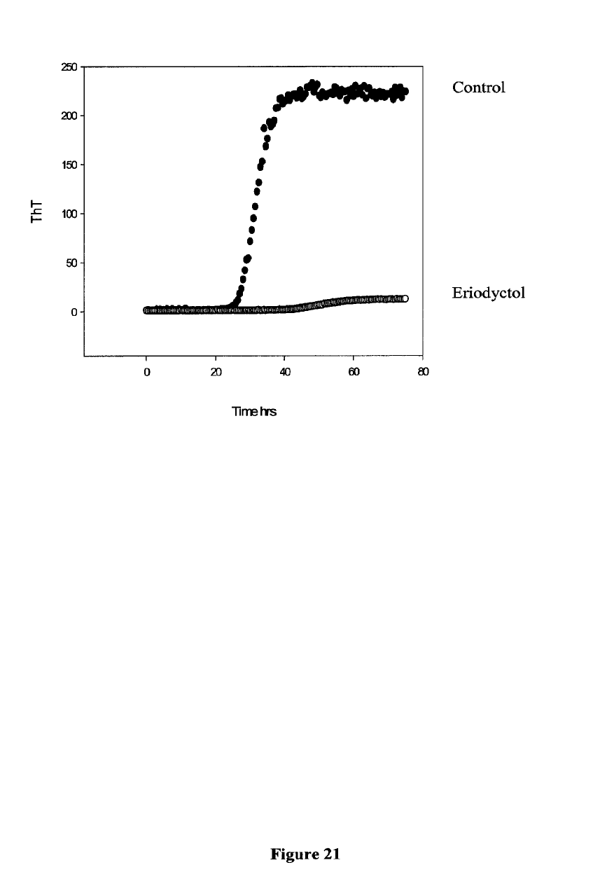

FIG. 21 shows Thioflavin T assay results from experiments with eriodyctol.

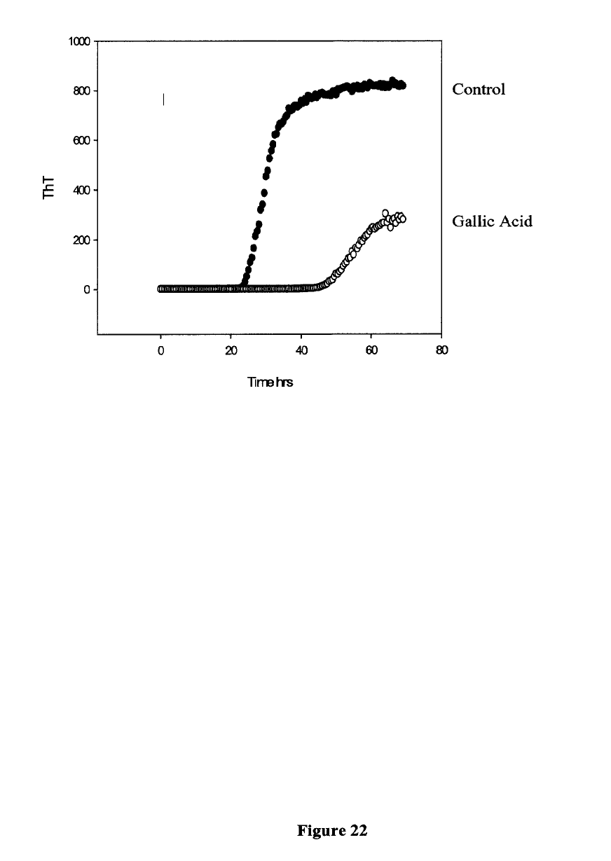

FIG. 22 shows Thioflavin T assay results from experiments with gallic Acid.

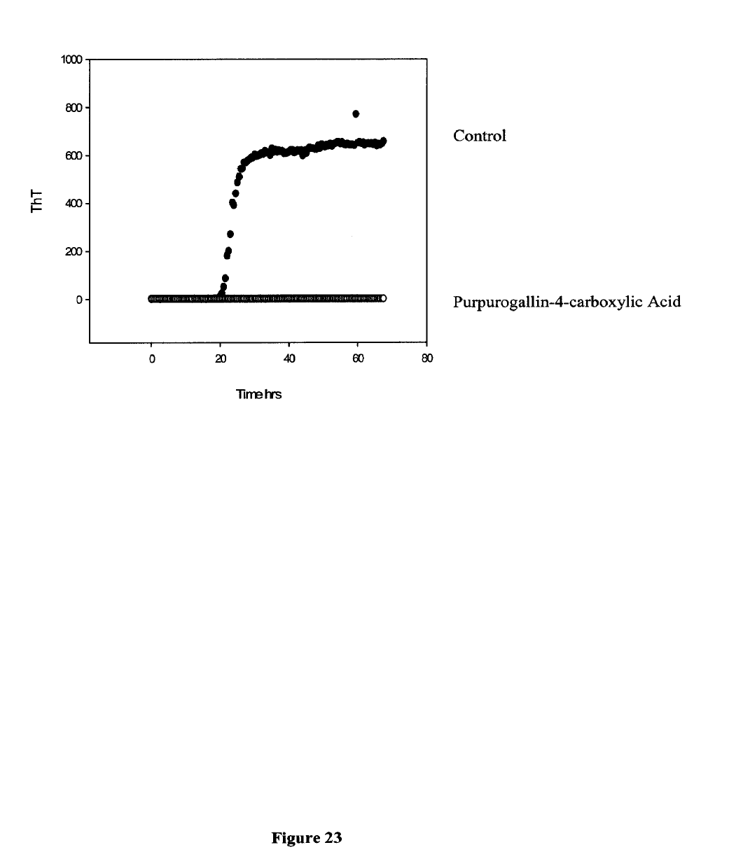

FIG. 23 shows Thioflavin T assay results from experiments with purpurogallin-4-carboxylic Acid.

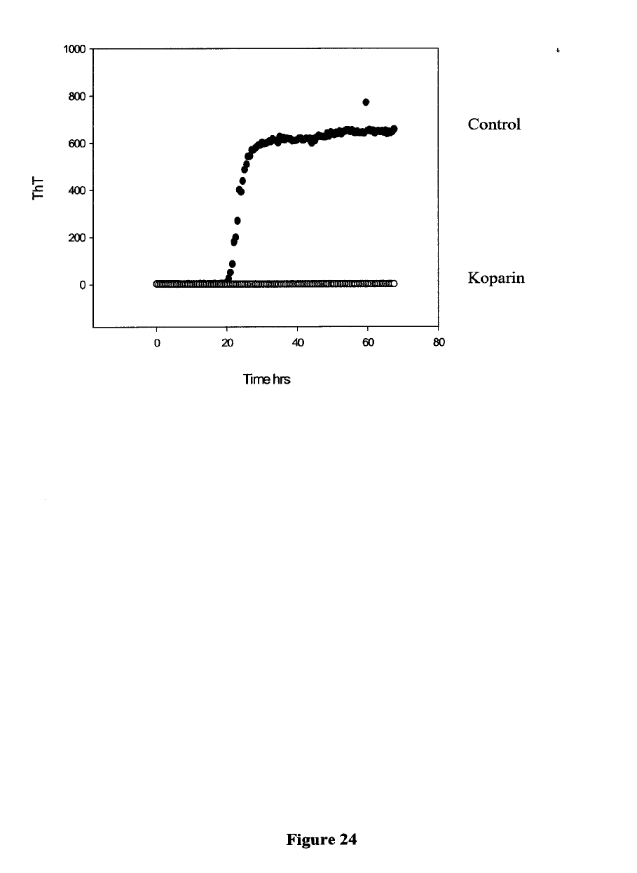

FIG. 24 shows Thioflavin T assay results from experiments with Koparin.

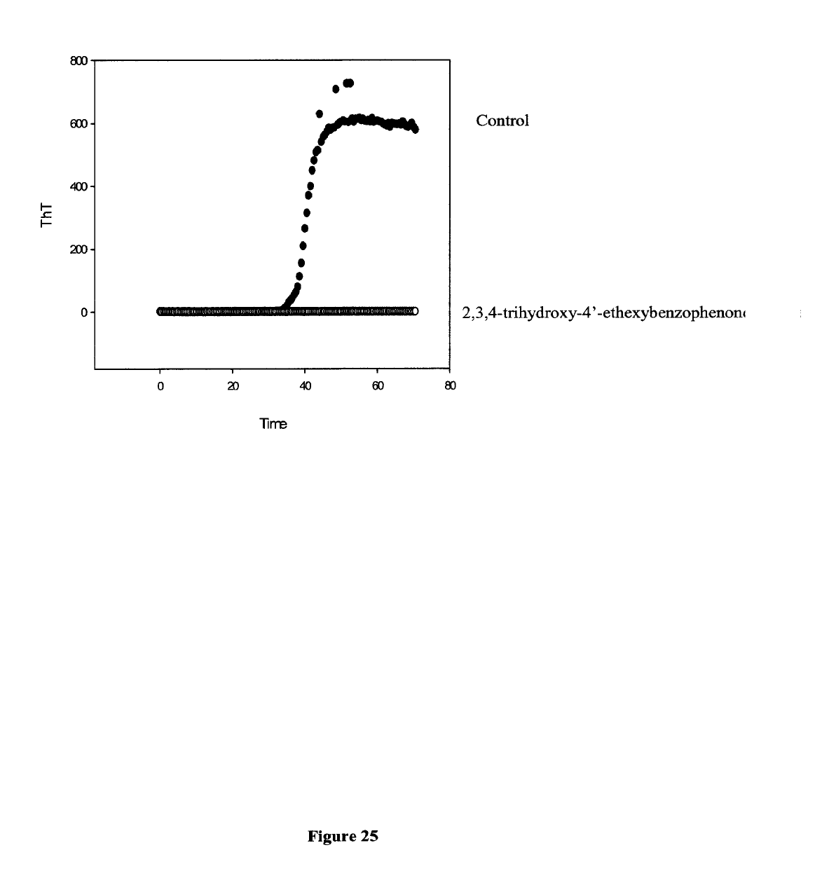

FIG. 25 shows Thioflavin T assay results from experiments with 2,3,4-trihydroxy-4′-ethexybenzophenone.

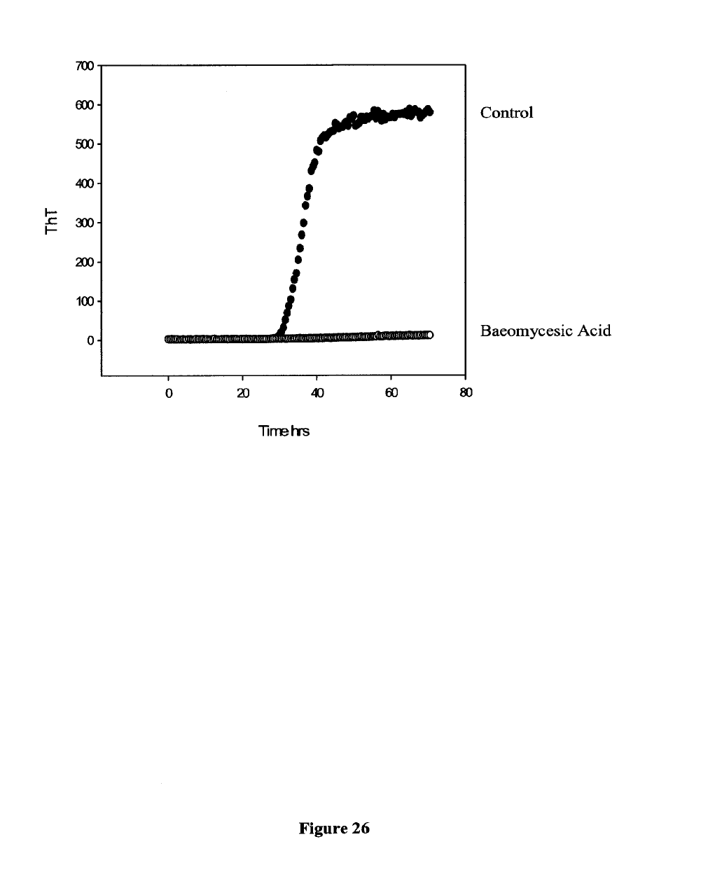

FIG. 26 shows Thioflavin T assay results from experiments with baeomycesic Acid.

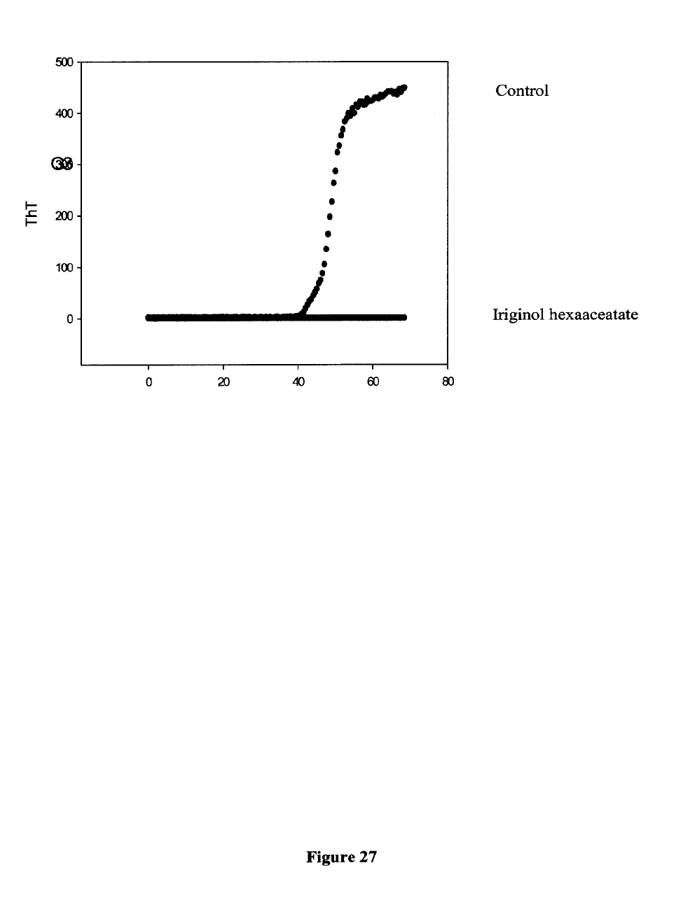

FIG. 27 shows Thioflavin T assay results from experiments with iriginol hexaaceatate.

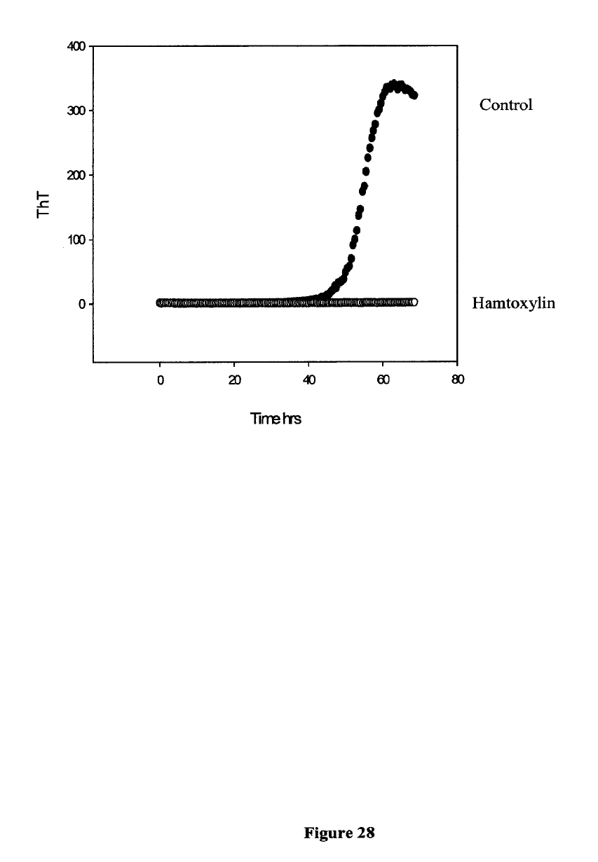

FIG. 28 shows Thioflavin T assay results from experiments with hamtoxylin.

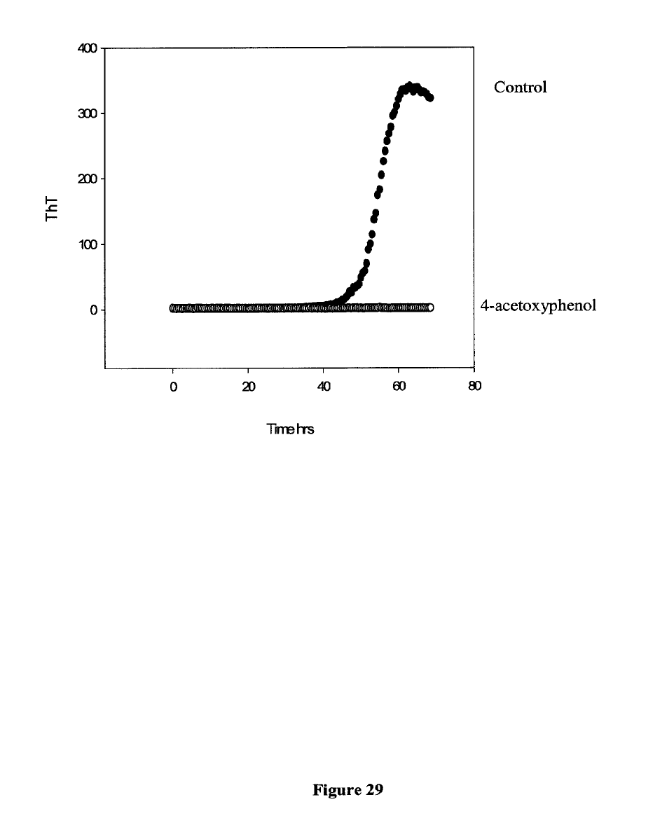

FIG. 29 shows Thioflavin T assay results from experiments with 4-acetoxyphenol.

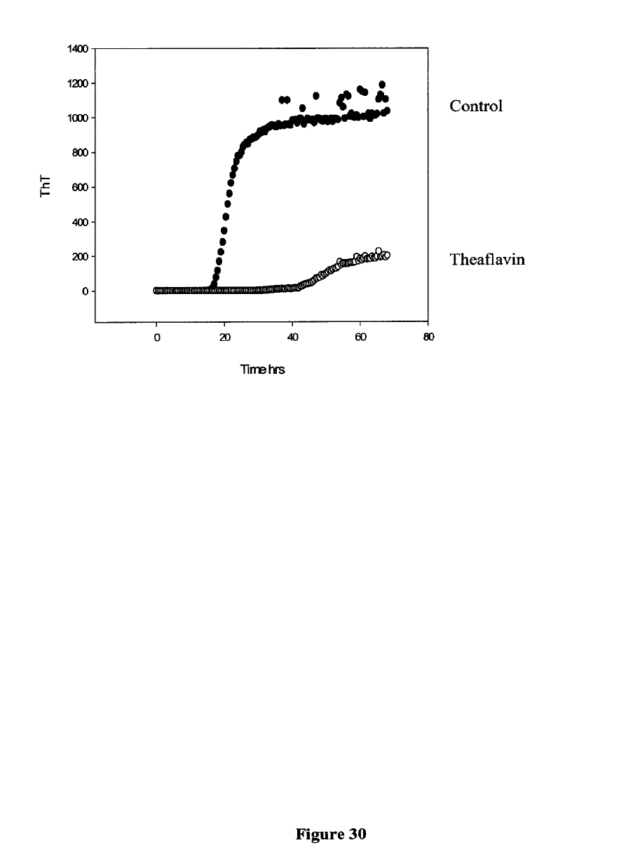

FIG. 30 shows Thioflavin T assay results from experiments with theaflavin.

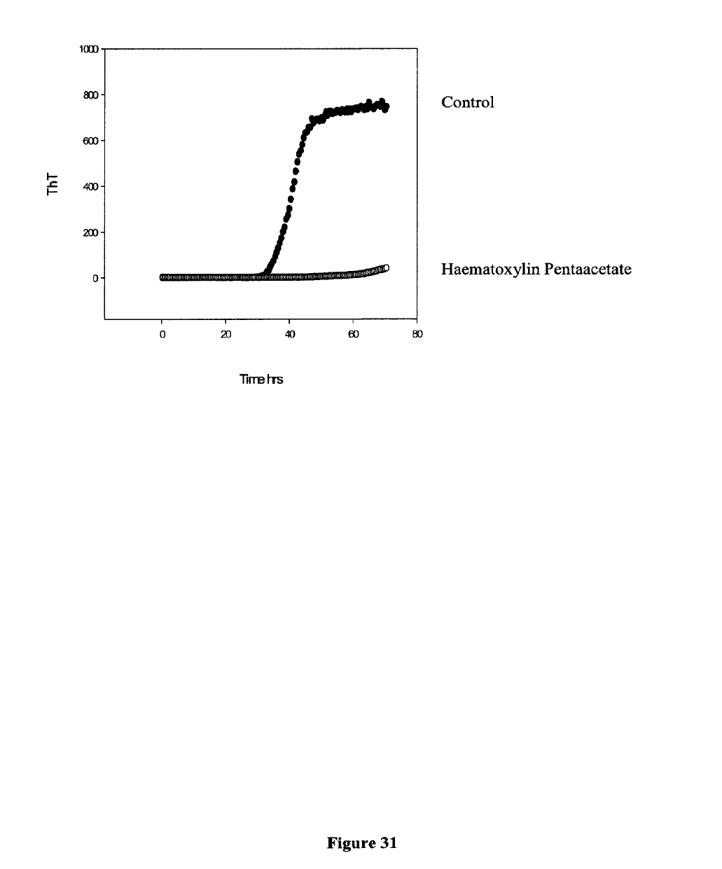

FIG. 31 shows Thioflavin T assay results from experiments with haematoxylin pentaacetate.

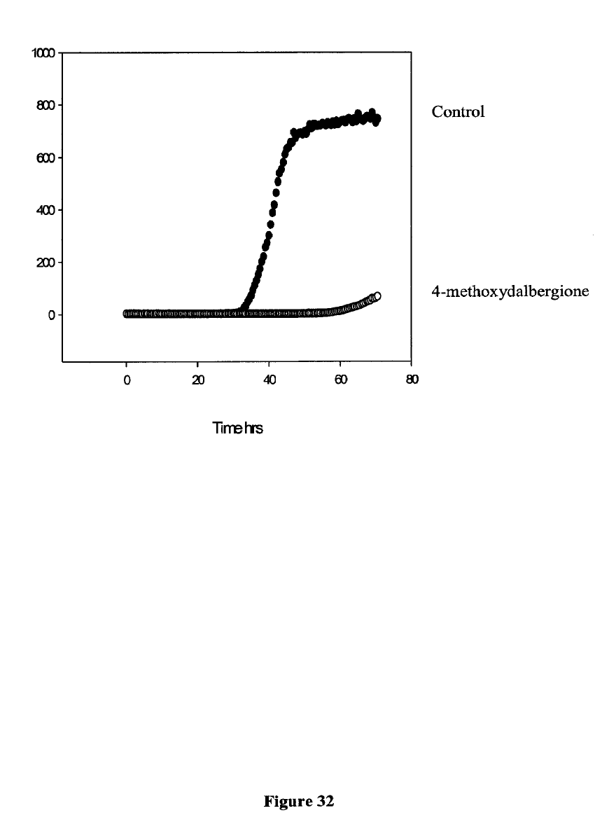

FIG. 32 shows Thioflavin T assay results from experiments with 4-methoxydalbergione.

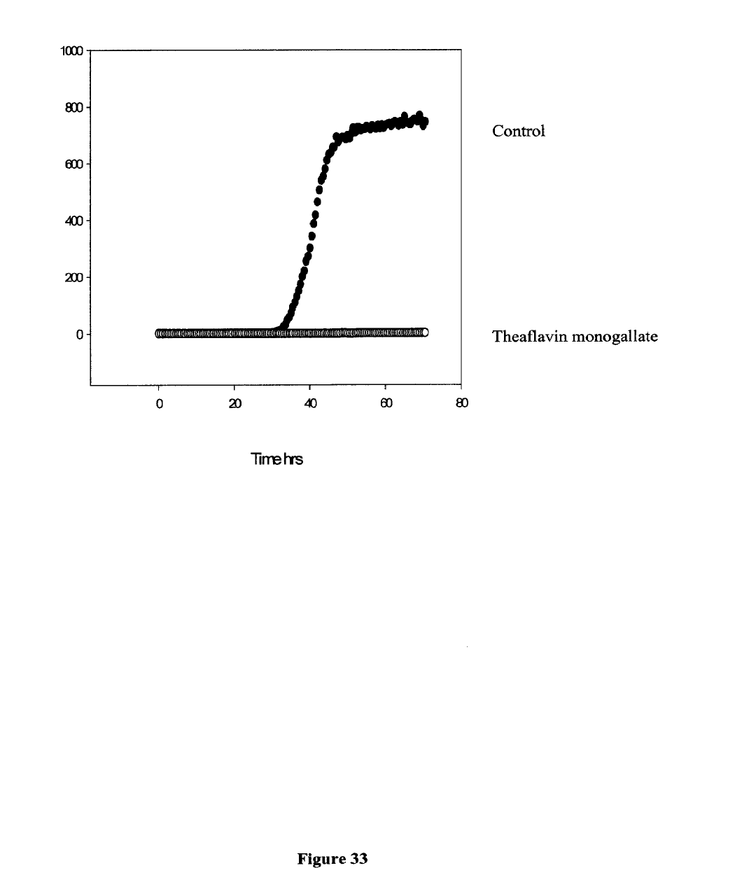

FIG. 33 shows Thioflavin T assay results from experiments with theaflavin monogallate.

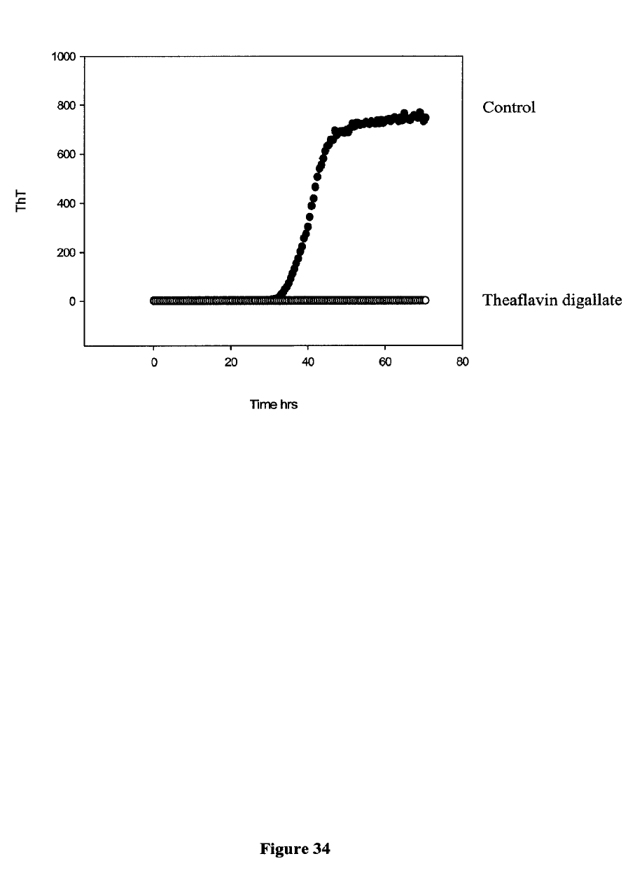

FIG. 34 shows Thioflavin T assay results from experiments with theaflavin digallate.

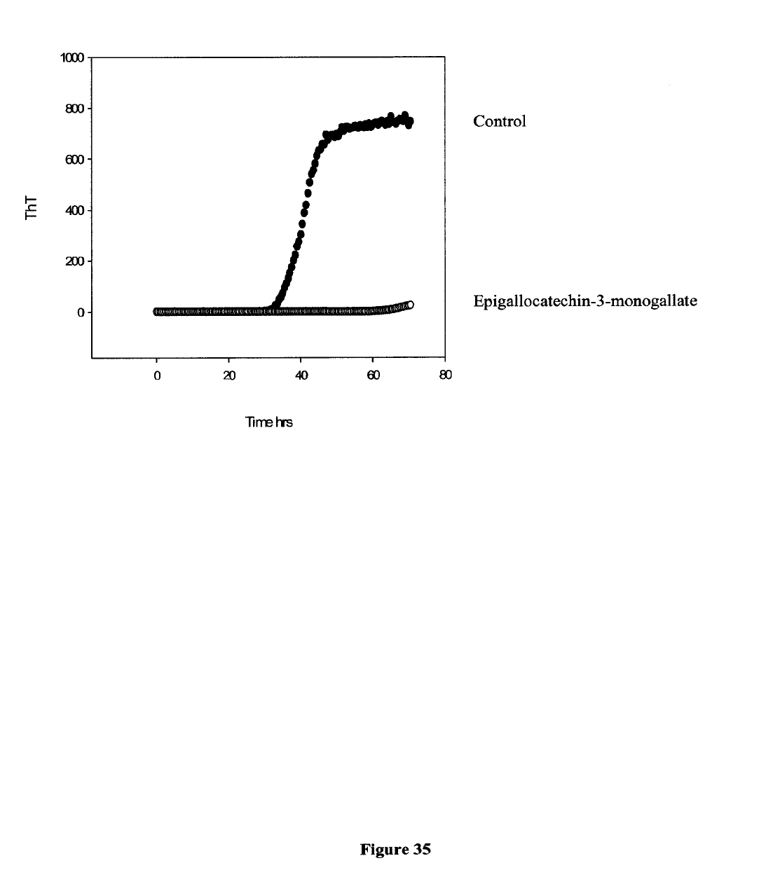

FIG. 35 shows Thioflavin T assay results from experiments with epigallocatechin-3-monogallate.

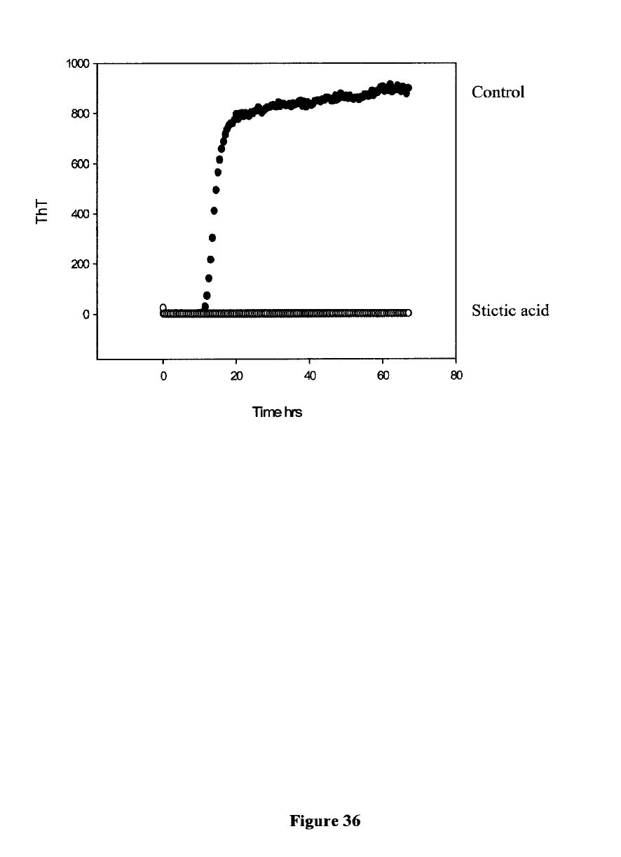

FIG. 36 shows Thioflavin T assay results from experiments with stictic acid

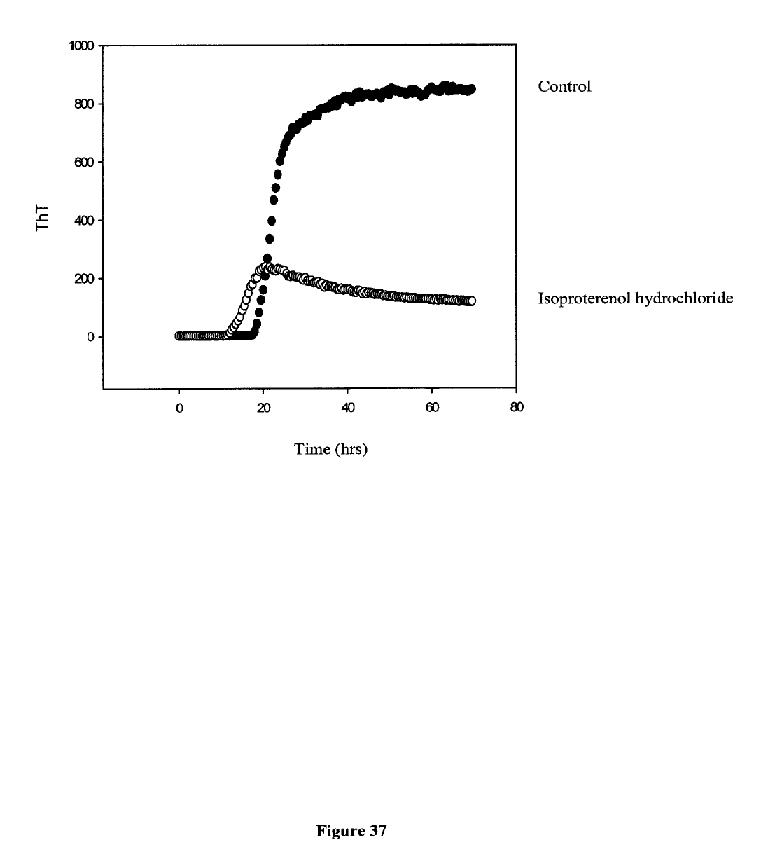

FIG. 37 shows Thioflavin T assay results from experiments with isoproterenol hydrochloride

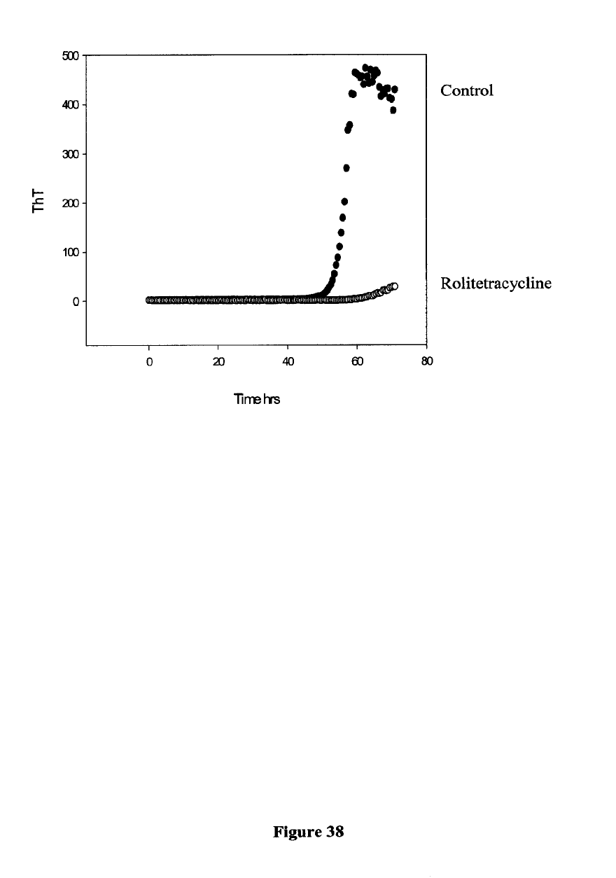

FIG. 38 shows Thioflavin T assay results from experiments with rolitetracycline.

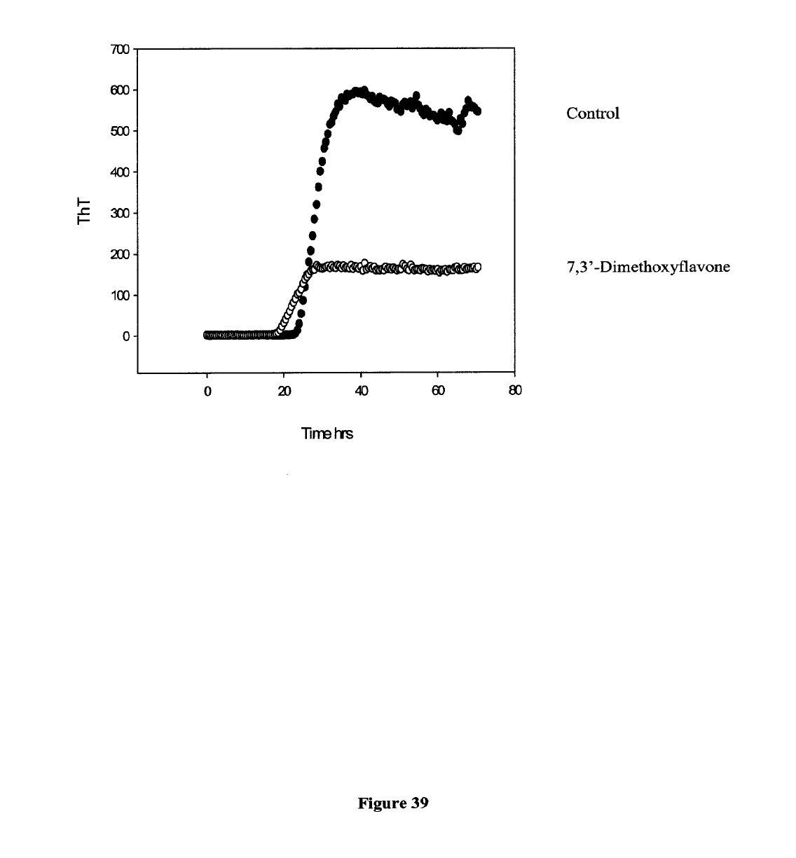

FIG. 39 shows Thioflavin T assay results from experiments with 7,3′-Dimethoxyflavone.

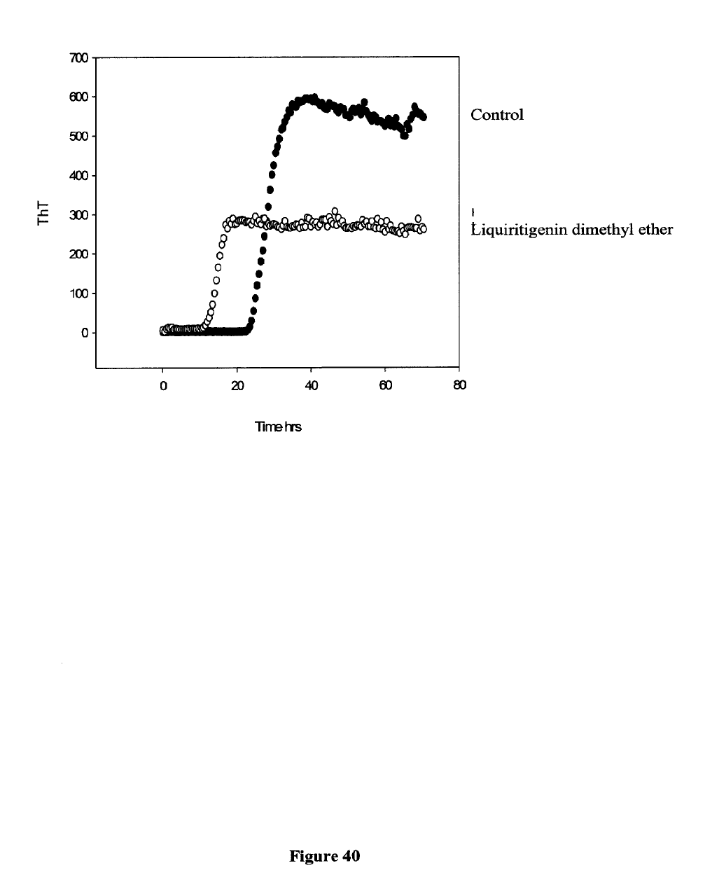

FIG. 40 shows Thioflavin T assay results from experiments with liquiritigenin dimethyl ether.

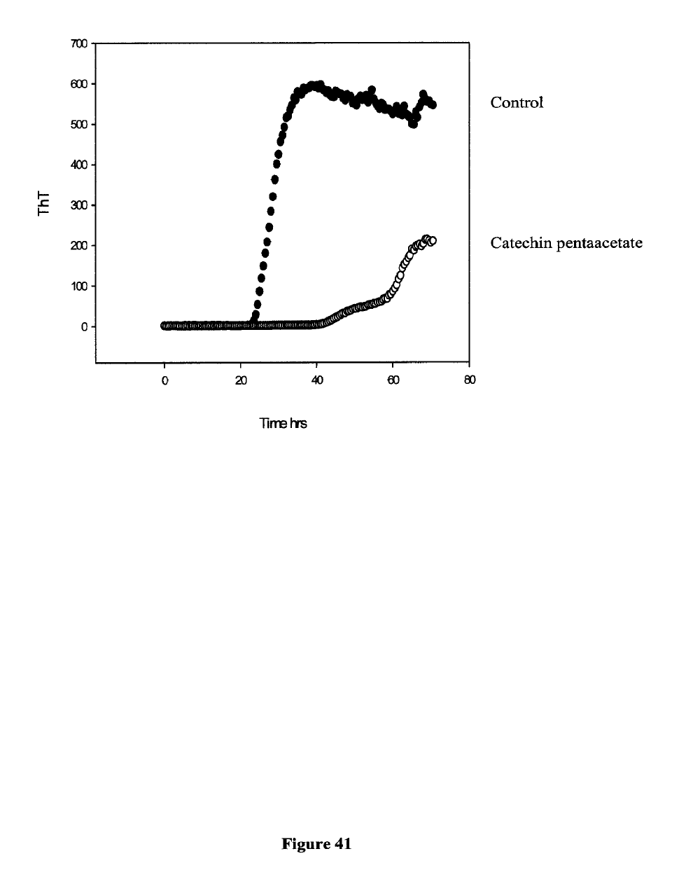

FIG. 41 shows Thioflavin T assay results from experiments with catechin pentaacetate

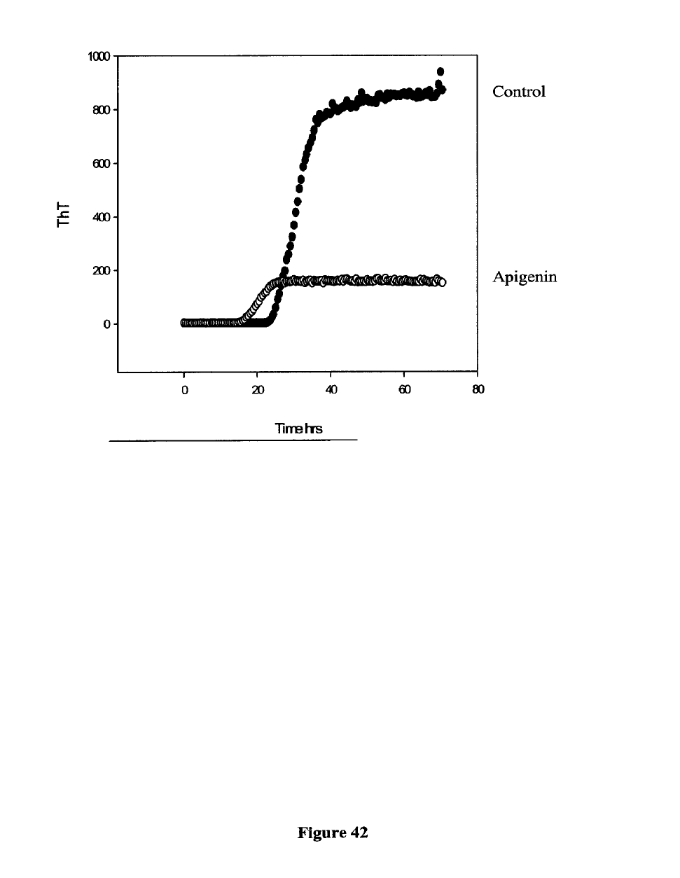

FIG. 42 shows Thioflavin T assay results from experiments with apigenin.

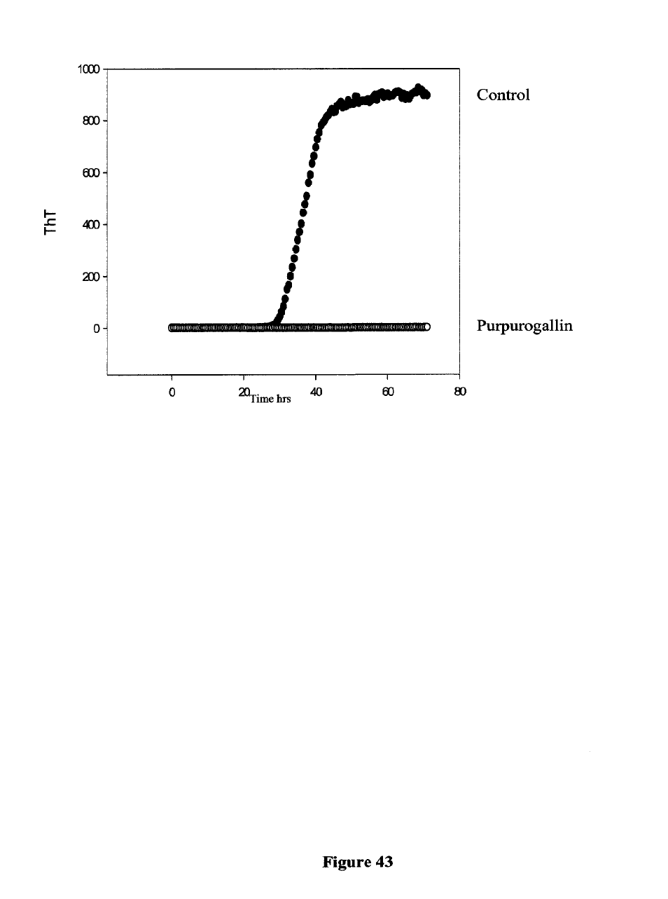

FIG. 43 shows Thioflavin T assay results from experiments with purpurogallin.

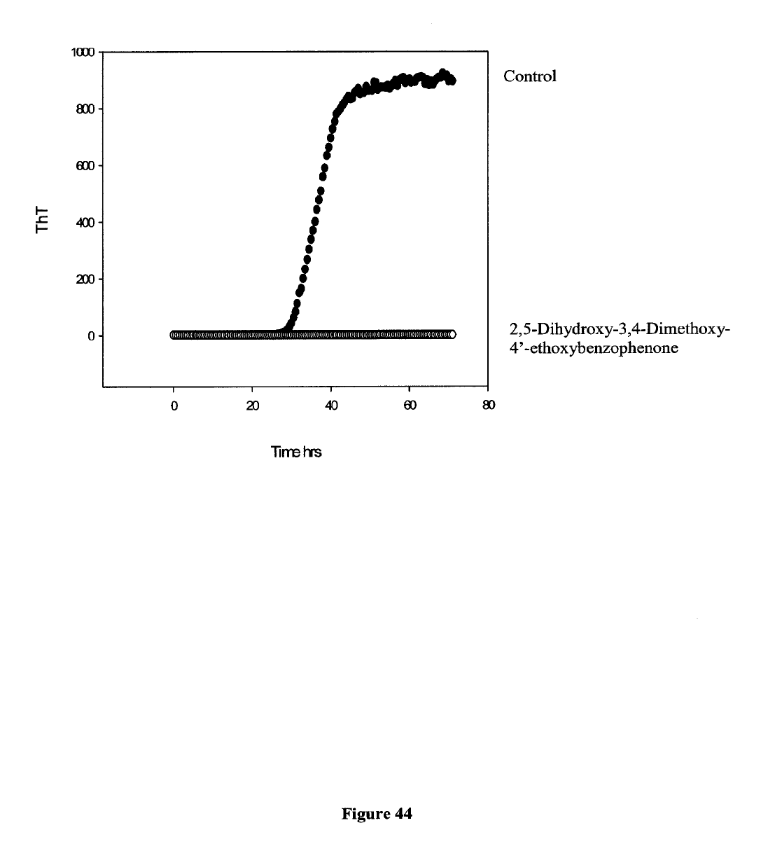

FIG. 44 shows Thioflavin T assay results from experiments with 2,5-dihydroxy-3,4-dimethoxy-4′-ethoxybenzophenone.

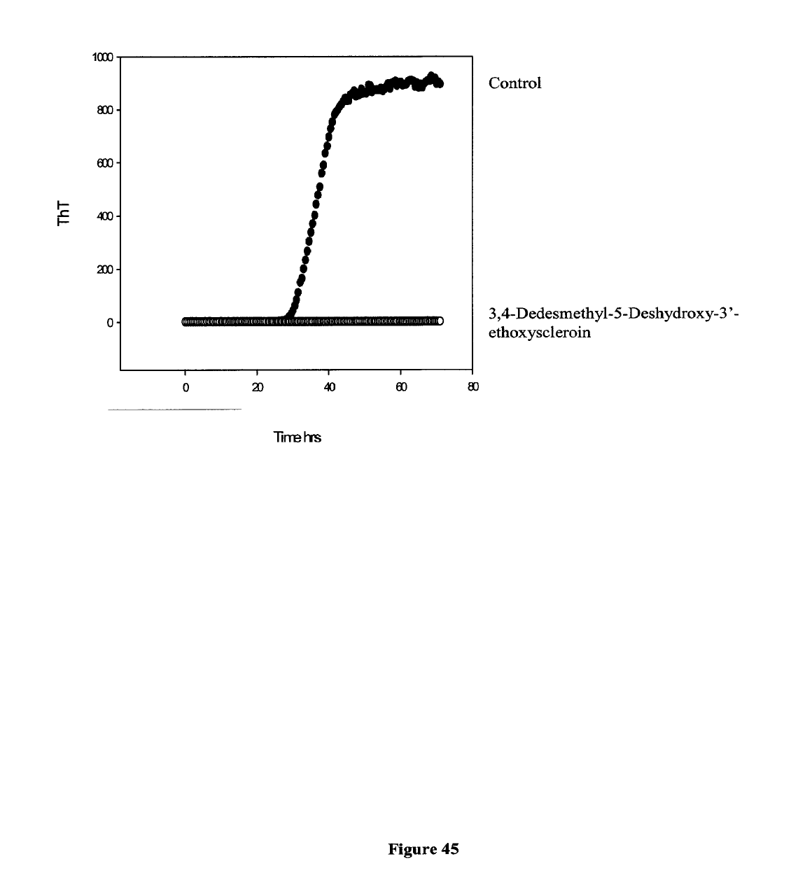

FIG. 45 shows Thioflavin T assay results from experiments with 3,4-dedesmethyl-5-deshydroxy-3′-ethoxyscleroin.

FIG. 46 shows results from experiments with atomic force microscopy for analysis of fibrillation.

### Example 5

### High Throughput Screening for Compounds that Inhibit, Decrease, Reverse, or Prevent α-Synuclein Fibrillation and/or Aggregation

A high throughput assay is achieved by using multi-well plates (96, 384, or higher density plates, nano plates) for test compound testing. Each well in each plate can be used as a miniature test-tube and mutltiple reactions can be studied at a single time. Lead compounds are then tested for potential therapeutic effectiveness in increasingly complex and relevant biological systems. Without being limited to any theory, if these compounds can be developed for therapeutic use, and administered at the earliest sign of disease, or even prophylactically for subjects. It could come close to achieving or in fact achieve a cure.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A prophylactic method for a neurological disorder comprising, administering a compound selected from Table 5 to a subject at risk of developing said disorder.

2. The method of claim 1 wherein said disorder is selected from a group consisting of Parkinson's disease, Alzheimer's disease, Huntington's disease, multiple system atrophy or Lewy body diseases.

3. The method of claim 1 wherein said disorder is Parkinson's disease.

4. A method of treating a subject at risk of developing a neurological disorder comprising, administering one or more compounds selected from Table 5 to said subject, wherein said subject does not show any primary symptoms associated with said disorder; and wherein said administering delays or prevents onset of primary symptoms of said disorder.

5. The method of claim 4 wherein said disorder is selected from a group consisting of Parkinson's disease, Alzheimer's disease, Huntington's disease, multiple system atrophy or Lewy body disease.

6. The method of claim 4 wherein said disorder is Parkinson's disease.

7. The method of claim 4 wherein said subject displays at least two secondary symptoms associated with said disorder prior to administering.

8. The method of claim 4 wherein said subject displays at least one secondary symptom and carries at least one genetic marker associated with said disorder prior to administering.

9. The method of claim 4 wherein said subject displays at least one secondary symptom and has at least one biomarker associated with said disorder prior to administering.

10. The method of claim 4 wherein said subject displays no secondary symptoms.

11. A method of preventing or reversing α-synuclein fibrillation in a subject with or at risk of developing a neurological disorder comprising administering an effective amount of a compound that prevents or reverses α-synuclein fibrillation, wherein said compound is selected from the group listed in Table 5.

12. The method of claim 11 wherein said disorder is selected from a group consisting of Parkinson's disease, Alzheimer's disease, Huntington's disease, multiple system atrophy or Lewy body disease.

13. The method of claim 11 wherein said disorder is Parkinson's disease.

14. The method of any of claim 1, 4 or 11 wherein said subject is at risk of developing the neurological disorder and risk is determined by genetic testing of a sample from said subject.

15. The method of any of claim 1, 4 or 11 wherein said subject is at risk of developing the neurological disorder and said risk is determined by testing for biomarkers from a sample from said subject.

16. The method of any of claim 1, 4 or 11 wherein said subject is at risk of developing the neurological disorder and said risk is determined by screening for secondary symptoms.

17. The method of claim 16 wherein said disease is Parkinson's disease and said secondary symptom is selected from the group consisting of rapid eye movement sleep behavioral disorder, olfactory dysfunction, cardiac sympathetic denervation, constipation, depression, anxiety and dementia.

18. The method of any of claim 1, 4 or 11 wherein said compound is apomorphine, epigallocatechin gallate, baicalein, quercetin, or curcumin.

19. The method of any of claim 1, 4 or 11 wherein said administering is via an oral route, continuous duodenal infusion, rectal, intranasal, sublingual and subcutaneous, skin patches, prodrug-dispensing liposomes, pulmonary delivery or a combination thereof.

20. The method of any of claim 1, 4 or 11 further comprising delivering an additional therapeutic agent to achieve a therapeutic effect in combination with said compound.

21. The method of claim 20 wherein said additional therapeutic agent is selected from a group consisting of an anti-emetic, 1-dihydroxyphenylalanine, aromatic acid decarboxylase inhibitor, catechol-O-methyltransferase inhibitor, monoamine oxidase inhibitor, tocopherol, a different dopamine agonist, otigotine, lisuride, nicotinic receptor agonist, amantadine, carbidopa, entacapone, levodopa, bromocriptine, pergolide, pramixpexole, cabergoline, ropinorole, or a combination of two or more thereof.

22. The method of claim 20 wherein said therapeutic effect comprises reducing said risk of developing said disorder.

23. The method of claim 20 wherein said delivering is before, concurrent or after said administering.

24. The method of claim 20 wherein said therapeutic agent is levodopa, a nicotine receptor modulator, or a monoamine oxidase inhibitor.

25. A method of treating Parkinson's disease in a subject comprising administering to the subject an effective amount of any compound of Table 5 except apomorphine, levodopa, or benzerazide.

26. The method of claim 25 wherein said compound is epigallocatechin gallate, baicalein, quercetin, or curcumin.

27. The method of claim 25 wherein said administering is via an oral route, continuous duodenal infusion, rectal, intranasal, sublingual and subcutaneous, skin patches, prodrug-dispensing liposomes, pulmonary delivery or a combination thereof.

28. The method of claim 25 further comprising delivering an additional therapeutic agent to achieve a therapeutic effect in combination with said compound.

29. The method of claim 28 wherein said additional therapeutic agent is selected from a group consisting of an anti-emetic, 1-dihydroxyphenylalanine, aromatic acid decarboxylase inhibitor, catechol-O-methyltransferase inhibitor, monoamine oxidase inhibitor, tocopherol, a different dopamine agonist, otigotine, lisuride, nicotinic receptor agonist, amantadine, carbidopa, entacapone, levodopa, bromocriptine, pergolide, pramixpexole, cabergoline, ropinorole, or a combination of two or more thereof.

30. A method of treating a subject at risk of developing a neurological disorder comprising, administering either a dopamine agonist, a monoamine oxidase inhibitor, a nutraceutical, or an inhibitor of α-synuclein fibrillation or any combination thereof to said subject, wherein said subject does not show any primary symptoms associated with said disorder; and wherein said administering delays or prevents onset of primary symptoms of said disorder.

31. The method of claim 30 wherein the neurological disorder is Parkinson's disease.

32. The method of claim 31 wherein the risk of developing Parkinson's disease is determined by one or more of the following methods: screening for a genetic trait, screening for secondary symptoms, or screening for biomarkers.

33. The method of claim 32 wherein the genetic trait is a mutation in a gene.

34. The method of claim 33 wherein said mutation is selected from the group consisting of α-synuclein substitution, deletion, insertion, triplication and duplication.

35. The method of claim 32 wherein said genetic trait is a polymorphism.

36. The method of claim 35 wherein said polymorphism is selected from the group consisting of SNP, STR and VNTR.

37. The method of claim 30, wherein said subject is from about 20 to about 40 years old, about 30 to 50, or about 40 to 70 years old.

38. The method of claim 30, wherein said administering is via an oral route, continuous duodenal infusion, rectal, intranasal, sublingual and subcutaneous, skin patches, prodrug-dispensing liposomes, pulmonary delivery or a combination thereof.

39. The method of claim 30, further comprising delivering an additional therapeutic agent to achieve a therapeutic effect in combination with said compound.

40. The method of claim 30, wherein said therapeutic agent is selected from a group consisting of an anti-emetic, 1-dihydroxyphenylalanine, aromatic acid decarboxylase inhibitor, catechol-O-methyltransferase inhibitor, monoamine oxidase-B inhibitor, a different dopamine agonist, otigotine, lisuride, nicotinic receptor agonist, amantadine, carbidopa, entacapone, levodopa, a tocopherol, bromocriptine, pergolide, pramixpexole, cabergoline, ropinorole, or a combination of two or more thereof.

41. The method of claim 39, wherein said therapeutic effect comprises enhancing said delayed progression and/or decreased side effects associated with said administering.

42. The method of claim 39, wherein said delivering is before, concurrent or after said administering.

43. The method of claim 39, wherein said therapeutic agent is levodopa, a nicotinic receptor modulator, or a monoamine oxidase inhibitor.

44. A method of treating a subject at risk of or suffering from a neurological disorder comprising, administering one or more compounds selected from Table 5 to said subject, wherein said administering decreases, delays, prevents, or reverses primary symptoms of said disorder.

45. The method of claim 44 wherein said compound is epigallocatechin gallate, baicalein, quercetin, or curcumin.

46. The method of claim 44 wherein said administering is via an oral route, continuous duodenal infusion, rectal, intranasal, sublingual and subcutaneous, skin patches, prodrug-dispensing liposomes, pulmonary delivery or a combination thereof.

47. The method of claim 44 further comprising delivering an additional therapeutic agent to achieve a therapeutic effect in combination with said compound.

48. The method of claim 44 wherein said additional therapeutic agent is selected from a group consisting of an anti-emetic, 1-dihydroxyphenylalanine, aromatic acid decarboxylase inhibitor, catechol-O-methyltransferase inhibitor, monoamine oxidase inhibitor, tocopherol, a different dopamine agonist, otigotine, lisuride, nicotinic receptor agonist, amantadine, carbidopa, entacapone, levodopa, bromocriptine, pergolide, pramixpexole, cabergoline, ropinorole, or a combination of two or more thereof
